(19) United States
(12) Patent Application Publication
(10) Pub. No.: US 2026/0193283 A1
(43) Pub. Date: Jul. 9, 2026

(54) CARBOXAMIDE DERIVATIVE HAVING RSK INHIBITORY EFFECT, PHARMACEUTICAL COMPOSITION COMPRISING SAME, AND USE THEREOF

(71) Applicant: KINOTECK THERAPEUTICS CO., LTD., Guangzhou Guangdong (CN)

(72) Inventors: Shaohua CHANG, Guangzhou Guangdong (CN); Xiaofei CHEN, Guangzhou Guangdong (CN); Ning YAN, Guangzhou Guangdong (CN); Ping LI, Guangzhou Guangdong (CN); Dawei FANG, Guangzhou Guangdong (CN); Xiaomei REN, Guangzhou Guangdong (CN); Dawei MA, Guangzhou Guangdong (CN)

(21) Appl. No.: 18/872,452

(22) PCT Filed: Jun. 6, 2023
(86) PCT No.: PCT/CN2023/098656

§ 371 Date: Aug. 26, 2025

(30) Foreign Application Priority Data
Jun. 7, 2022 (CN) 202210638658.X

(51) Int. Cl. C07F 9/6561; A61K 31/4985; A61K 31/675; A61P 35/00; C07B 59/00; C07D 471/04; C07D 471/14

(52) U.S. Cl. CPC C07F 9/6561; A61K 31/4985; A61K 31/675; A61P 35/00; C07B 59/002; C07D 471/04; C07D 471/14

(57) ABSTRACT

Provided in the present invention are a carboxamide derivative having an RSK inhibitory effect, a pharmaceutical composition comprising same, and the use thereof. Specifically, provided in the present invention are a carboxamide derivative as represented by formula (I), which can be used as a p90 ribosomal S6 kinase (RSK) inhibitor, a tautomer, a stereoisomer, a hydrate, a solvate and a pharmaceutically acceptable salt thereof, a pharmaceutical composition comprising the compound, and the use thereof in the preparation of a pharmaceutical composition.

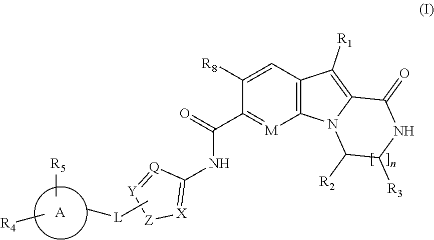

### TECHNICAL FIELD

The present invention relates to the field of medicinal chemistry, specifically, the present invention provides a structural novel RSK inhibitor.

### BACKGROUND

The Ras-MAPK pathway and PI3K-PDK1 pathway are usually abnormally active in many cancers, and the cascaded MAPK and PI3K signaling levels converge on the activation of downstream protein kinases RSK and YB-1, leading to poor prognosis and resistance to chemotherapy and radiotherapy. The p90 ribosomal S6 kinase (RSKs) family belongs to serine/threonine kinases, which are composed of RSK1, RSK2, RSK3, and RSK4. RSK1-4 has high sequence homology (73-80%), but the difference between N-terminus and C-terminus is greatest. RSK consists of two kinase domains with different functions (an N-terminal kinase domain (NTKD) and a C-terminal kinase domain (CTKD)) connected by a regulatory linker domain. The activation of RSK requires continuous phosphorylation of ERK1/2 in CTKD, self phosphorylation of the linker domain, and regulation of various cellular processes such as cell growth, proliferation, survival, and motility through phosphorylation of its substrates such as YB-1, GSK30, BAD, procaspase-8, TSC2, and c-Fos.

The expression status of RSK1-3 is widely present, and RSK1 and RSK2 are the most common isoforms in cancer, their expression and activation promote tumor growth and survival, and RSK3 is usually not expressed in cancer, however, like RSK1 and RSK2, it is associated with drug resistance. RSK4 is mainly expressed during embryonic development, and its role in malignant tumors is not yet clear, maybe tissue-specific, RSK4 mainly exhibits cytoplasmic characteristics and constitutive activity status, as well as kinase activity status independent of growth elements. A variety of tumors related to RSK, including but not limited to breast cancer, prostate cancer, lung cancer, brain cancer, blood cancer, skin cancer, bone cancer and ovarian cancer, are treated by inhibiting the activation of RSK, and by eliminating cancer stem cells (CSC) or tumor initiating cells (TIC) and other mechanisms. It has been reported that about 70% of locally advanced breast cancer patients have RSK activation, mainly ER+/PR+, and about 85% of TNBC tumors have RSK activation, and the research shows that RSK inhibitors can cause tumor cell apoptosis and inhibit tumor metastasis, and they will not cause Akt activation compared with MEK inhibitors, they have more advantages in drug combination to solve drug resistance, providing a new treatment approach for treating TNBC. In the field of prostate cancer treatment, the combination of RSK2 and p300 can excitedly regulate the transcription program of AR, and RSK inhibitors can block hormone pathways and the YB-1 pathway-mediated drug resistance, and there are good prospects for RSK inhibitors in the treatment of castration resistant prostate and combination therapy. In the field of acute myeloid leukemia (AML) treatment, the high expression and abnormal activation of RSK subtypes lead to adverse outcomes and chemotherapy resistance, RSK inhibitors can block the Ser133 phosphorylation of CREB, thereby blocking the promotion of proliferation and survival of myeloid cells by inducing the expression of Bcl-2, cyclin A, and cyclin D.

Previous studies have confirmed that RSK plays an important role in various life activities such as cell growth, proliferation, apoptosis, and transformation, and increased activation of RSK is involved in multiple pathological etiologies, including various types of cancer, cardiovascular disease, liver and lung fibrosis, and infections. In recent years, many RSK small molecule inhibitors have emerged, such as SLO101, BI-D1870, FMK, as well as PMD-026, some RSK inhibitors (PMD-026) have entered the clinical stage, and some experiments have achieved encouraging early results (NCT04115306), but the pharmacokinetic properties of most molecules are poor, which is not conducive to in vivo research. Therefore, the development of RSK inhibitors with better activity and pharmacokinetics has promising application prospects in the pharmaceutical industry.

### SUMMARY OF THE INVENTION

The purpose of the present invention is to provide a carboxamide derivative having RSK inhibitory effect.

The first aspect of the present invention provides a compound represented by formula (I), a tautomer, a stereoisomer, a hydrate, a solvate, or a pharmaceutically acceptable salt thereof:

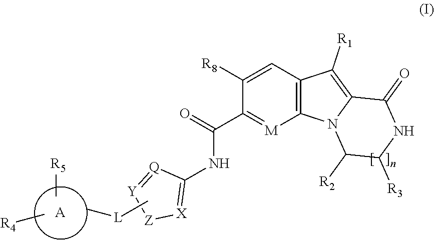

wherein,
n is 1 or 2;
M is independently N or CRₐ;
Rₐ is selected from the group consisting of: halogen, cyano, and C₁₋₄ alkoxy;
X, Y and Q are each independently selected from the group consisting of: CR_{b}, or N; Z is selected from the group consisting of: S, O, or NR_{b}; and

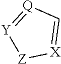

is an aromatic ring, when the connecting site is located at CR_{b} or NR_{b}, the CR_{b} or NR_{b} is C or N;
L is selected from the group consisting of: chemical bond, —C(═O)—, CHR_{b}, C(R_{b})₂, NH or O;
ring A is selected from the group consisting of: C₆₋₁₀ aryl, and 5-12 membered heteroaryl;
R₁ is selected from the group consisting of: H, D, halogen, amino, and C₁₋₄ alkyl;
R₂ and R₃ are each independently selected from the group consisting of: H, D, C₁₋₆ alkyl, and C₁₋₆ haloalkyl, and R₂ and R₃ can be the same or different;
R₄ is selected from the group consisting of: H, D, halogen, hydroxyl, amino, C₂₋₆ sulfonyl, sulfinyl, —N═S(O)(CH₃)₂,

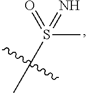 [SMILES: *S(C)(=N)=O]

phosphoryl(-P(O)(CH₃)₂), C₁₋₆ alkyl, C₁₋₆ alkoxy, C₁₋₆ haloalkyl, C₁₋₆ haloalkoxy, C₂₋₆ alkenyl, C₁₋₆ haloalkenyl, C₂₋₆ alkynyl, C₁₋₆ haloalkynyl, C₃₋₆ cycloalkyl, C₆₋₁₀ aryl, 5-12 membered heterocyclyl, and 5-12 membered heteroaryl; and the heterocyclyl and heteroaryl independently contain 1-3 heteroatoms selected from N, O, and S;
R₅ is selected from the group consisting of: H, halogen, hydroxyl, amino, sulfonyl, sulfinyl, phosphoryl, C₁₋₆ alkyl, C₁₋₆ alkoxy, C₁₋₆ haloalkyl, C₁₋₆ haloalkoxy, C₂₋₆ alkenyl, C₁₋₆ haloalkenyl, C₂₋₆ alkynyl, C₁₋₆ haloalkynyl, C₃₋₆ cycloalkyl, C₆₋₁₀ aryl, 5-12 membered heterocyclyl, and 5-12 membered heteroaryl; and the heterocyclyl and heteroaryl independently contain 1-3 heteroatoms selected from N, O, and S;
R₈ is selected from the group consisting of: H, and halogen;
and, R₁, R₂, R₃, R₄ and R₅ can be optionally substituted with one or more R_{b};
R_{b} is selected from the group consisting of: D, halogen, hydroxyl, cyano, amino, imino, C₁₋₄ alkyl, C₁₋₄ alkoxy, C₁₋₄ haloalkyl, C₁₋₄ haloalkoxy, C₂₋₄ alkenyl, C₁₋₄ haloalkenyl, C₂₋₄ alkynyl, C₁₋₄ haloalkynyl, C₃₋₆ cycloalkyl, C₆₋₁₀ aryl, and 5-12 membered heteroaryl;
and when the compound has the structure shown in formula I-1 and M is N, ring A is not a phenyl that is unsubstituted or substituted with a substituent selected from the group consisting of: halogen, C₁₋₆ alkyl, C₁₋₆ haloalkyl-N(R⁷)₂, and —C₁₋₆ alkyl-N(R⁷)₂; wherein, R⁷ is H or C₁₋₆ alkyl;

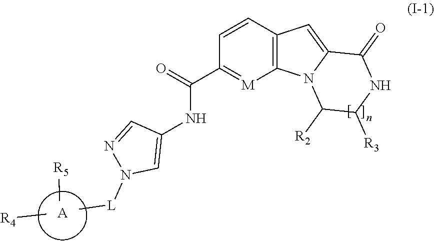

In some embodiments, the compound has the structure shown in formula I-1:

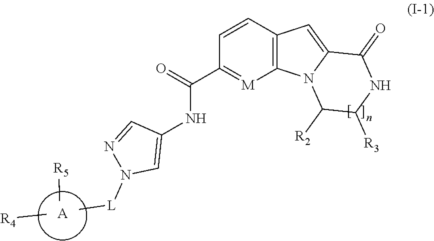

wherein, L is selected from the group consisting of: chemical bond, CH₂, CF₂, CHMe, and C(Me)₂;
when M is N, R₄ is H, D, halogen, C₁₋₆ alkyl, C₁₋₆ alkoxy, C₁₋₆ haloalkyl, or C₁₋₆ haloalkoxy; R₅ is —N═S(O)(CH₃)₂,

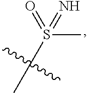 [SMILES: *S(C)(=N)=O]

phosphoryl(-P(O)(CH₃)₂), C₁₋₆ alkyl, C₃₋₆ cycloalkyl;

In some embodiments, the compound has a structure as shown in formula I-2:

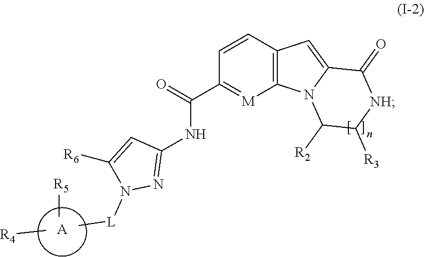

wherein,
L is selected from the group consisting of: chemical bond, CH₂, CF₂, CHMe, and C(Me)₂;
R₆ is selected from the group consisting of: H, D, halogen, C₁₋₆ alkyl, C₁₋₆ alkoxy, C₁₋₆ haloalkyl, C₁₋₆ haloalkoxy, C₂₋₆ alkenyl, C₁₋₆ haloalkenyl, C₂₋₆ alkynyl, C₁₋₆ haloalkynyl, C₃₋₆ cycloalkyl, C₆₋₁₀ aryl, 5-12 membered heterocyclyl, and 5-12 membered heteroaryl; and the heterocyclyl and heteroaryl independently contain 1-3 heteroatoms selected from N, O, and S;
and, R₆ can be optionally substituted with one or more R_{b}.

In some embodiments, the compound has a structure shown in formula I-3:

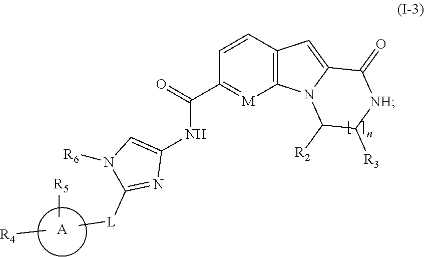

wherein, R₆ is selected from the group consisting of: H, C₁₋₆ alkyl, C₁₋₆ haloalkyl, C₃₋₆ cycloalkyl, C₆₋₁₀ aryl, 5-12 membered heterocyclyl, and 5-12 membered heteroaryl; the heterocyclyl and heteroaryl independently contain 1-3 heteroatoms selected from N, O, and S; and, the R₆ can be optionally substituted by one or more R_{b}.

In some embodiments, R₂ and R₃ are each preferably selected from the group consisting of: H, and methyl, R₂, R₃ are the same or different.

In some embodiments, R₄ and R₅ are independently selected from the group consisting of: H, halogen, amino, sulfinyl, phosphoryl and C₁₋₄ alkyl, and R₄ and R₅ can be each independently substituted by one or more R_{b}.

In some embodiments, the compound is selected from the group consisting of:

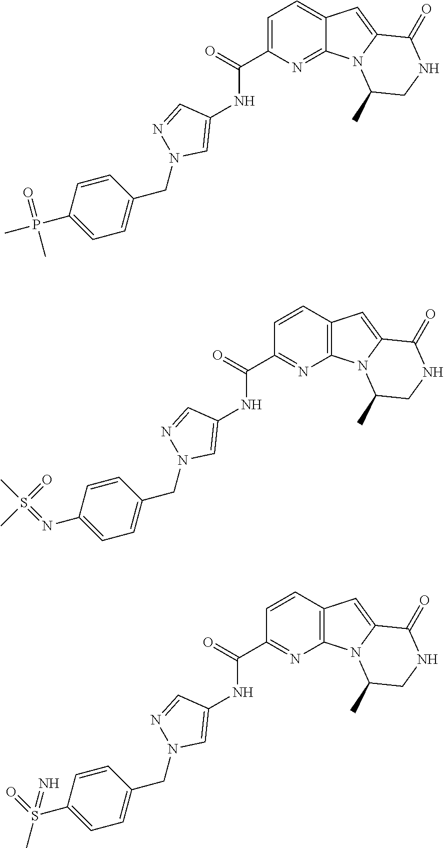 [SMILES: C[C@@H]1CNC(=O)c2cc3ccc(C(=O)Nc4cnn(Cc5ccc(P(C)(C)=O)cc5)c4)nc3n21 ; C[C@@H]1CNC(=O)c2cc3ccc(C(=O)Nc4cnn(Cc5ccc(N=S(C)(C)=O)cc5)c4)nc3n21 ; C[C@@H]1CNC(=O)c2cc3ccc(C(=O)Nc4cnn(Cc5ccc(S(C)(=N)=O)cc5)c4)nc3n21]

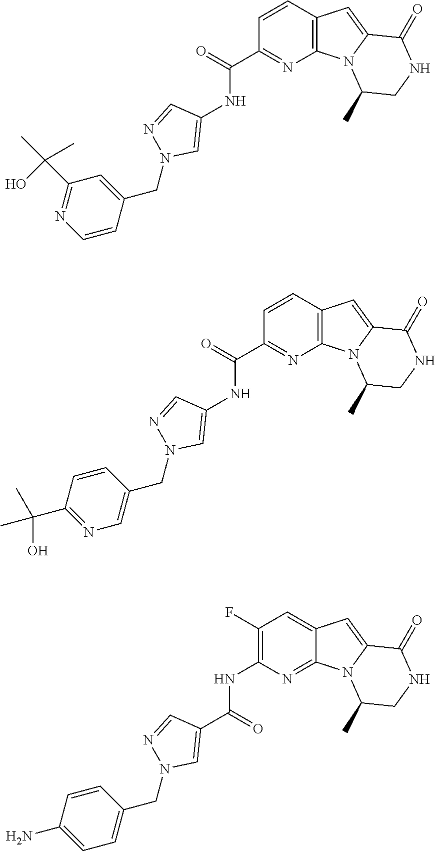 [SMILES: C[C@@H]1CNC(=O)c2cc3ccc(C(=O)Nc4cnn(Cc5ccnc(C(C)(C)O)c5)c4)nc3n21 ; C[C@@H]1CNC(=O)c2cc3ccc(C(=O)Nc4cnn(Cc5ccc(C(C)(C)O)nc5)c4)nc3n21 ; C[C@@H]1CNC(=O)c2cc3cc(F)c(NC(=O)c4cnn(Cc5ccc(N)cc5)c4)nc3n21]

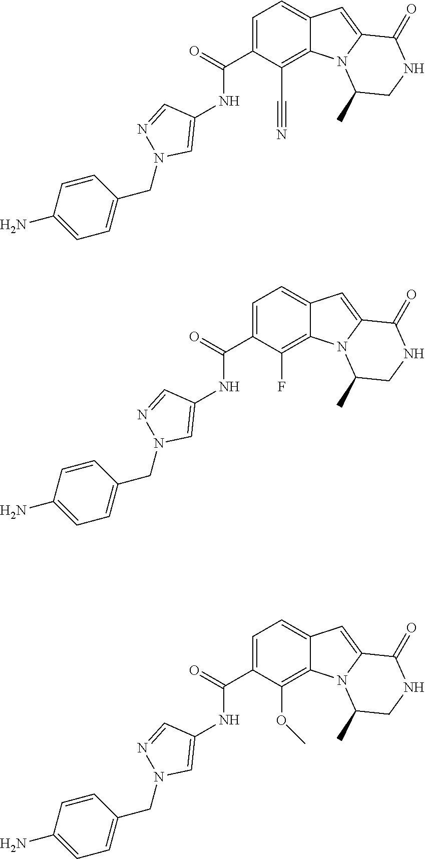 [SMILES: C[C@@H]1CNC(=O)c2cc3ccc(C(=O)Nc4cnn(Cc5ccc(N)cc5)c4)c(C#N)c3n21 ; C[C@@H]1CNC(=O)c2cc3ccc(C(=O)Nc4cnn(Cc5ccc(N)cc5)c4)c(F)c3n21 ; COc1c(C(=O)Nc2cnn(Cc3ccc(N)cc3)c2)ccc2cc3n(c12)[C@H](C)CNC3=O]

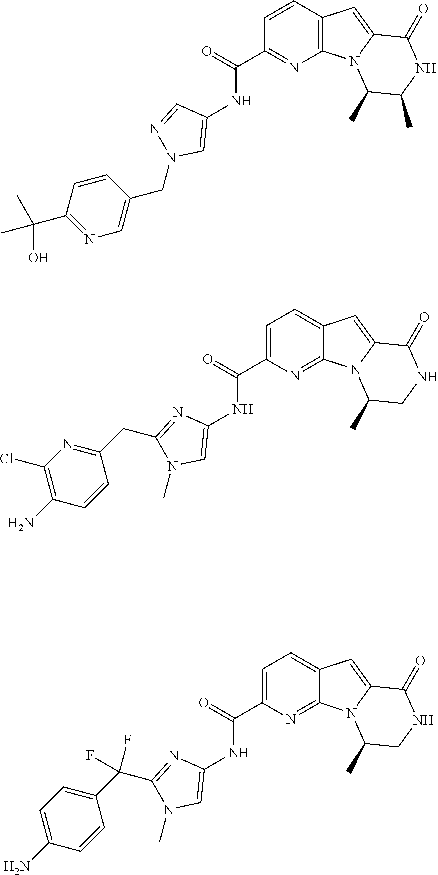 [SMILES: C[C@@H]1NC(=O)c2cc3ccc(C(=O)Nc4cnn(Cc5ccc(C(C)(C)O)nc5)c4)nc3n2[C@@H]1C ; C[C@@H]1CNC(=O)c2cc3ccc(C(=O)Nc4cn(C)c(Cc5ccc(N)c(Cl)n5)n4)nc3n21 ; C[C@@H]1CNC(=O)c2cc3ccc(C(=O)Nc4cn(C)c(C(F)(F)c5ccc(N)cc5)n4)nc3n21]

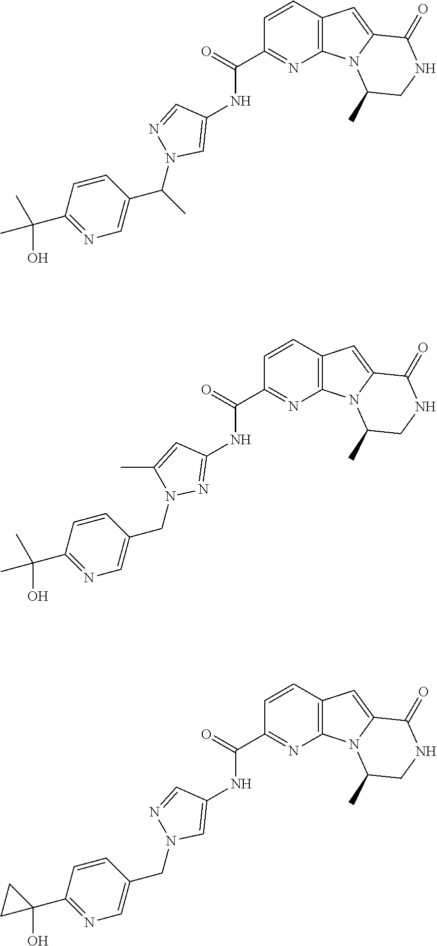 [SMILES: CC(c1ccc(C(C)(C)O)nc1)n1cc(NC(=O)c2ccc3cc4n(c3n2)[C@H](C)CNC4=O)cn1 ; Cc1cc(NC(=O)c2ccc3cc4n(c3n2)[C@H](C)CNC4=O)nn1Cc1ccc(C(C)(C)O)nc1 ; C[C@@H]1CNC(=O)c2cc3ccc(C(=O)Nc4cnn(Cc5ccc(C6(O)CC6)nc5)c4)nc3n21]

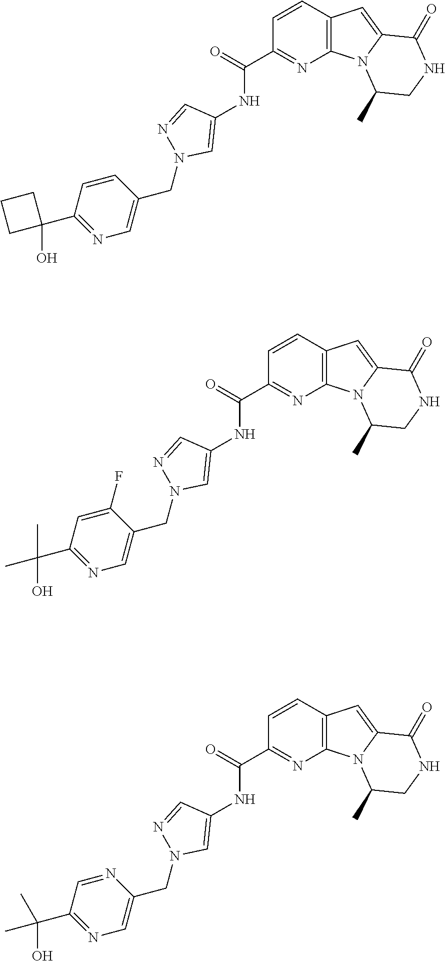 [SMILES: C[C@@H]1CNC(=O)c2cc3ccc(C(=O)Nc4cnn(Cc5ccc(C6(O)CCC6)nc5)c4)nc3n21 ; C[C@@H]1CNC(=O)c2cc3ccc(C(=O)Nc4cnn(Cc5cnc(C(C)(C)O)cc5F)c4)nc3n21 ; C[C@@H]1CNC(=O)c2cc3ccc(C(=O)Nc4cnn(Cc5cnc(C(C)(C)O)cn5)c4)nc3n21]

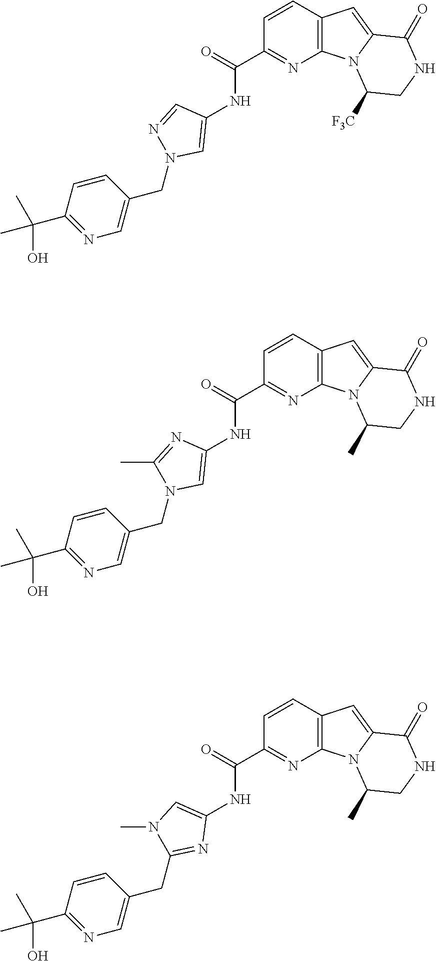 [SMILES: CC(C)(O)c1ccc(Cn2cc(NC(=O)c3ccc4cc5n(c4n3)[C@H](C(F)(F)F)CNC5=O)cn2)cn1 ; Cc1nc(NC(=O)c2ccc3cc4n(c3n2)[C@H](C)CNC4=O)cn1Cc1ccc(C(C)(C)O)nc1 ; C[C@@H]1CNC(=O)c2cc3ccc(C(=O)Nc4cn(C)c(Cc5ccc(C(C)(C)O)nc5)n4)nc3n21]

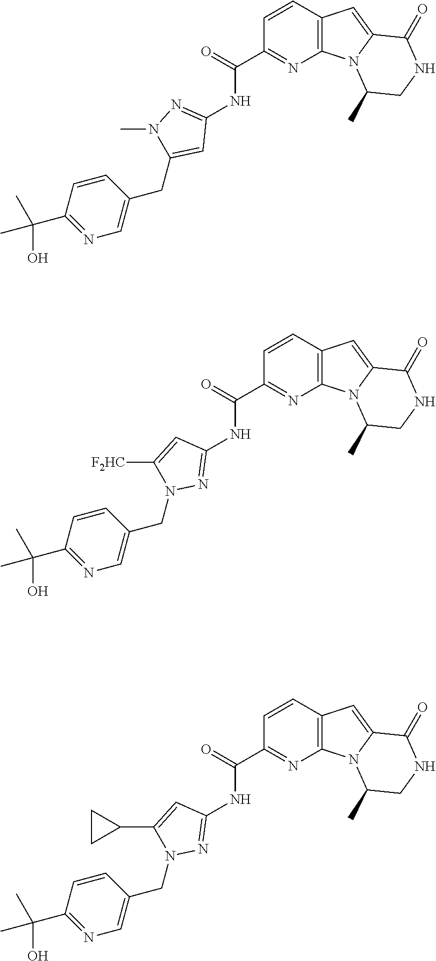 [SMILES: C[C@@H]1CNC(=O)c2cc3ccc(C(=O)Nc4cc(Cc5ccc(C(C)(C)O)nc5)n(C)n4)nc3n21 ; C[C@@H]1CNC(=O)c2cc3ccc(C(=O)Nc4cc(C(F)F)n(Cc5ccc(C(C)(C)O)nc5)n4)nc3n21 ; C[C@@H]1CNC(=O)c2cc3ccc(C(=O)Nc4cc(C5CC5)n(Cc5ccc(C(C)(C)O)nc5)n4)nc3n21]

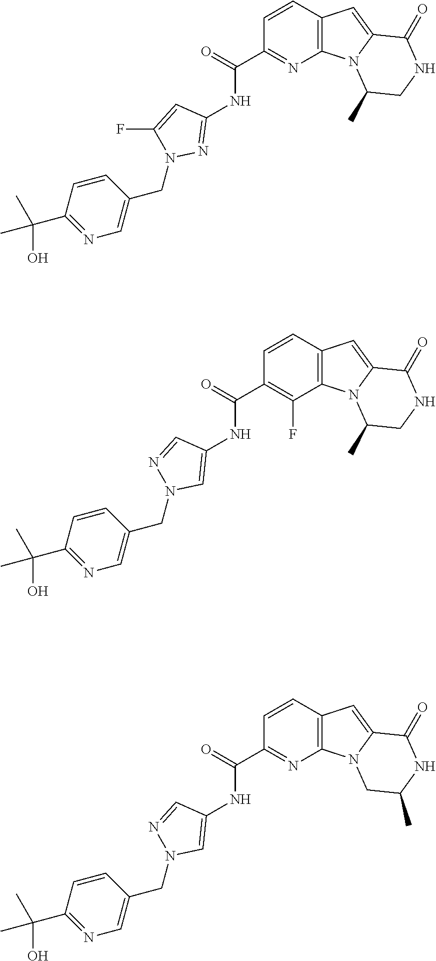 [SMILES: C[C@@H]1CNC(=O)c2cc3ccc(C(=O)Nc4cc(F)n(Cc5ccc(C(C)(C)O)nc5)n4)nc3n21 ; C[C@@H]1CNC(=O)c2cc3ccc(C(=O)Nc4cnn(Cc5ccc(C(C)(C)O)nc5)c4)c(F)c3n21 ; C[C@H]1Cn2c(cc3ccc(C(=O)Nc4cnn(Cc5ccc(C(C)(C)O)nc5)c4)nc32)C(=O)N1]

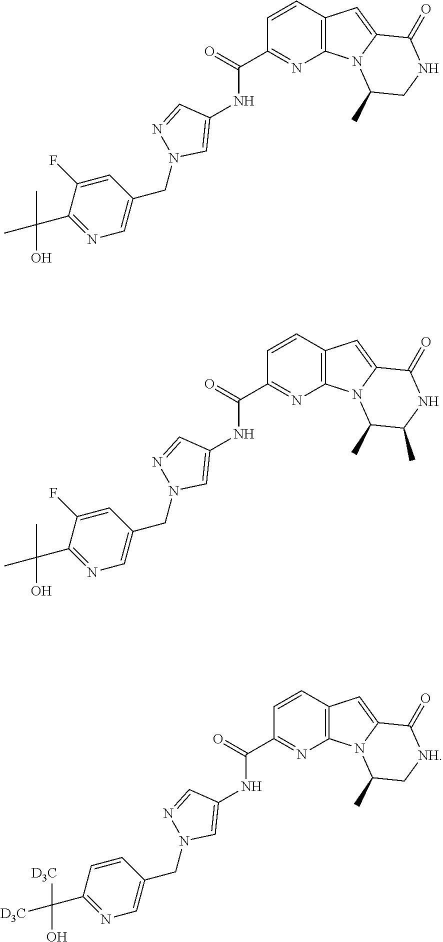 [SMILES: C[C@@H]1CNC(=O)c2cc3ccc(C(=O)Nc4cnn(Cc5cnc(C(C)(C)O)c(F)c5)c4)nc3n21 ; C[C@@H]1NC(=O)c2cc3ccc(C(=O)Nc4cnn(Cc5cnc(C(C)(C)O)c(F)c5)c4)nc3n2[C@@H]1C ; [2H]C([2H])([2H])C(O)(c1ccc(Cn2cc(NC(=O)c3ccc4cc5n(c4n3)[C@H](C)CNC5=O)cn2)cn1)C([2H])([2H])[2H]]

In the second aspect of the present invention, provided is a pharmaceutical composition comprising: one or more of a compound of formula I according to the first aspect of the present invention, a pharmaceutically acceptable salt, a racemate, an R-isomer, an S-isomer, or a mixture thereof, and one or more pharmaceutically acceptable carriers, excipients, adjuvants, excipients, and/or diluents.

In the third aspect of the present invention, provided is a use of the compound of formula I according to the first aspect of the present invention, a pharmaceutically acceptable salt, a racemate, an R-isomer, an S-isomer, or a mixture thereof, in the preparation of a pharmaceutical composition for diseases or conditions associated with p90 ribosomal S6 kinase (RSK) activity.

In another preferred embodiments, the disease or condition is cancer.

In some embodiments, the disease or condition is selected from the group consisting of: breast cancer, prostate cancer, lung cancer, brain cancer, skin cancer, bone cancer, ovarian cancer, multiple myeloma or leukemia.

It should be understood that within the scope of the present invention, the above-mentioned technical features of the present invention and the technical features specifically described in the following (such as examples) can be combined with each other to form a new or preferred technical solution. Limited to space, it will not be elaborated here.

### DETAILED DESCRIPTION OF THE EMBODIMENT

After long-term and in-depth research, the inventor has provided a carboxamide derivative having RSK inhibitory activity, the derivative has good RSK inhibitory activity and can be used for preventing, treating, and/or alleviating indications related to RSK activation. The inventor has completed the present invention based on the above findings.

### Definition

As used herein, the term “alkyl” includes straight or branched alkyl. For example, a C₁-C₈ alkyl refers to a straight or branched alkyl with 1-8 carbon atoms, such as methyl, ethyl, propyl, isopropyl, butyl, isobutyl, tert-butyl, etc.

As used herein, the term “alkenyl” includes straight or branched alkenyl. For example, a C₂-C₆ alkenyl refers to a straight or branched alkenyl with 2-6 carbon atoms, such as vinyl, allyl, 1-propenyl, isopropenyl, 1-butenyl, 2-butenyl, or similar groups.

As used herein, the term “alkynyl” includes straight or branched alkynyl. For example, C₂-C₆ alkynyl refers to a straight or branched alkynyl with 2-6 carbon atoms, such as ethynyl, propynyl, butynyl, or similar groups.

As used herein, the term “cycloalkyl” refers to a cyclic saturated fatty hydrocarbon group with a specific number of carbon atoms. For example, a C₃-C₁₀ cycloalkyl refers to a cyclic saturated fatty hydrocarbon group with 3-10 carbon atoms. It can be a monocyclic ring, such as cyclopropyl, cyclobutyl, cyclopentyl, cyclohexyl, or similar groups. It can also be in the form of a bicyclic ring, such as a bridged ring or a spiro ring.

As used herein, the term “alkylamine” refers to an amine substituted with alkyl. For example, “C₁-C₈ alkylamino” refers to an amino substituted with C₁-C₈ alkyl, which can be mono- or di-substituted; for example, methylamine, ethylamine, propylamine, isopropylamine, butylamine, isobutyramimine, tert-butylamine, dimethylamine, diethylamine, dipropylamine, diisopropylamine, dibutylamine, diisobutylamine, di tert-butylamine, etc.

As used herein, the term “alkoxy” refers to a group having an alkyl-oxy structure. For example, “C₁-C₈ alkoxy” refers to a straight or branched alkoxy with 1-8 carbon atoms, including methoxy, ethoxy, propoxy, isopropoxy, butoxy, isobutoxy, tert-butoxy, etc.

As used herein, the term “haloalkyl” refers to an alkyl in which one or more hydrogen atoms are substituted with halogens, and the definition of alkyl is as described above.

As used herein, the term “haloalkoxy” refers to an alkoxy in which one or more hydrogen atoms are substituted with halogens, and the definition of alkoxy is as described above.

As used herein, the term “heterocyclyl” or “heterocyclic alkyl” refers to a saturated or partially saturated cyclic group with a specific number of ring atoms (such as 3-10 ring atoms), wherein 1-3 atoms are heteroatoms selected from N, S, and O. It can be in the form of a monocyclic ring, a bicyclic ring, or a polycyclic ring, such as a bridged ring or a spiro ring.

Specific examples can include oxetanyl, azetidinyl, tetrahydro-2H-pyranyl, piperidinyl, tetrahydrofuranyl, morpholinyl, and pyrrolidinyl.

As used herein, the term “C₆-C₁₀ aryl” refers to an aryl group with 6-10 carbon atoms, such as phenyl or naphthyl.

As used herein, the term “5-12 membered heteroaryl” refers to a cyclic aromatic group with 5-12 atoms, wherein 1-3 atoms are heteroatoms selected from the group consisting of N, S, and O. It can be in the form of a monocyclic ring or a fused ring. Specific examples can include pyridyl, pyridazinyl, pyrimidinyl, pyrazinyl, triazinyl, pyrrolyl, pyrazolyl, imidazolyl, (1,2,3)-triazolyl, and (1,2,4)-triazolyl, tetrazolyl, furanyl, thiophenyl, isoxazolyl, thiazolyl, oxazolyl, etc.

Unless otherwise specified as “substituted or unsubstituted”, the groups in the present invention can be substituted with substituents selected from the group consisting of: halogen, cyano, nitro, hydroxyl, amino, C₁-C₆ alkyl-amino, C₁-C₆ alkyl, C₂-C₆ alkenyl, C₂-C₆ alkynyl, C₁-C₆ alkoxy, C₁-C₆ haloalkyl, C₂-C₆ haloalkenyl, C₂-C₆ haloalkynyl, C₁-C₆ haloalkoxy, allyl, benzyl, C₆-C₁₂ aryl, C₁-C₆ alkoxy-C₁-C₆ alkyl, C₁-C₆ alkoxy-carbonyl, phenoxycarbonyl, C₂-C₆ alkynyl-carbonyl, C₂-C₆ alkynyl-carbonyl, C₃-C₆ cycloalkyl-carbonyl, and C₁-C₆ alkyl-sulfonyl, etc.

As used herein, “halogen” or “halogen atom” refers to F Cl, Br, and I. More preferably, the halogen or halogen atom is selected from F, Cl, and Br. “Halogenated” refers to being substituted with the atoms selected from F, Cl, Br, and I.

Unless otherwise specified, the structural formulas described in the present invention are intended to include all isomeric forms (such as enantiomers, diastereomers, and geometric isomers (or conformational isomers)), such as containing asymmetric center with R or S configuration, double bond with (Z) or (E) isomer, etc. Therefore, individual stereoisomers or mixtures of enantiomers, diastereomers, or geometric isomers (or conformational isomers) of the compound of the present invention are within the scope of the present invention.

As used herein, the term “tautomer” refers to structural isomers with different energy that can exceed a low energy barrier and thus transform into each other. For example, proton tautomers (i.e. proton shift) involve interconversion through proton transfer, such as ¹H indazole and 2H indazole. Valence tautomers involve interconversion through recombination of some bonding electrons.

As used herein, the term “solvate” refers to a coordination compound formed by the coordination of a compound of the present invention with solvent molecules in a specific ratio.

As used herein, the term “hydrate” refers to a coordination compound formed by the coordination of a compound of the present invention with water.

### Active Ingredient

The present invention provides an active ingredient that can effectively inhibit RSK. The active ingredient is a compound represented by formula (I), which can effectively prevent, treat, and/or alleviate RSK related diseases.

Experiments have shown that the active ingredient of the present invention can effectively inhibit RSK kinase protein, thereby preventing, treating, and/or alleviating RSK related diseases.

It should be understood that the active ingredients of the present invention include compounds represented by formula (I), or a pharmaceutically acceptable salt, or a prodrug thereof. It should be understood that the active ingredients of the present invention also include crystalline forms, amorphous compounds, and deuterated compounds of the compound of formula (I).

The term “pharmaceutically acceptable salt” refers to a salt, that is suitable for use as a drug, formed by the compound of the present invention with an acid or base. Pharmaceutically acceptable salts include inorganic salts and organic salts. A preferred type of salt is the salt formed by the compound of the present invention with an acid. The acids suitable for salt formation include but are not limited to inorganic acids such as hydrochloric acid, hydrobromic acid, hydrofluoric acid, sulfuric acid, nitric acid, phosphoric acid, etc; the organic acids such as formic acid, acetic acid, trifluoroacetic acid, propionic acid, oxalic acid, malonic acid, succinic acid, fumaric acid, maleic acid, lactic acid, malic acid, tartaric acid, citric acid, picric acid, benzoic acid, methanesulfonic acid, ethanesulfonic acid, p-toluenesulfonic acid, benzenesulfonic acid, naphthalene sulfonic acid, etc; and amino acids such as proline, phenylalanine, aspartic acid, glutamic acid, etc. The another preferred type of salt is the salt formed by the compound of the present invention with a base, such as alkali metal salts (e.g. sodium or potassium salts), alkaline earth metal salts (e.g. magnesium or calcium salts), ammonium salts (e.g. lower alkyl alcohol ammonium salts and other pharmaceutically acceptable ammonium salts), such as methylamine salts, ethylamine salts, propylamine salts, dimethylamine salts, trimethylamine salts, diethylamine salts, triethylamine salts, tert-butylamine salts, ethylenediamine salts, hydroxyethyl amine salts, dihydroxyethylamine salts, and amine salts formed by morpholine, piperazine, and lysine, respectively.

### Pharmaceutical Composition and Mode of Administration

Due to the excellent RSK kinase inhibitory activity of the compounds of the present invention, the compounds of the present invention, and various crystal forms, pharmaceutically acceptable inorganic or organic salts, hydrates or solvates thereof, as well as pharmaceutical compositions containing the compounds of the present invention as the main active ingredient, can be used for the prevention, treatment and/or alleviation of RKS related diseases, such as for the treatment of cancer.

The pharmaceutical composition of the present invention comprises a safe and effective dosage of the compound of the present invention and a pharmaceutically acceptable excipient or carrier. The term “safe and effective dosage” refers to the amount of a compound that is sufficient to significantly improve the condition without causing serious side effects. Generally, pharmaceutical composition contains 1-2000 mg compound of the present invention per dose, and more preferably 10-200 mg compound of the present invention per dose. Preferably, the “dose” is a capsule or tablet.

“Pharmaceutically acceptable carrier” refers to one or more compatible solid or liquid fillers or gel substances, which are suitable for human use and must have sufficient purity and low toxicity. “Compatibility” refers to that each component in the composition can be blended with the compound of the present invention and with each other without significantly reducing the efficacy of the compound. Examples of pharmaceutically acceptable carriers include cellulose and derivatives thereof (such as sodium carboxymethyl cellulose, sodium ethyl cellulose, cellulose acetate, etc.), gelatin, talc, solid lubricants (such as stearic acid, magnesium stearate), calcium sulfate, vegetable oils (such as soybean oil, sesame oil, peanut oil, olive oil, etc.), polyols (such as propylene glycol, glycerol, mannitol, sorbitol, etc.), emulsifiers (such as Tween®), wetting agents (such as sodium dodecyl sulfate), coloring agents, flavoring agents, stabilizers, antioxidants, preservatives, pyrogen-free water, etc.

The administration method of the compound or pharmaceutical combination of the present invention is not particularly limited, and representative administration methods include (but are not limited to): oral, parenteral (intravenous, intramuscular, or subcutaneous).

Solid dosage forms for oral administration include capsules, tablets, pills, powders, and granules. In these solid dosage forms, the active compound is mixed with at least one conventional inert excipient (or carrier), such as sodium citrate or dicalcium phosphate, or with the following components: (a) fillers or compatibilizers, such as starch, lactose, sucrose, glucose, mannitol, and silica acid; (b) binders, such as hydroxymethylcellulose, alginate, gelatin, polyvinylpyrrolidone, sucrose, and arabic gum; (c) humectant, such as glycerin; (d) disinfectants, such as agar, calcium carbonate, potato starch or cassava starch, alginic acid, certain complex silicates, and sodium carbonate; (e) dissolution-retarding agents, such as paraffin wax; (f) absorption accelerators, such as quaternary amine compounds; (g) wetting agents, such as cetyl alcohol and glycerol monostearate; (h) adsorbents, such as kaolin; and (i) lubricants, such as talc, calcium stearate, magnesium stearate, solid polyethylene glycol, sodium dodecyl sulfate, or mixtures thereof. In capsules, tablets, and pills, the dosage forms may also contain buffering agents.

The solid dosage forms such as tablets, sugar pills, capsules, pills, and granules can be prepared using coating and shell materials, such as enteric coatings and other materials known in the art. They may contain opaque agents, and the active compound or compound in this composition can be released in a delayed way in a given portion of the digestive tract. Examples of usable embedding components are polymers and waxs. If necessary, the active compound can also form microcapsules with one or more above-mentioned excipients.

Liquid dosage forms for oral administration include pharmaceutically acceptable emulsions, solutions, suspensions, syrups or tinctures. In addition to active compound, liquid dosages may include inert diluents commonly used in this field, such as water or other solvents, solubilizers, and emulsifiers, such as ethanol, isopropanol, ethyl carbonate, ethyl acetate, propylene glycol, 1,3-butanediol, dimethylformamide, as well as oils, particularly cottonseed oil, peanut oil, corn germ oil, olive oil, castor oil, and sesame oil, or a combination thereof.

In addition to these inert diluents, the composition may also contain adjuvants, such as wetting agents, emulsifiers and suspensions, sweeteners, flavoring agents, and spices.

In addition to active compound, suspensions may contain suspending agents, such as ethoxylated isooctadecanol, polyoxyethylene sorbitol and dehydrated sorbitol esters, microcrystalline cellulose, methanol aluminum and agar, or a combination thereof.

The compositions for parenteral injection may include physiologically acceptable sterile aqueous or anhydrous solutions, dispersions, suspensions or emulisons, and sterile powders that can be re-dissolved into sterile injectable solutions or dispersions. The suitable aqueous and non-aqueous carriers, diluents, solvents, or excipients include water, ethanol, polyols, and a suitable combination thereof.

The compound of the present invention can be administered alone or in combination with other pharmaceutically acceptable therapeutic agents.

When administered in combination, the pharmaceutical composition further comprises one or more (2, 3, 4, or more) other pharmaceutically acceptable therapeutic agents. The one or more other pharmaceutically acceptable therapeutic agents (2, 3, 4, or more) can be used simultaneously, separately, or sequentially with the compound of the present invention for the prevention, treatment, and/or alleviation of RSK mediated diseases.

When a pharmaceutical composition is used, a safe and effective dosage of the compound of the present invention is applied to a mammal (such as a human) in need of treatment, wherein the dosage of administration is a pharmaceutically effective dosage. For a person weighed 60 kg, the daily dosage is usually 1-2000 mg, preferably 20-500 mg. Of course, the specific dosage should also consider factors such as the route of administration and the patient's health condition, which are within the skill range of skilled physicians.

### Beneficial Effects

According to the embodiments of the present invention, the present invention provides RSK inhibitors with novel structure, excellent pharmacokinetic properties, and good efficacy or druggability, which can be used for treating RSK related diseases and conditions effectively.

The compound of the present invention has a good inhibitory effect against RSK and good in vitro efficacy. In addition, the results of mouse experiments showed that the compounds of the present invention exhibited excellent pharmacokinetic properties and good druggability.

The present invention will be further illustrated below in conjunction with the examples. The technicians in this field will understand that the following examples are only used to illustrate the present invention and should not be considered as limiting the scope of the present invention. The specific technology or conditions that are not specified in the examples are performed according to the technology or conditions described in the literature in this field or according to the product manual. The reagents or instruments used without specifying the manufacturer are conventional products that can be obtained through commercial purchase.

Unless otherwise specified, the structures of the compounds of the present invention are determined by nuclear magnetic resonance (NMR) and/or mass spectrometry (MS). The unit of NMR shift is 10⁻⁶ (ppm). The solvents used for NMR determination are deuterated dimethyl sulfoxide, deuterated chloroform, deuterated methanol, etc, and the internal standard is tetramethylsilane (TMS).

The abbreviation definition of the present invention is as follows:
M: Molar concentration, for example, 1M hydrochloric acid represents 1 mol/L hydrochloric acid solution
HATU: O-(7-azabenzotriazole-1-yl)-N,N,N,N-tetramethylurea hexafluorophosphate
DIPEA: It can also be written as DIEA, diisopropylethylamine, as well as N,N-diisopropylethylamine
NIS: N-iodobutyrimide
AIBN: Azobisisobutyronitrile
DMF: N,N-dimethylformamide
THF: Tetrahydrofuran
PE: Petroleum ether
DBAD: Azodicarboxylate dibenzyl ester
LC-MS: Liquid chromatography-mass spectrometry
DMSO: Dimethyl sulfoxide
DTT: Dithiothreitol
ATP: Adenosine triphosphate
Xantphos: 4,5-diphenylphosphine-9,9-dimethyloxaanthracene
PyBOP: 1H-benzotriazole-1-yloxytriphyrrolidinyl hexafluorophosphate
TLC: Thin layer chromatography
IC₅₀: Half inhibitory concentration refers to the concentration at which half of the maximum inhibitory effect is achieved.

Unless otherwise indicated, the compounds listed herein are named and numbered using ChemBioDraw Ultra 14.0.

### Intermediate 13a Diethyl (R)-1-(1-(tert-butoxycarbonyl) amino) propan-2-yl)-7-fluoro-1H-indole-2,6-dicarboxylate

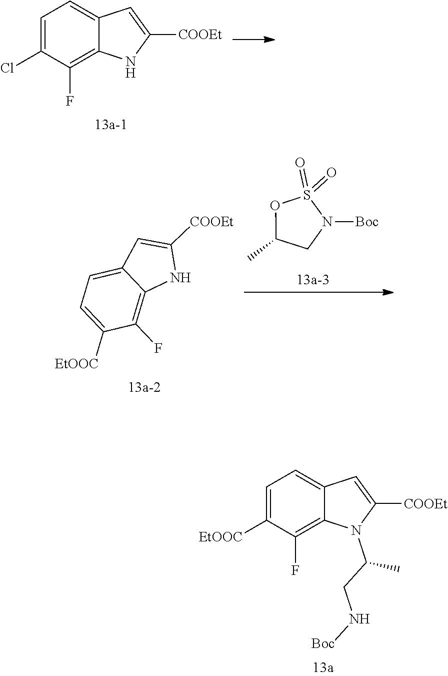 [SMILES: CCOC(=O)c1cc2ccc(Cl)c(F)c2[nH]1 ; CCOC(=O)c1cc2ccc(C(=O)OCC)c(F)c2[nH]1 ; C[C@H]1CN(C(=O)OC(C)(C)C)S(=O)(=O)O1 ; CCOC(=O)c1ccc2cc(C(=O)OCC)n([C@H](C)CNC(=O)OC(C)(C)C)c2c1F]

### Step 1 diethyl 7-fluoro-1H-indole-2,6-dicarboxylate 13a-2

To a 50 mL round bottom flask, Compound 13a-1 (564 mg, 2.34 mmol), palladium chloride (13 mg, 0.059 mmol), 1,1′-bis(diphenylphosphine) ferrocene (65 mg, 0.12 mmol), sodium acetate (576 mg, 7.02 mmol), ethanol (10 mL), DMF (10 mL) was added in sequence, after replaced with CO three times, it was heated up to 110° C. under 300 psi pressure and reacted overnight. After monitoring the completion of the reaction, it was filtered through diatomaceous earth, extracted with ethyl acetate, washed with saturated sodium chloride solution, dried over anhydrous sodium sulfate, and purified by normal phase chromatography to obtain compound 13a-2 (white solid, 316 mg).

MS (ESI) m/z 280.2 [M+H]⁺

¹H NMR (500 MHz, CDCl₃) δ 9.28 (s, 1H), 7.66 (dd, J=8.5, 6.3 Hz, 1H), 7.46 (d, J=8.5 Hz, 1H), 7.24-7.22 (m, 1H), 4.44 (dq, J=8.8, 7.2 Hz, 4H), 1.43 (td, J=7.1, 5.3 Hz, 6H).

### Step 2 diethyl (R)-1-(1-(tert-butoxycarbonyl) amino) propan-2-yl)-7-fluoro-1H-indole-2,6-dicarboxylate 13a

In a three necked bottle, under the ice bath conditions, NaH (60%, 54 mg) was dispersed in anhydrous DMF (2 mL), and compound 13a-1 (316 mg, 1.13 mmol) in DMF solution (5 mL) was added dropwise, and stirred for 30 minutes, and then DMF solution (5 mL) of (S)-5-methyl-1,2,3-oxothiazolidine-3-carboxylic acid tert-butyl-2,2-dioxide (13a-3) (295 mg, 1.24 mmol) was added slowly dropwise. After completion, the mixture was moved to room temperature and reacted overnight, after monitoring the completion of the reaction, it was quenched with water, extracted with ethyl acetate, washed with saturated sodium chloride solution, dried over anhydrous sodium sulfate, purified by normal phase chromatography to obtain compound 13a (white solid, 270 mg).

MS (ESI) m/z 437.2 [M+H]⁺

### Example 1 (R)—N-(1-(4-(dimethylphosphoryl) benzyl)-1H-pyrazol-4-yl)-9-methyl-6-oxo-6,7,8,9-tetrahydropyridino[3′, 2′: 4,5]pyrrolo [1,2-a]pyrazine-2-carboxamide

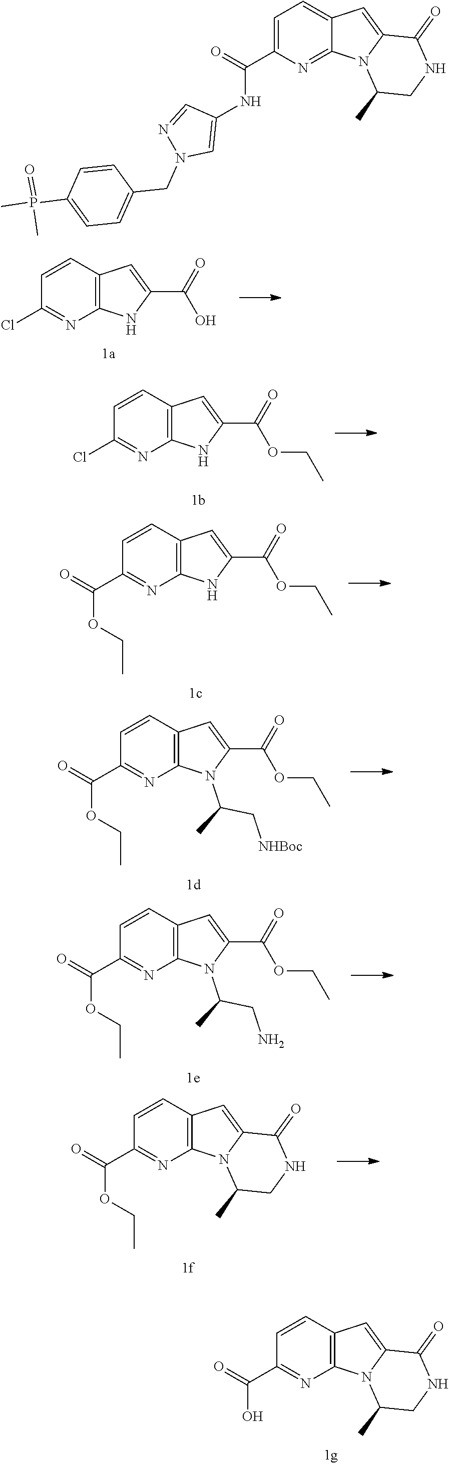

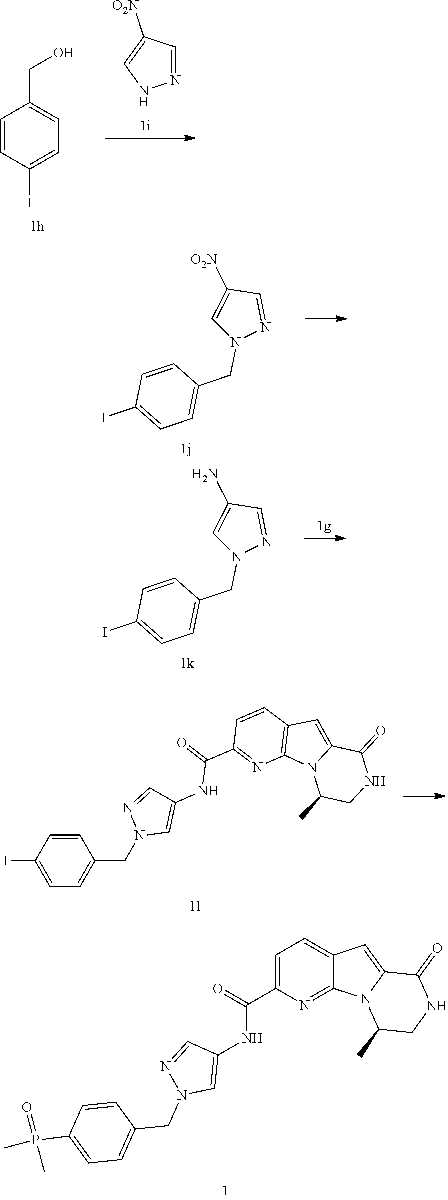 [SMILES: OCc1ccc(I)cc1 ; O=[N+]([O-])c1cn[nH]c1 ; O=[N+]([O-])c1cnn(Cc2ccc(I)cc2)c1 ; Nc1cnn(Cc2ccc(I)cc2)c1 ; C[C@@H]1CNC(=O)c2cc3ccc(C(=O)Nc4cnn(Cc5ccc(I)cc5)c4)nc3n21 ; C[C@@H]1CNC(=O)c2cc3ccc(C(=O)Nc4cnn(Cc5ccc(P(C)(C)=O)cc5)c4)nc3n21]

### Step 1 ethyl 6-chloro-1H-pyrrolo [2,3-b]pyridine-2-carboxylate 1b

Compound 1a (10.0 mmol, 1.96 g) was dissolved in dichloromethane (50 mL), thionyl chloride (20.0 mmol, 14.0 mL) and 4 drops DMF were added in sequence at room temperature, and then it was heated up to 50° C. and reacted for 5 hours. The reaction mixture was cooled down to room temperature and added dropwise slowly to ethanol (100 mL), stirred for 1 hour, and the mixture was concentrated under reduced pressure to obtain a crude product, and the crude product was purified by column chromatography (PE/EtOAc=5/1) to obtain compound 1b (1.70 g), a white solid.

MS (ESI) m/z 225.2 [M+H]⁺.

¹H NMR (500 MHz, DMSO-d6) δ 12.73 (s, 1H), 8.16 (d, J=8.3 Hz, 1H), 7.22 (dd, J=8.3, 1.8 Hz, 1H), 7.20-7.18 (m, 1H), 4.34 (q, J=7.1 Hz, 2H), 1.33 (t, J=7.1 Hz, 3H).

### Step 2 diethyl 1H-pyrrolo [2,3-b]pyridine-2,6-dicarboxylate 1c

To a high-pressure reaction tube was added compound 1b (10.0 mmol, 2.24 g), sodium acetate (30.0 mmol, 2.46 g), and 1,1′-biphenylphosphine ferrocene (0.50 mmol, 270 mg), palladium acetate (0.25 mmol, 5.60 mg), and ethanol (50 mL) in sequence. Under the pressure of CO (250 psi), it was heated up to 110° C. and reacted for 16 hours. After monitoring the completion of the reaction, it was cooled down to room temperature, filtered through diatomaceous earth, extracted with ethyl acetate (50 mL×3), and the organic phases were combined, washed with saturated saline water (50 mL), and concentrated under reduced pressure to obtain a crude product, and the crude product was purified by column chromatography (PE/EtOAc=5/1) to obtain compound 1c (1.50 g), a white solid.

MS (ESI) m/z 263.2 [M+H]⁺.

¹H NMR (500 MHz, DMSO-d6) δ 12.94 (s, 1H), 8.28 (d, J=8.2 Hz, 1H), 7.88 (d, J=8.2 Hz, 1H), 4.36 (p, J=7.0 Hz, 4H), 1.35 (q, J=7.0 Hz, 6H).

### Step 3 diethyl (R)-1-(1-(tert-butoxycarbonyl) amino) propan-2-yl)-¹H pyrrolo [2,3-b]pyridine-2,6-dicarboxylate 1d

In a 50 mL three necked bottle, sodium hydride (2.60 mmol, 104 mg, 60% in mineral oil) was dissolved in DMF (3 mL) at 0° C., compound 1c (2.00 mmol, 524 mg) in DMF solution (5 mL) was slowly added dropwise, and stirred for 1 hour, and then DMF solution (5 mL) of tert-butyl (S)-5-methyl-1,2,3-oxathiazolidine-3-carboxylate 2,2-dioxide (2.40 mmol, 540 mg) was added dropwise slowly, and the reaction mixture reacted at room temperature for additional 16 hours. After TLC monitored the completion of the reaction, the reaction was quenched with saturated sodium bicarbonate solution (20 mL), extracted with ethyl acetate (50 mL×3), and the organic phases were combined, washed with saturated saline solution (50 mL), and concentrated under reduced pressure to obtain a crude product, and the crude product was purified by column chromatography (PE/EtOAc=3/1) to obtain compound 1d (818 mg).

MS (ESI) m/z 420.3 [M+H]⁺.

¹H NMR (500 MHz, CDCl₃) δ 8.10 (d, J=8.2 Hz, 1H), 7.99 (d, J=8.2 Hz, 1H), 7.30 (s, 1H), 6.19 (s, 1H), 5.92-5.84 (m, 1H), 4.59-4.46 (m, 2H), 4.43 (q, J=7.1 Hz, 2H), 4.28 (dt, J=14.6, 8.4 Hz, 1H), 3.59 (dt, J=14.7, 3.5 Hz, 2H), 1.64 (s, 3H), 1.49 (t, J=7.1 Hz, 3H), 1.47-1.41 (m, 12H)).

### Step 4 diethyl (R)-1-(1-aminopropan-2-yl)-1H pyrrolo [2,3-b]pyridine-2,6-dicarboxylate 1e

Trifluoroacetic acid (1 mL) was added dropwise to the dichloromethane solution (5 mL) of compound 1d (1.95 mmol, 818 mg), and stirred at room temperature for 3 hours. After TLC monitored the completion of the reaction, the reaction mixture was concentrated under reduced pressure, then saturated sodium bicarbonate (50 mL) was added, and extracted with ethyl acetate (50 mL×3). The organic phase was concentrated under reduced pressure to obtain a crude product, and the crude product was purified by column chromatography (PE/EtOAc=1/1) to obtain compound 1e (498 mg).

MS (ESI) m/z 320.2 [M+H]⁺.

¹H NMR (500 MHz, CDCl₃) δ 8.20 (d, J=8.2 Hz, 1H), 8.03 (d, J=8.2 Hz, 1H), 7.35 (s, 1H), 6.28 (p, J=7.0 Hz, 1H), 4.51-4.40 (m, 4H), 3.85 (dd, J=14.6, 6.6 Hz, 1H), 3.58 (d, J=14.5 Hz, 1H), 1.71 (d, J=7.2 Hz, 3H), 1.47 (t, J=7.1 Hz, 3H), 1.43 (t, J=7.1 Hz, 3H).

### Step 5 ethyl (R)-9-methyl-6-oxo-6,7,8,9-tetrahydropyridino [3′, 2′: 4,5]pyrrolo [1,2-a]pyrazine-2-carboxylate if

Compound 1e (1.03 mmol, 330 mg) was dissolved in ethanol (10 mL) and placed in a 50 mL round bottom flask, and then potassium carbonate (3.09 mmol, 428 mg) was added and heated up to 60° C. to react for 3 hours. After TLC monitored the completion of the reaction, the reaction mixture was concentrated under reduced pressure and extracted with ethyl acetate (50 mL×3). The organic phases were combined, washed with saturated saline solution (50 mL), and concentrated to obtain a crude compound, and the crude product was purified by column chromatography (PE/EtOAc=1/1) to obtain compound if (225 mg).

MS (ESI) m/z 274.2 [M+H]⁺.

¹H NMR (500 MHz, CDCl₃) δ 8.13 (d, J=8.2 Hz, 1H), 7.98 (d, J=8.2 Hz, 1H), 7.26 (s, 1H), 6.42 (s, 1H), 5.31 (dt, J=12.0, 6.2 Hz, 2H), 4.49 (qd, J=7.1, 2.7 Hz, 2H), 4.03 (dd, J=12.7, 4.4 Hz, 1H), 3.53 (dd, J=12.6, 5.1 Hz, 1H), 1.65 (s, 2H), 1.55 (d, J=6.6 Hz, 3H), 1.46 (t, J=7.1 Hz, 3H).

### Step 6 (R)-9-methyl-6-oxo-6,7,8,9-tetrahydropyridino [3′, 2′: 4,5]pyrrolo [1,2-a]pyrazine-2-carboxylic acid 1g

Lithium hydroxide aqueous solution (1.2 mL, 2.0 M) was added dropwise slowly to a 1,4-dioxane solution (6 mL) of compound if (1.31 mmol, 362 mg), and stirred at room temperature for 1 hour. After TLC monitored the completion of the reaction, the reaction mixture was concentrated under reduced pressure until a white solid was precipitated, and then it was filtered to obtain compound 1g (148 mg).

MS (ESI) m/z 246.3 [M+H]⁺.

¹H NMR (500 MHz, DMSO-d6) δ 8.26 (d, J=8.2 Hz, 1H), 7.88 (d, J=8.2 Hz, 1H), 7.12 (s, 1H), 5.09-5.03 (m, 1H), 3.87 (dd, J=13.1, 4.7 Hz, 1H), 3.42 (ddd, J=13.1, 5.1, 1.3 Hz, 2H), 1.39 (d, J=6.6 Hz, 3H).

### Step 7 1-(4-iodophenyl)-4-nitro-1H-pyrazole 1j

4-iodobenzenol (23.1 mmol, 5.40 g) was dissolved in dichloromethane (50 mL), and thionyl chloride (34.7 mmol, 4.10 g) was added slowly to react at room temperature for 4 hours. The reaction mixture was rotary dried to remove the solvent and dissolved in acetone (80 mL), then 4-nitro-1H-pyrazole (32.3 mmol, 3.65 g), potassium carbonate (46.5 mmol, 6.42 g), and sodium iodide (4.66 mmol, 0.0 g) were added and heated to reflux for 2 hours. The reaction mixture was concentrated and extracted with ethyl acetate (50 mL×3). The organic phases were combined, washed with saturated saline solution. The organic phase was concentrated under reduced pressure to obtain a crude product, and the crude product was purified by column chromatography (PE/EtOAc=2/1) to obtain compound 1j (6.21 g).

MS (ESI) m/z 330.0 [M+H]⁺.

¹H NMR (500 MHz, CDCl₃) δ 8.09 (s, 1H), 8.07 (s, 1H), 7.74 (d, J=8.3 Hz, 2H), 7.04 (d, J=8.3 Hz, 2H), 5.25 (s, 2H).

### Step 8 1-(4-iodophenyl)-1H-pyrazole-4-amine 1k

Compound 1j (8.27 mmol, 2.72 g) was dissolved in ethanol (30 mL), and water (30 mL), ammonium chloride (16.6 mmol, 0.88 g), iron powder (246 mmol, 1.38 g) were added in sequence, and then heated up to 70° C. to react for 2 hours. After TLC monitored the completion of the reaction, it was cooled down to room temperature. The reaction mixture was filtered through diatomaceous earth, and the filter cake was extracted with ethyl acetate (50 mL), the filtrate was extracted with ethyl acetate (50 mL×3), washed with saturated saline, and the organic phase was concentrated under reduced pressure to obtain a crude product, and the crude product was purified by column chromatography (PE/EtOAc=1/1) to obtain compound 1k (1.61 g).

MS (ESI) m/z 300.10 [M+H]⁺.

¹H NMR (500 MHz, CDCl₃) δ 7.66-7.63 (m, 2H), 7.19 (d, J=0.7 Hz, 1H), 6.97 (d, J=0.8 Hz, 1H), 6.92 (d, J=8.4 Hz, 2H), 5.11 (s, 2H).

### Step 9 (R)—N-(1-(4-iodophenyl)-1H-pyrazol-4-yl)-9-methyl-6-oxo-6,7,8,9-tetrahydropyridino [3′, 2′: 4,5]pyrrolo [1,2-a]pyrazine-2-carboxamide 1l

Compound 1k (0.40 mmol, 120 mg) and compound 1g (0.42 mmol, 108 mg) were dissolved in DMF (10 mL), then DIPEA (0.64 mmol, 0.11 mL) and HATU (0.64 mmol, 242 mg) were added at room temperature, and finally the reaction mixture reacted at room temperature for 24 hours. After TLC monitored the completion of the reaction, it was diluted with water (30 mL), extracted with ethyl acetate (30 mL×3), and the organic phases were combined, washed with saturated saline solution, and concentrated under reduced pressure to obtain a crude product, and the crude product was purified by column chromatography (PE/EtOAc=1/1) to obtain compound 1l (157 mg).

MS (ESI) m/z 527.3 [M+H]⁺.

¹H NMR (500 MHz, CDCl₃) δ 9.65 (s, 1H), 8.20 (d, J=8.3 Hz, 2H), 8.13 (d, J=8.2 Hz, 1H), 7.68 (d, J=8.4 Hz, 2H), 7.67-7.66 (m, 1H), 7.29 (s, 1H), 7.01 (d, J=8.4 Hz, 2H), 6.21 (d, J=4.9 Hz, 1H), 5.25 (s, 2H), 5.24-5.19 (m, 1H), 4.09 (dd, J=12.6, 4.4 Hz, 1H), 3.57 (ddd, J=12.6, 5.1) 1.4 Hz, 1H), 1.60 (d, J=6.7 Hz, 3H).

### Step 10 (R)—N-(1-(4-(dimethylphosphoryl) benzyl)-1H-pyrazol-4-yl)-9-methyl-6-oxo-6,7,8,9-tetrahydropyridino [3′, 2′: 4,5]pyrrolo [1,2-a]pyrazine-2-carboxamide 1

Compound 1l (0.16 mmol, 84.0 mg) was dissolved in ethanol (3 mL), and then tetratriphenylphosphine palladium (0.016 mmol, 19.0 mg), dimethyl phosphate oxide (0.19 mmol, 16 mg), and triethylamine (0.32 mmol, 45 L) were added, evacuated and exchanged with argon gas three times, and then heated up to 100° C. to react for 6 hours. After TLC monitored the completion of the reaction, the reaction mixture was cooled down to room temperature, and filtered through diatomaceous earth. The filter cake was washed with ethyl acetate (50 mL), the filtrate was extracted with ethyl acetate (50 mL×3), the organic phase was concentrated under reduced pressure to obtain a crude product, and the crude product was purified by column chromatography (PE/EtOAc=1/1) to obtain compound 1 (29 mg).

MS (ESI) m/z 477.2 [M+H]⁺.

¹H NMR (500 MHz, CD3OD) δ 8.28 (s, 1H), 8.27 (d, J=8.3 Hz, 1H), 8.01 (d, J=8.2 Hz, 1H), 7.88 (d, J=0.5 Hz, 1H), 7.79 (dd, J=11.7, 8.3 Hz, 1H), 7.44 (dd, J=8.2, 2.3 Hz, 1H), 7.24 (s, 1H), 5.45 (s, 2H), 5.39-5.32 (m, 1H), 4.00 (dd, J=13.1, 4.5 Hz, 1H), 3.57 (dd, J=13.1, 1.3 Hz, 1H), 1.78 (s, 3H), 1.76 (s, 3H), 1.51 (d, J=6.6 Hz, 3H).

### Example 2 (R)—N-(1-(4-((dimethyl (oxo)-λ⁶-sulfanylidene) amino) benzyl)-1H-pyrazol-4-yl)-9-methyl-6-oxo-6,7,8,9-tetrahydropyridino [3′,2′: 4,5]pyrrolo [1,2-a]pyrazine-2-carboxamide

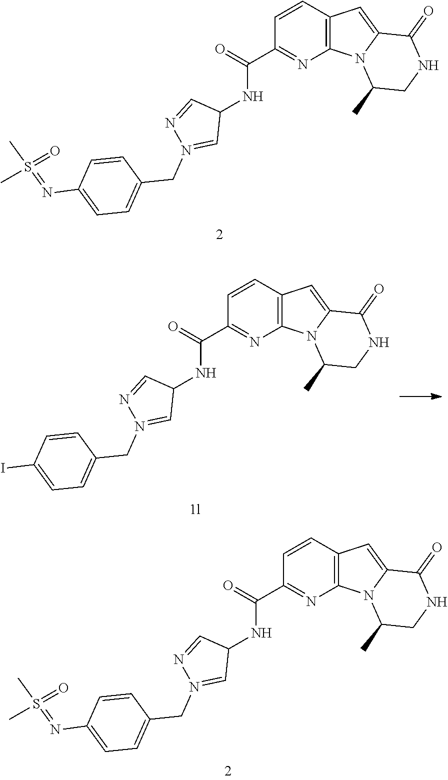

Compound 1l (0.5 mmol, 260 mg) was dissolved in 1,4-dioxane (3 mL), and dimethyl sulfinyl imide (0.60 mmol, 60.0 mg), 4,5-diphenylphosphine-9,9-dimethyloxanthracene (0.10 mmol, 50.0 mg), Pd₂(dba)₃ (0.05 mmol, 46.0 mg), and cesium carbonate (0.75 mmol, 240 mg) were added at room temperature, evacuated and exchanged with argon gas three times, and then heated up to 100° C. to react for 6 hours. After TLC monitored the completion of the reaction, the reaction mixture was cooled down to room temperature, and filtered through diatomaceous earth. The filter cake was washed with ethyl acetate (50 mL), and the filtrate was extracted with ethyl acetate (50 mL×3). The organic phase was concentrated under reduced pressure to obtain a crude product, and the crude product was purified by column chromatography (PE/EtOAc=1/1) to obtain compound 2 (50 mg), a light yellow solid.

MS (ESI) m/z 492.2 [M+H]⁺.

¹H NMR (500 MHz, DMSO-d6) δ 10.64 (s, 1H), 8.35 (d, J=4.9 Hz, 1H), 8.29 (d, J=8.2 Hz, 1H), 8.17 (s, 1H), 7.91 (d, J=8.2 Hz, 1H), 7.77 (s, 1H), 7.14 (d, J=7.1 Hz, 3H), 6.90 (d, J=8.4 Hz, 2H), 5.31-5.25 (m, 1H), 5.22 (s, 2H), 3.91 (dd, J=13.0, 4.3 Hz, 1H), 3.47 (dd, J=12.2, 5.2 Hz), 1H), 3.20 (s, 6H), 1.42 (d, J=6.6 Hz, 3H).

### Example 3 (9R)-9-methyl-N-(1-(4-(S-methylsulfinimide) benzyl)-1H-pyrazol-4-yl)-6-oxo-6,7,8,9-tetrahydropyridino [3′,2]: 4,5]pyrrolo [1,2-a]pyrazine-2-carboxamide

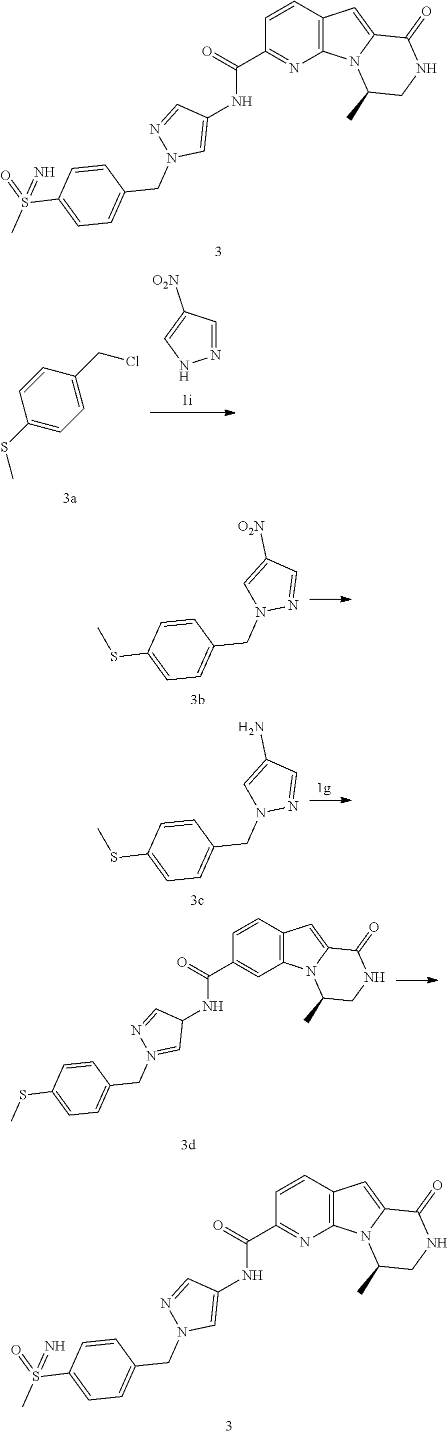

### Step 1-(4-(methylthio) benzyl)-4-nitro-1H-pyrazole 3b

Compound 3a (4.50 g, 26.2 mmol) was dissolved in acetone (80 mL), 4-nitro-1H-pyrazole (31.2 mmol, 3.54 g) and potassium carbonate (52.2 mmol, 7.20 g) were added, and heated up to 60° C. to react for 4 hours. After TLC monitored the completion of the reaction, it was concentrated under reduced pressure, and the obtained solid was diluted with added water (100 mL), extracted with ethyl acetate (100 mL×3). The organic phases were combined, washed with saturated saline and concentrated under reduced pressure to obtain a crude product, and the crude product was purified by column chromatography (PE/EtOAc=10/1) to obtain compound 3b (5.20 g), a white solid.

MS (ESI) m/z 250.2 [M+H]⁺.

¹H NMR (500 MHz, CDCl₃) δ 8.08 (s, 1H), 8.03 (s, 1H), 7.26 (d, J=8.0 Hz, 2H), 7.22 (d, J=8.3 Hz, 2H), 5.25 (s, 2H), 2.48 (s, 3H).

### Step 2 1-(4-(methylthio) benzyl)-1H-pyrazole-4-amine 3c

Compound 3b (7.90 mmol, 1.97 g) was dissolved in tetrahydrofuran (50 mL), and 0.20 g palladium on carbon (5%, wet basis) was added, and then reacted for 8 hours in H₂ (15 psi) environment. After TLC monitored the completion of the reaction completion, the reaction mixture was filtered through diatomaceous earth, and the filter cake was washed with tetrahydrofuran (100 mL). The filtrate was spun dried to remove solvent to obtain a crude product, and the crude product was purified by column chromatography (PE/EtOAc=0/1) to obtain compound 3c (1.55 g).

MS (ESI) m/z 220.2 [M+H]⁺.

¹H NMR (500 MHz, CDCl₃) δ 7.20 (d, J=8.3 Hz, 2H), 7.17 (s, 1H), 7.11 (d, J=8.2 Hz, 2H), 5.12 (s, 2H), 2.45 (s, 3H).

### Step 3 (R)-9-methyl-N-(1-(4-(methylthio) benzyl)-1H-pyrazol-4-yl)-6-oxo-6,7,8,9-tetrahydropyridino [3′, 2′: 4,5]pyrrolo [1,2-a]pyrazine-2-carboxamide 3d

Compound 3c (1.50 mmol, 329 mg) and compound 1g (1.65 mmol, 419 mg) were dissolved in DMF (5 mL), then DIPEA (2.25 mmol, 0.40 mL) and HATU (2.25 mmol, 855 mg) were added, and the reaction mixture reacted at room temperature for 24 hours. After TLC monitored the completion of the reaction, it was diluted with water (50 mL), extracted with ethyl acetate (50 mL×4), the organic phases were combined, washed with saturated saline solution, and concentrated under reduced pressure to obtain a crude product, and the crude product was purified by column chromatography (PE/EtOAc=1/1) to obtain compound 3d (408 mg).

MS (ESI) m/z 447.1 [M+H]⁺.

¹H NMR (500 MHz, DMSO-d6) δ 10.65 (s, 1H), 8.36 (d, J=4.8 Hz, 1H), 8.30 (d, J=8.2 Hz, 1H), 8.21 (s, 1H), 7.91 (d, J=8.2 Hz, 1H), 7.78 (s, 1H), 7.27-7.22 (m, 4H), 7.13 (s, 1H), 5.29 (m, 3H), 3.91 (dd, J=13.0, 4.4 Hz, 1H), 3.48 (dd, J=12.8, 5.0 Hz, 1H), 2.46 (s, 3H), 1.42 (d, J)=6.6 Hz, 3H).

### Step 4 (9R)-9-methyl-N-(1-(4-(S-methylsulfinimide) benzyl)-1H-pyrazol-4-yl)-6-oxo-6,7,8,9-tetrahydropyridino [3′, 2]: 4,5]pyrrolo [1,2-a]pyrazine-2-carboxamide 3

Compound 3d (0.50 mmol, 223 mg) was dissolved in DMF (5 mL), and then ammonium aminoacetate (1.00 mmol, 78.0 mg) and iodobenzene diacetate (1.25 mmol, 403 mg) were added in sequence at room temperature, and the reaction mixture reacted at room temperature for 2 hours. After TLC monitored the completion of the reaction, it was diluted with added water (30 mL), extracted with ethyl acetate (30 mL×3), the organic phases were combined, washed with saturated saline solution, and concentrated under reduced pressure to obtain a crude product, and the crude product was purified by column chromatography (PE/EtOAc=1/1) to obtain compound 3 (55 mg).

MS (ESI) m/z 478.2 [M+H]⁺.

¹H NMR (500 MHz, DMSO-d6) δ 10.68 (s, 1H), 8.35 (d, J=5.0 Hz, 1H), 8.30 (d, J=9.0 Hz, 2H), 7.91 (dd, J=8.2, 3.5 Hz, 3H), 7.82 (s, 1H), 7.44 (d, J=8.3 Hz, 2H), 7.13 (s, 1H), 5.47 (s, 2H), 5.28 (m, 1H), 3.91 (dd, J=12.9, 4.4 Hz, 1H), 3.48 (dd, J=12.6, 4.6 Hz, 1H), 3.05 (s, 3H), 1.43 (s, 2H) d, J=6.6 Hz, 3H).

### Example 4 (R)—N-(1-(2-(2-hydroxypropan-2-yl) pyridin-4-yl) methyl)-1H-pyrazol-4-yl)-9-methyl-6-oxo-6,7,8,9-tetrahydropyridino [3′, 2′: 4,5]pyrrolo [1,2-a]pyrazine-2-carboxamide

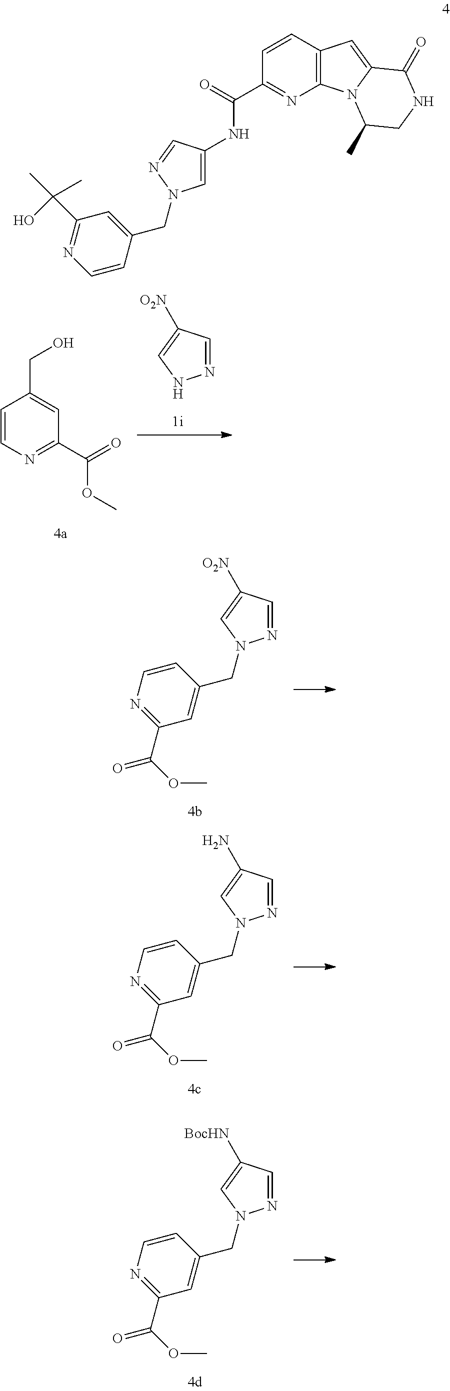 [SMILES: C[C@@H]1CNC(=O)c2cc3ccc(C(=O)Nc4cnn(Cc5ccnc(C(C)(C)O)c5)c4)nc3n21 ; COC(=O)c1cc(CO)ccn1 ; O=[N+]([O-])c1cn[nH]c1 ; COC(=O)c1cc(Cn2cc([N+](=O)[O-])cn2)ccn1 ; COC(=O)c1cc(Cn2cc(N)cn2)ccn1 ; COC(=O)c1cc(Cn2cc(NC(=O)OC(C)(C)C)cn2)ccn1]

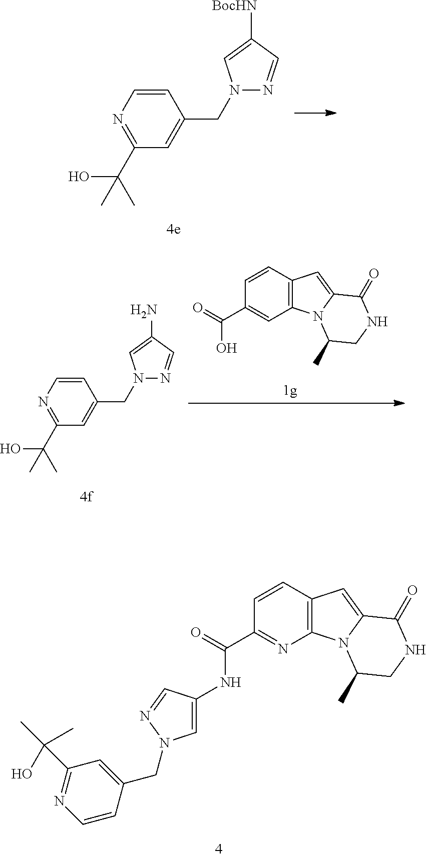

### Step 1 methyl 5-(4-nitro-1H-pyrazol-1-yl) methyl) pyridinecarboxylate 4b

Compound 4a (4.00 g, 23.95 mmol), compound 1i (3.14 g, 27.78 mmol), and triphenylphosphine (6.90 g, 26.33 mmol) were added in tetrahydrofuran (50 mL), and replaced with argon gas three times, cooled down to 0° C., di-tert-butyl azodicarboxylate (6.03 g, 27.33 mmol) was added, and the mixture reacted at room temperature for 24 hours. After TLC monitored the completion of the reaction, it was diluted with added water (100 mL) and extracted with ethyl acetate (100 mL×3). The organic phases were combined, washed with saturated saline, and concentrated under reduced pressure to obtain a crude product, and the crude product was purified by column chromatography (PE/EtOAc=2/1) to obtain compound 4b (5.9 g).

MS (ESI) m/z 263.1 [M+H]⁺.

¹H NMR (500 MHz, DMSO-d6) δ 3.85 (3H, s), 5.57 (2H, s), 7.47 (1H, dd, J=1.5 Hz, J=5 Hz), 7.95 (1H, s), 8.33 (1H, S), 8.67 (1H, d, J=5 Hz), 9.09 (1H, s).

### Step 2 methyl 5-(4-amino-1H-pyrazol-1-yl) methyl) pyridinecarboxylate 4c

Compound 4b (5.72 mmol, 1.50 g) was dissolved in tetrahydrofuran (50 mL), and 0.3 g palladium on carbon (5%, wet basis) was added to react in H₂ (15 psi) environment for 5 hours. After TLC monitored the completion of the reaction, the reaction mixture was filtered through diatomaceous earth, the filter cake was washed with tetrahydrofuran (100 mL), and the filtrate was spun dried to obtain the crude compound 4c (1.04 g, crude), which was directly used for the next reaction step.

MS m/z (ESI): 233 [M+H]⁺.

### Step 3 methyl 5-((4-(tert-butoxycarbonyl) amino)-1H-pyrazol-1-yl) methyl) pyridinecarboxylate 4d

Compound 4c (4.46 mmol, 1.04 g) was dissolved in tetrahydrofuran (50 mL), DMAP (0.196 mmol, 22.0 mg) and di-tert-butyl dicarbonate (4.48 mmol, 0.99 g) were added at 0° C., and the reaction mixture reacted at room temperature for 14 hours. After TLC monitored the completion of the reaction, it was diluted with water (50 mL), extracted with ethyl acetate (50 mL×3), the organic phases were combined, washed with saturated saline solution, and concentrated under reduced pressure to obtain a crude product, and the crude product was purified by column chromatography (PE/EtOAc=1/1) to obtain compound 4d (0.70 g, yield: 47%).

MS(ESI) m/z 333.25 [M+H]⁺.

### Step 4 tert-butyl (1-(6-(2-hydroxypropan-2-yl) pyridin-3-yl) methyl)-1H-pyrazol-4-yl) carbamate 4e

Compound 4d (2.11 mmol, 700 mg) was dissolved in ultra dry tetrahydrofuran (20 mL), and a tetrahydrofuran solution of methyl magnesium bromide (8.42 mmol, 8.42 mL, 1.0 M) was added dropwise at −70° C., and the reaction mixture reacted at room temperature for 2 hours. The reaction was quenched with the added saturated ammonium chloride (30 mL), and stirred for 30 minutes, and then extracted with ethyl acetate solution (30 mL×3). The organic phase was washed with saturated saline water, dried over anhydrous sodium sulfate, and concentrated to obtain a crude product, and the crude product was purified by column chromatography (PE/EtOAc=1/1) to obtain compound 4e (155 mg).

MS(ESI) m/z 333.30 [M+H]⁺.

### Step 5 2-(5-(4-amino-1H-pyrazol-1-yl) methyl) pyridin-2-yl) propan-2-ol 4f

Compound 4f (0.47 mmol, 155 mg) was dissolved in ethanol (10 mL), and concentrated hydrochloric acid (2 mL, 38%) was added to react at room temperature for 4 hours. After the reaction was completed, it was spun dried to remove the solvent to obtain compound 4f (100 mg, crude), which can be directly used for the next reaction step.

MS(ESI) m/z 233.30 [M+H]⁺.

### Step 6 (R)—N-(1-(6-(2-hydroxypropan-2-yl) pyridin-3-yl) methyl)-1H-pyrazol-4-yl)-9-methyl-6-oxo-6,7,8,9-tetrahydropyridino [3′, 2′: 4,5]pyrrolo [1,2-a]pyrazine-2-carboxamide 4

Compound 4f (0.43 mmol, 100 mg) was dissolved in N, N-dimethylformamide (10 mL), compound 1g (0.42 mmol, 104 mg), N, N-diisopropylethylamine (0.80 mmol, 220 mg), and HATU (0.64 mmol, 242 mg) were added at room temperature, and the reaction mixture reacted at room temperature for 14 hours. After TLC monitored the completion of the reaction, it was diluted with added water (20 mL), extracted with ethyl acetate (20 mL×3), the organic phases were combined, washed with saturated saline solution, and concentrated under reduced pressure to obtain a crude product, and the crude product was purified by column chromatography (PE/EtOAc=1/1) to obtain compound 4 (44 mg), a light yellow solid.

MS(ESI) m/z 460.30 [M+H]⁺.

¹H NMR (500 MHz, CD3OD) δ 1.50 (3H, d, J=6.5 Hz), 1.53 (6H, s), 3.55 (1H, d, J=12.5 Hz), 3.98 (1H, dd, J=4.5 Hz, J=13.5 Hz), 5.30 (1H, m), 5.46 (2H, s), 7.06 (1H, d, J=5 Hz), 7.21 (1H, s), 7.57 (1H, s), 7.92 (1H, s), 7.98 (1H, d, J=5 Hz), 8.19 (1H, d, J=8.5 Hz), 8.31 (1H, s) 8.43 (1H, d, J=5 Hz).

### Example 5 (R)—N-(1-(6-(2-hydroxypropan-2-yl) pyridin-3-yl) methyl)-1H-pyrazol-4-yl)-9-methyl-6-oxo-6,7,8,9-tetrahydropyridino [3′, 2′: 4,5]pyrrolo [1,2-a]pyrazine-2-carboxamide

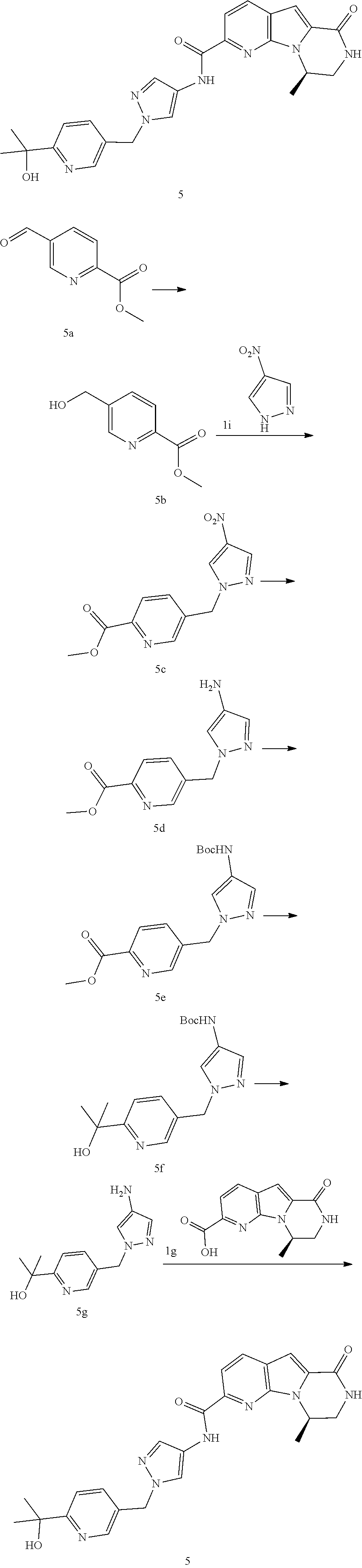

### Step 1 methyl 5-(hydroxymethyl) pyridinecarboxylate 5b

Compound 5a (27.3 mmol, 4.50 g) was dissolved in methanol (50 mL), and 1.13 g sodium borohydride (29.7 mmol, 1.13 g) was added at 0° C. to react at room temperature for 3 hours. After TLC monitored the completion of the reaction, it was diluted with added water (60 mL), extracted with ethyl acetate (60 mL×3), and the organic phases were combined, washed with saturated saline solution, and concentrated under reduced pressure to obtain crude compound 5b (4.2 g, crude), a colorless oil.

MS(ESI) m/z 168.30 [M+H]⁺.

### Step 2 methyl 5-(4-nitro-1H-pyrazol-1-yl) methyl) pyridinecarboxylate 5c

Compound 5b (4.20 g, 25.0 mmol), compound 1i (3.14 g, 27.78 mmol), and triphenylphosphine (7.30 g, 28.2 mmol) were dissolved in tetrahydrofuran (50 mL), then replaced with argon gas three times, and cooled down to 0° C., and di-tert-butyl azodicarboxylate (6.03 g, 27.33 mmol) was added, then the mixture reacted at room temperature for 24 hours. After TLC monitored the completion of the reaction, it was diluted with water (100 mL), extracted with ethyl acetate (100 mL×3), the organic phases were combined, washed with saturated saline, and concentrated under reduced pressure to obtain a crude product, and the crude product was purified by column chromatography (PE/EtOAc=2/1) to obtain compound 4b (5.7 g), a light yellow solid.

MS m/z (ESI): 234 [M+H]⁺.

¹H NMR (500 MHz, DMSO-d6) δ 3.85 (3H, s), 5.55 (2H, s), 7.86 (1H, dd, J=2 Hz, J=8 Hz), 8.03 (1H, d, J=8 Hz), 8.30 (1H, S), 8.70 (1H, d, J=2 Hz), 9.08 (1H, s).

### Step 3 methyl 5-(4-amino-1H-pyrazol-1-yl) methyl) pyridinecarboxylate 5d

Compound 5c (5.13 mmol, 1.20 g) wad dissolved in tetrahydrofuran (50 mL), and 0.3 g palladium on carbon (5%, wet basis) was added to react in H₂ (15 psi) environment for 5 hours. After TLC monitored the completion of the reaction, the reaction mixture was filtered through diatomaceous earth, the filter cake was washed with tetrahydrofuran (100 mL), and the filtrate was spun dried to obtain crude compound 5d (928 mg, crude), which was directly used for the next reaction step.

MS m/z (ESI): 233.2 [M+H]⁺.

### Step 4 methyl 5-((4-(tert-butoxycarbonyl) amino)-1H-pyrazol-1-yl) methyl) pyridinecarboxylate 5e

Compound 5d (4.00 mmol, 928 mg) was dissolved in tetrahydrofuran (50 mL), DMAP (0.196 mmol, 22.0 mg) and di-tert-butyl dicarbonate (4.50 mmol, 1.00 g) were added at 0° C., and the reaction mixture reacted at room temperature for 14 hours. After TLC monitored the completion of the reaction, it was diluted with water (50 mL), extracted with ethyl acetate (50 mL×3), the organic phases were combined, washed with saturated saline solution, and concentrated under reduced pressure to obtain a crude product, and the crude product was purified by column chromatography (PE/EtOAc=1/1) to obtain compound 5e (0.57 g).

MS(ESI) m/z 333.25 [M+H]⁺.

### Step 5 tert-butyl (1-(6-(2-hydroxypropan-2-yl) pyridin-3-yl) methyl)-1H-pyrazol-4-yl) carbamate 5f

Compound 5e (1.71 mmol, 568 mg) was dissolved in ultra dry tetrahydrofuran (20 mL), a solution of methyl magnesium bromide (6.84 mmol, 6.84 mL, 1.0 M) in tetrahydrofuran was added dropwise at −70° C., and the reaction mixture reacted at room temperature for 2 hours. After TLC monitored the completion of the reaction, it was quenched with saturated ammonium chloride (30 mL), stirred for 30 minutes, extracted with ethyl acetate (30 mL×3), the organic phase was washed with saturated saline solution, dried over anhydrous sodium sulfate, and concentrated to obtain a crude product, and the crude product was purified by column chromatography (PE/EtOAc=1/1) to obtain compound 5f (430 mg).

MS(ESI) m/z 333.30 [M+H]⁺.

### Step 6 2-(5-((4-amino-1H-pyrazol-1-yl) methyl) pyridin-2-yl) propan-2-ol 5g

Compound 4f (0.57 mmol, 190 mg) was dissolved in ethanol (10 mL), and concentrated hydrochloric acid (2 mL, 38%) was added to react at room temperature for 4 hours. After the completion of the reaction, it was spun dried to remove the solvent to obtain compound 5g (150 mg, crude), which was directly used for the next reaction step.

MS(ESI) m/z 233.30 [M+H]⁺.

### Step 7 (R)—N-(1-(6-(2-hydroxypropan-2-yl) pyridin-3-yl) methyl)-1H-pyrazol-4-yl)-9-methyl-6-oxo-6,7,8,9-tetrahydropyridino [3′, 2′: 4,5]pyrrolo [1,2-a]pyrazine-2-carboxamide 5

Compound 5g (0.53 mmol, 133 mg) was dissolved in N, N-dimethylformamide (10 mL), and compound 1g (0.55 mmol, 128 mg), N, N-diisopropylethylamine (1.10 mmol, 270 mg), and HATU (0.71 mmol, 297 mg) were added at room temperature, and the reaction mixture reacted at room temperature for 14 hours. After TLC monitored the completion of the reaction, it was diluted with water (20 mL) and extracted with ethyl acetate (20 mL×3), the organic phases were combined, washed with saturated saline, and concentrated under reduced pressure to obtain a crude product, and the crude product was purified by column chromatography (PE/EtOAc=1/1) to obtain compound 5 (59 mg), a light yellow solid.

MS(ESI) m/z 460.30 [M+H]⁺.

¹H NMR (500 MHz, CDCl₃) δ 1.53 (6H, s), 1.59 (3H, d, J=6.5 Hz), 3.57 (1H, dd, J=5 Hz, J=12.5 Hz), 4.07 (1H, dd, J=4.5 Hz, J=12.5 Hz), 5.23 (1H, m), 5.35 (2H, s), 6.82 (1H, s), 7.29 (1H, s), 7.37 (1H, d, J=8 Hz), 7.62 (1H, d, J=7.5 Hz), 7.69 (1H, s), 8.13 (1H, d, J=8 Hz), 8.20 (1H, d, J=8.5 Hz), 8.26 (1H, s), 8.48 (1H, s), 9.69 (1H, s).

### Example 6 (R)-1-(4-aminobenzyl)-N-(3-fluoro-9-methyl-6-oxo-6,7,8,9-tetrahydropyridino [3′, 2″: 4,5]pyrrolo [1,2-a]pyrazin-2-yl)-1H-pyrazole-4-carboxamide

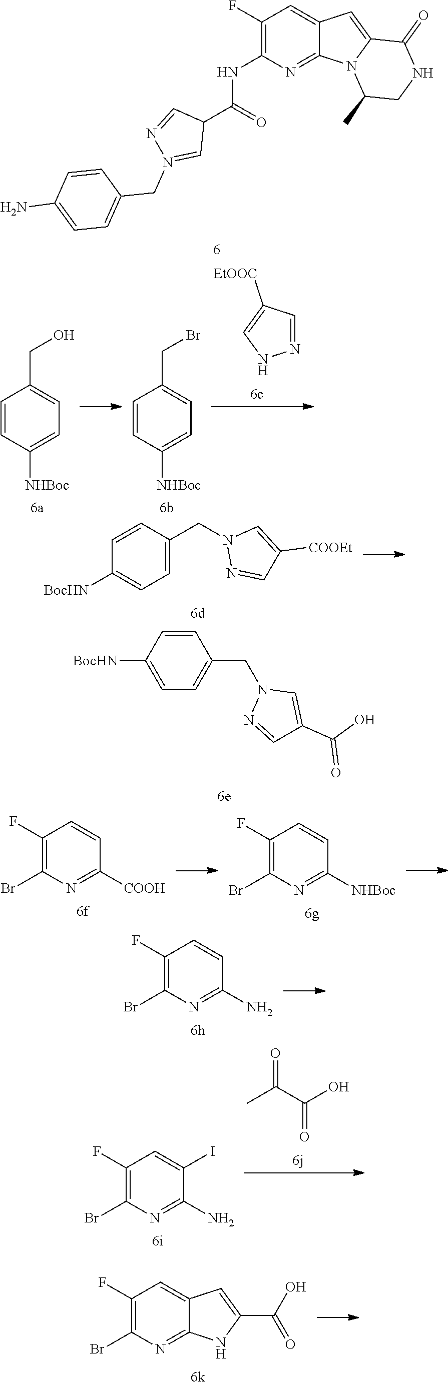

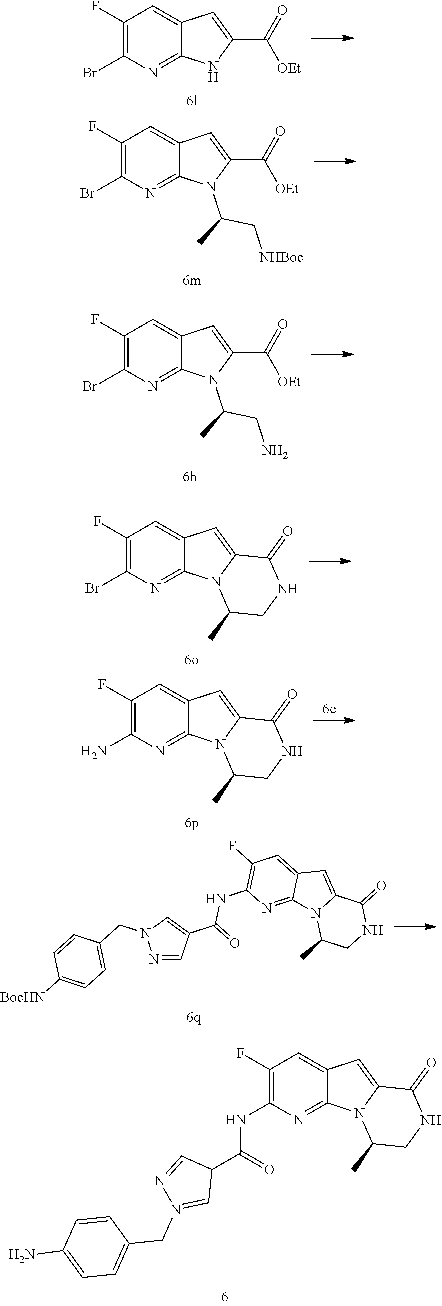

### Step 1 tert-butyl (4-(bromomethyl) phenyl) carbamate 6b

Compound 6a (1.12 g, 5 mmol) was dissolved in anhydrous ether (50 mL), and phosphorus tribromide (0.16 mL, 1.75 mmol) was added dropwise at −10° C. to react at 0° C. for 1 hour. After TLC monitored the completion of the reaction, it was diluted with saturated sodium bicarbonate (50 mL), extracted with ethyl acetate (50 mL×3), the organic phases were combined, washed with saturated saline solution, and concentrated under reduced pressure to obtain crude compound 6b (1.4 g, crude), a white solid.

MS(ESI) m/z 286.20 [M+H]⁺.

1H-NMR (400 MHz, CDCl₃) δ 7.34-7.30 (m, 4H), 6.53 (s, 1H), 4.48 (s, 2H), 1.51 (s, 9H).

### Step 2 ethyl 1-(4-(tert butoxycarbonyl) amino) benzyl)-1H-pyrazole-4-carboxylate 6d

Compound 6b (315 mg, 1.10 mmol) and compound 6c (140 mg, 1.00 mmol) were dissolved in anhydrous acetone (10 mL), anhydrous potassium carbonate (276 mg, 2.00 mmol) was added, and the reaction mixture was heated to reflux for 18 hours. After TLC monitored the completion of the reaction, it was filtrated to remove the solid, the filtrate was concentrated under reduced pressure to obtain a crude compound, and the crude compound was purified by column chromatography (PE/EtOAc=3:1) to obtain 6d (310 mg).

MS (ESI): m/z 368.10 [M+Na]⁺.

1H-NMR (500 MHz, CDCl₃) δ 7.90 (s, 1H), 7.79 (s, 1H), 7.36 (d, J=8.3 Hz, 2H), 7.16 (d, J=8.6 Hz, 2H), 6.80 (s, 1H), 5.21 (s, 2H), 4.24 (q, J=7.1 Hz, 2H), 1.48 (s, 9H), 1.29 (t, J=7.1 Hz, 3H).

### Step 3 1-(4-(tert butoxycarbonyl) amino) benzyl)-1H-pyrazole-4-carboxylic acid 6e

Compound 6d (310 mg, 0.9 mmol) was dissolved in methanol (5 mL), 2.0 M sodium hydroxide solution (2.25 mL, 4.5 mmol) was added, and the reaction solution was heated up to 65° C. for 4 hours. After TLC monitored the completion of the reaction, the reaction mixture was cooled down to 0° C., and the pH was adjusted to 4-5 by adding ice acetic acid dropwise. The aqueous phase was extracted with dichloromethane (50 mL×3), the organic phases were combined, washed with saturated saline solution, and concentrated under reduced pressure to obtain crude compound 6e (258 mg, crude), which was directly used for the next reaction step.

MS (ESI): m/z 340 [M+Na]+.

1H-NMR (500 MHz, CDCl₃) δ 7.98 (s, 1H), 7.85 (s, 1H), 7.35 (d, J=8.2 Hz, 2H), 7.19 (d, J=8.1 Hz, 2H), 6.78 (s, 1H), 5.24 (s, 2H), 1.50 (s, 9H).

### Step 4 tert-butyl (6-bromo-5-fluoropyridin-2-yl) carbamate 6g

Compound 6f (11.0 g, 50.0 mmol) was dissolved in anhydrous tert-butanol (200 mL), and triethylamine (6.90 mL, 50.0 mmol) and diphenyl azide phosphate (11.0 mL, 50.0 mmol) were added dropwise at room temperature, and the reaction solution was heated up to 85° C. for 3 hours. After TLC monitored the completion of the reaction, the reaction mixture was evaporated by rotary evaporation to remove the solvent, diluted with added water (100 mL), extracted with ethyl acetate (100 mL×3), the organic phases were combined, washed with saturated saline, and concentrated under reduced pressure to obtain a crude compound, and the crude compound was purified by column chromatography (PE/EtOAc=20:1) to obtain 6g (12 g), a light yellow oil.

MS (ESI): m/z 313.15 [M+Na]+.

1H-NMR (400 MHz, CDCl₃) δ 7.89 (dd, J=8.9, 3.2 Hz, 1H), 7.40 (dd, J=8.9, 6.9 Hz, 1H), 7.25 (brs, 1H), 1.50 (s, 9H).

### Step 5 6-Bromo-5-fluoropyridine-2-amine 6h

Compound 6g (12.0 g, 41.0 mmol) was dissolved in anhydrous dichloromethane (100 mL), and trifluoroacetic acid (31.0 mL, 410 mmol) was added dropwise at room temperature to react for 3 hours at room temperature. After TLC monitored the completion of the reaction, the reaction mixture was evaporated by rotary evaporation to remove the solvent, diluted with saturated sodium bicarbonate (100 mL), extracted with ethyl acetate (100 mL×3), the organic phases were combined, dried over anhydrous sodium sulfate, and then concentrated under reduced pressure to obtain crude compound 6h (7.7 g, crude), which was directly used for the next reaction step.

MS (ESI): m/z 191.10 [M+H]⁺.

1H-NMR (500 MHz, CDCl₃) δ 7.16 (dd, J=8.7, 7.2 Hz, 1H), 6.36 (dd, J=8.7, 2.7 Hz, 1H), 4.75 (s, 2H).

### Step 6 6-bromo-5-fluoro-3-iodopyridine-2-amine 6i

Compound 6h (7.70 g, 40.0 mmol) was suspended in acetic acid (70 mL), NIS (9.90 g, 44.0 mmol) was added at room temperature, and trifluoroacetic acid (0.70 mL) was added dropwise, and finally the reaction mixture reacted at room temperature for 3 hours. After TLC monitored the completion of the reaction, saturated ammonia water was added dropwise at 0° C. to adjust the pH to 9. The mixture was extracted with ethyl acetate (100 mL×3), the organic phases were combined, dried over anhydrous sodium sulfate and then concentrated under reduced pressure to obtain a crude compound, and the crude compound was purified by column chromatography (PE/EtOAc=10:1) to obtain 6i (10.0 g), a red solid.

MS (ESI): m/z 317.20 [M+H]⁺.

1H-NMR (500 MHz, CDCl₃) δ 7.62 (d, J=6.4 Hz, 1H), 4.94 (s, 2H).

### Step 7 6-bromo-5-fluoro-1H-pyrrolo [2,3-b]pyridine-2-carboxylic acid 6k

Compound 6i (5.10 g, 16.0 mmol), DABCO (5.40 g, 48.0 mmol), and palladium acetate (0.36 g, 1.60 mmol) was added into a sealed tube, and replaced with argon three times, and then ultra dry DMF (50 mL) and pyruvic acid (3.30 mL, 48.0 mmol) were added to react at 110° C. for 3 hours. After TLC monitored the completion of the reaction, the reaction mixture was filtered to remove the solid, and the solid residue was washed with a small amount of DMF, evaporated by rotary evaporation to remove the solvent, and pumped dry by oil pump to obtain 6k (4.50 g, crude), black brown oil, which was directly used for the next reaction step without purification.

MS (ESI): m/z 259.10 [M+H]⁺.

### Step 8 ethyl 6-bromo-5-fluoro-1H-pyrrolo [2,3-b]pyridine-2-carboxylate 6l

Compound 6k (4.50 g, crude) was dissolved in anhydrous ethanol (300 mL), and concentrated sulfuric acid (9 mL) was added dropwise at room temperature, and the reaction was heated to reflux for 18 hours. After TLC monitored the completion of the reaction, the reaction mixture was cooled down to 0° C. and saturated sodium bicarbonate (70 mL) was added dropwise, and then it was evaporated by rotary evaporation to remove the solvent and extracted with ethyl acetate (100 mL×3). The organic phases were combined, dried over anhydrous sodium sulfate, and concentrated under reduced pressure to obtain a crude compound, and the crude compound was purified by column chromatography (PE/EtOAc/Et3N=6:1:0.05) to obtain 6l (1.10 g), white solid.

MS (ESI): m/z 287.10 [M+H]+.

1H-NMR (400 MHz, CDCl₃) δ 9.20 (s, 1H), 7.73 (d, J=7.8 Hz, 1H), 7.14 (d, J=1.9 Hz, 1H), 4.43 (q, J=7.2 Hz, 2H), 1.42 (t, J=7.2 Hz, 3H).

### Step 9 ethyl (R)-6-bromo-1-(1-(tert-butoxycarbonyl) amino) propan-2-yl)-5-fluoro-1H-pyrrolo [2,3-b]pyridine-2-carboxylate 6m

Sodium hydride (96.0 mg, 2.40 mmol, 60% in mineral oil) was added to a reaction flask, and replaced with argon gas, and then ultra dry DMF (3 mL) was added, and a DMF (10 mL) solution of compound 6l (574 mg, 2.00 mmol) was added dropwise at 0° C. to react at 0° C. for half an hour, then a DMF (6 mL) solution of tert-butyl (S)-5-methyl-1-oxa-2,2-dioxo-2,3-thiazolidine-3-carbonate (498 mg, 2.10 mmol) was added dropwise. After the completion of the addition, the reaction was carried out at room temperature for 18 hours. After TLC monitored the completion of the reaction, it was diluted with added water (50 mL), extracted with ethyl acetate (50 mL×3), the organic phases were combined, dried over anhydrous sodium sulfate, and then concentrated under reduced pressure to obtain a crude compound, and the crude compound was purified by column chromatography (PE/EtOAc/Et3N=6:1:0.05) to obtain a white solid 6m (0.70 g).

MS (ESI): m/z 443.10 [M+H]+.

1H-NMR (500 MHz, CDCl₃) δ 7.65 (d, J=7.8 Hz, 1H), 7.18 (s, 1H), 5.75 (m, 1H), 4.85 (s, 1H), 4.38 (q, J=7.0 Hz, 2H), 4.07-4.00 (m, 1H), 3.63-3.58 (m, 1H), 1.67 (d, J=7.1 Hz, 3H), 1.40 (t, J=7.0 Hz, 3H), 1.34 (s, 9H).

### Step 10 ethyl (R)-1-(1-aminopropan-2-yl)-6-bromo-5-fluoro-1H-pyrrolo [2,3-b]pyridine-2-carboxylate 6n

Compound 6m (0.70 g, 1.60 mmol) was dissolved in anhydrous dichloromethane (8 mL), and trifluoroacetic acid (3 mL) was added dropwise at room temperature to react for 3 hours at room temperature. After TLC monitored the completion of the reaction, the reaction mixture was evaporated by rotary evaporation to remove the solvent, and pumped dry by oil pump to obtain a brownish yellow foam like solid 6n (0.75 g, crude product), which was directly used for the next reaction step without purification.

MS (ESI): m/z 344.10 [M+H]+.

### Step 11 (R)-2-bromo-3-fluoro-9-methyl-8,9-dihydropyridino [3′, 2′: 4,5]pyrrolo [1,2-a]pyrazine-6 (7H)-one 6o

Compound 6n (0.75 g, crude) was dissolved in anhydrous methanol (20 mL), and anhydrous potassium carbonate (1.38 g, 10.0 mmol) was added at room temperature to react at room temperature for 18 hours. After TLC monitored the completion of the reaction, the reaction mixture was evaporated by rotary evaporation to remove the solvent, diluted with added water (30 mL), and extracted with ethyl acetate (30 mL×3). The organic phases were combined, dried over anhydrous sodium sulfate, and concentrated under reduced pressure to obtain crude compound 6o (0.60 g, crude), which was directly used for the next reaction step.

MS (ESI): m/z 320, 322 [M+Na]+.

¹H-NMR (500 MHz, CDCl₃) δ 7.73 (d, J=7.9 Hz, 1H), 7.34 (m, 1H), 7.17 (s, 1H), 5.09-5.04 (m, 1H), 4.01 (dd, J=12.8, 4.5 Hz, 1H), 3.53 (ddd, J=12.8, 5.1, 1.6 Hz, 1H), 1.52 (d, J=6.6 Hz, 3H).

Step 12 (R)-2-amino-3-fluoro-9-methyl-8,9-dihydropyridino [3′, 2′: 4,5]pyrrolo [1,2-a]pyrazine-6 (7H)-one 6p

Compound 6o (298 mg, 1.00 mmol), cuprous iodide (19.0 mg, 0.10 mmol), ligand N¹, N²-bis(5-methyl-[1,1′-biphenyl]-2-yl) oxalyl diamine (BPPPO) (42.0 mg, 0.10 mmol), and anhydrous potassium phosphate (318 mg, 1.50 mmol) were added into the sealed tube, and replaced with argon gas three times, then ultra dry DMSO (3 mL) and ammonia water (25%, 0.2 mL, 3 mmol) were added, and the sealed tube was heated up to 80° C. to react for 18 hours. After TLC monitored the completion of the reaction, it was diluted with added water (30 mL), extracted with ethyl acetate (30 mL×3), the organic phases were combined, dried over anhydrous sodium sulfate, and then concentrated under reduced pressure to obtain crude compound 6p (0.18 g, crude), which was directly used for the next reaction step.

MS (ESI): m/z 235.20 [M+H]⁺.

¹H-NMR (500 MHz, CDCl₃) δ 7.50 (d, J=10.8 Hz, 1H), 7.08 (s, 1H), 6.26 (s, 1H), 4.89-4.84 (m, 1H), 4.77 (s, 2H), 3.96 (dd, J=12.6, 4.4 Hz, 1H), 3.43 (ddd, J=12.5, 5.1, 1.6 Hz, 1H), 1.46 (d, J=6.6 Hz, 3H).

Step 13 tert-butyl (R)-(4-((3-fluoro-9-methyl-6-oxo-6,7,8,9-tetrahydropyridino [3′, 2]: 4,5]pyrrolo [1,2-a]pyrazin-2-yl) carbamoyl)-1H-pyrazol-1-yl) methyl) phenylcarbamate 6q

Compound 6p (24.0 mg, 0.10 mmol), compound 6e (32.0 mg, 0.10 mmol), and PyCIU (50.0 mg, 0.15 mmol) were added into the sealed tube, and ultra dry dichloroethane (5 mL) and DIPEA (0.07 mL, 0.4 mmol) were added, and the sealed tube was heated up to 80° C. to react for 18 hours. After TLC monitored the completion of the reaction, it was diluted with water (30 mL), extracted with ethyl acetate (30 mL×3), and the organic phases were combined, dried over anhydrous sodium sulfate, and then concentrated under reduced pressure to obtain a crude compound, and the crude compound was purified by column chromatography (PE/EtOAc/Et3N=2:1:0.05) to obtain 6q (10.0 mg), a light yellow solid.

MS (ESI): m/z 556 [M+Na]⁺.

1H-NMR (500 MHz, CDCl₃) δ 7.92 (s, 1H), 7.92 (s, 1H), 7.50 (d, J=10.6 Hz, 1H), 7.35 (d, J=8.1 Hz, 2H), 7.23 (s, 1H), 7.21 (d, J=8.3 Hz, 2H), 6.50 (s, 1H), 5.26 (d, J=15.0 Hz, 1H), 5.22 (d, J=15.0 Hz, 1H), 4.88 (s, 2H), 4.43 (dd, J=13.5, 2.0 Hz, 1H), 4.00-3.95 (m, 1H), 1.51 (s, 1H) 9H), 1.50 (d, J=6.6 Hz, 3H).

### Step 14 (R)-1-(4-aminobenzyl)-N-(3-fluoro-9-methyl-6-oxo-6,7,8,9-tetrahydropyridino [3′, 2″: 4,5]pyrrolo [1,2-a]pyrazin-2-yl)-1H-pyrazole-4-carboxamide 6

Compound 6q (10.0 mg, 0.019 mmol) was dissolved in anhydrous dichloromethane (2 mL), and trifluoroacetic acid (0.50 mL) was added dropwise at room temperature to react for 4 hours at room temperature. After TLC monitored the completion of the reaction, it was evaporated by rotary evaporation to remove the solvent, diluted with added saturated sodium bicarbonate (30 mL), extracted with dichloromethane (30 mL×3), the organic phases were combined, dried over anhydrous sodium sulfate, and then concentrated under reduced pressure to obtain a crude compound, and the crude compound was purified by column chromatography (DCM/MeOH/Et3N=100:5:1) to obtain a light yellow solid 6 (3.7 mg).

MS (ESI): m/z 434 [M+H]+.

¹H NMR (500 MHz, CD3OD) δ 8.25 (s, 1H), 7.87 (s, 1H), 7.57 (d, J=11.1 Hz, 1H), 7.30 (d, J=8.4 Hz, 2H), 7.21 (s, 1H), 7.09 (d, J=8.4 Hz, 2H), 5.32 (s, 2H), 4.94 (m, 1H), 4.39 (dd, J=13.5, 2.0 Hz, 1H), 4.00 (dd, J=13.5, 4.0 Hz, 1H), 1.50 (d, J=6.6 Hz, 3H).

### Example 7 (R)—N-(1-(4-aminobenzyl)-1H-pyrazol-4-yl)-6-fluoro-4-methyl-1-oxo-1,2,3,4-tetrahydropyrazino [1,2-a]indole-7-carboxamide

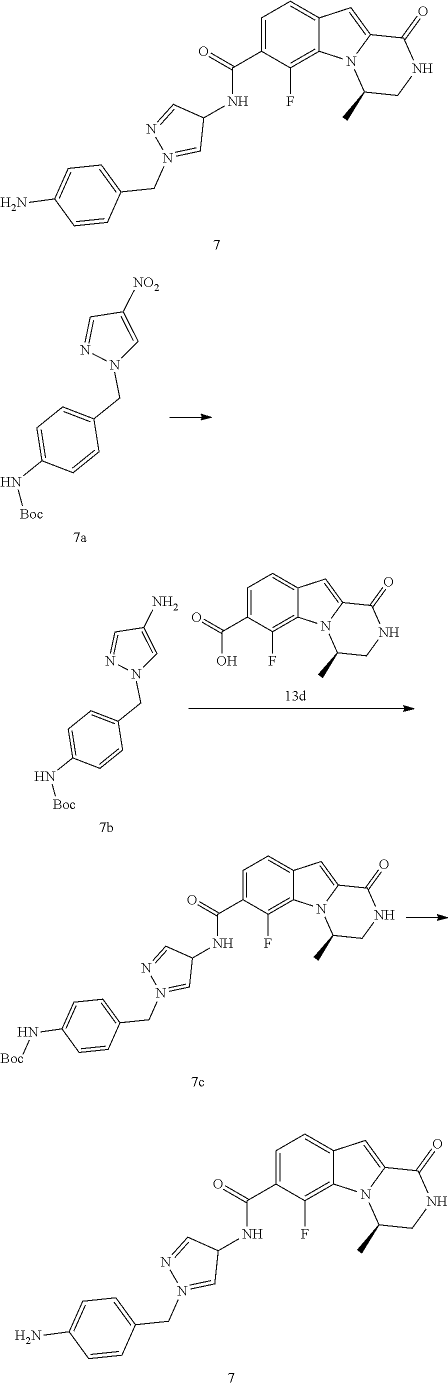

### Step 1 tert-butyl (4-(4-amino-1H-pyrazol-1-yl) methyl) phenyl) carbamate 7b

To a methanol (10 mL) solution of compound 7a (105 mg, 0.33 mmol), palladium carbon (16 mg) was added to react for 5 hours under hydrogen gas (15 psi) environment. After TLC monitored the completion of the reaction, the reaction mixture was filtered through diatomaceous earth, the filter cake was washed with tetrahydrofuran (10 mL), and the filtrate was spun dried to obtain crude compound 7b (90 mg), an orange solid.

¹H NMR (400 MHz, DMSO-d6) δ 9.36 (s, 1H), 7.38 (d, J=8.4 Hz, 2H), 7.11-7.07 (m, 2H), 7.01 (d, J=0.9 Hz, 1H), 6.91 (d, J=0.9 Hz, 1H), 5.02 (s, 2H), 1.46 (s, 9H).

### Step 2 tert-butyl (R)-(4-((4-(6-fluoro-4-methyl-1-oxo-1,2,3,4-tetrahydropyrazino [1,2-a]indole-7-carboxamide)-1H-pyrazol-1-yl) methyl) phenyl) carbamate 7c

Under argon gas protection, compounds 13d (80 mg, 0.30 mmol), 7b (86 mg, 0.30 mmol), HATU (170 mg, 0.45 mmol), DIPEA (116 mg, 0.89 mmol), and DMF (5 mL) were sequentially added to a 50 mL three necked flask, and the reaction was carried out at room temperature for 24 hours. After TLC monitored the completion of the reaction, the reaction mixture was diluted with water and extracted with ethyl acetate (50 mL×3). The organic phases were combined, washed with saturated saline solution, and concentrated under reduced pressure to obtain a crude product, and the crude product was purified by column chromatography to obtain compound 7c (25 mg), a white solid.

¹H NMR (400 MHz, CD3OD) δ 7.98 (d, J=0.7 Hz, 1H), 7.62 (d, J=0.7 Hz, 1H), 7.46 (d, J=8.4 Hz, 1H), 7.30 (dt, J=8.5, 3.0 Hz, 3H), 7.15 (d, J=2.2 Hz, 1H), 7.12-7.08 (m, 2H), 5.17 (s, 2H), 5.04 (q, J=6.8, 6.2 Hz, 1H), 3.93 (dd, J=13.3, 4.4 Hz, 1H), 3.45 (dd, J=13.3, 1.3 Hz, 1H), 1.41 (d, J=2.1 Hz, 12H).

### Step 3 (R)—N-(1-(4-aminobenzyl)-1H-pyrazol-4-yl)-6-fluoro-4-methyl-1-oxo-1,2,3,4-tetrahydropyrazino [1,2-a]indole-7-carboxamide 7

Compound 7c (25 mg) was placed in a 25 mL round bottom flask, a 1,4-dioxane solution of hydrochloric acid (2 mL, 4.0M) was added dropwise slowly, and the reaction was carried out at room temperature for 2 hours. After the reaction was completed, it was concentrated under reduced pressure, neutralized with added saturated sodium bicarbonate solution, extracted with dichloromethane, the organic phases were combined, washed with saturated saline water, and concentrated under reduced pressure to obtain a crude product, and the crude product was purified by column chromatography to obtain compound 7 (11 mg), a yellow solid.

MS (ESI) m/z 433 [M+H]⁺.

¹H NMR (400 MHz, CD3OD) δ 8.08 (s, 1H), 7.62 (s, 1H), 7.47 (d, J=8.4 Hz, 1H), 7.36-7.19 (m, 5H), 7.16 (s, 1H), 5.29 (s, 2H), 5.06-5.03 (m, 1H), 3.92 (t, J=5.4 Hz, 1H), 3.45 (d, J=13.4 Hz, 1H), 1.41 (d, J=6.6 Hz, 3H).

### Example 8 (9R)—N-(1-(1-(6-(2-hydroxyprop-2-yl) pyridin-3-yl) ethyl)-1H-pyrazol-4-yl)-9-methyl-6-oxo-6,7,8,9-tetrahydropyridino [3′, 2′: 4,5]pyrrolo [1,2-a]pyrazine-2-carboxamide

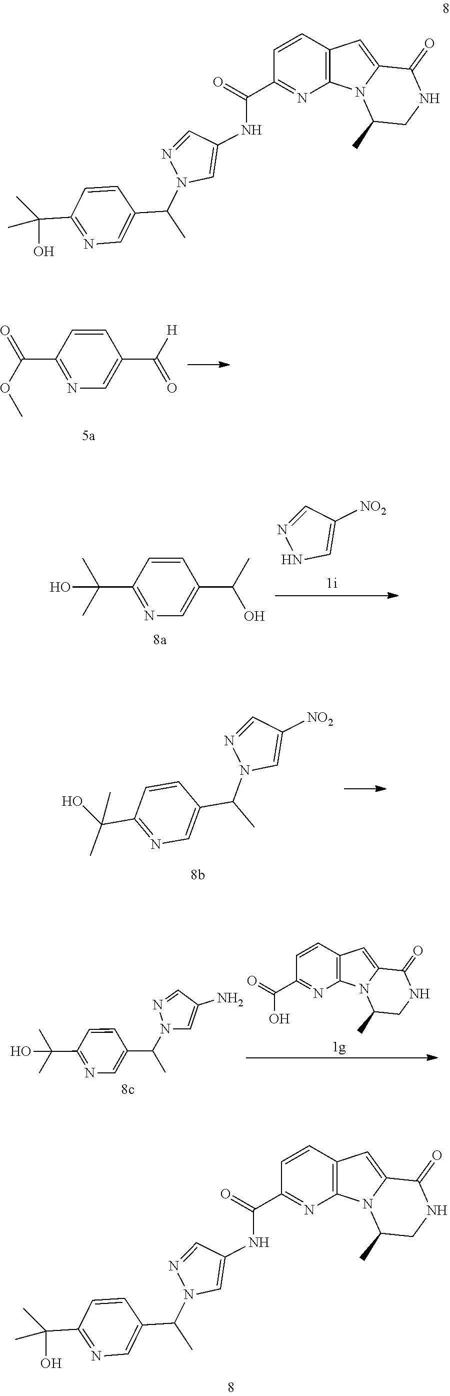

### Step 1 2-(5-(1-hydroxyethyl) pyridin-2-yl) propan-2-ol 8a

Compound 5a (1 g, 6 mmol) was dissolved in THF (10 mL), and methyl magnesium bromide (36 mL, 36 mmol, LOM) was added dropwise in an ice bath, and then the reaction was carried out at room temperature for 3 hours. After the reaction was completed, it was quenched with added saturated ammonium chloride solution (10 mL), extracted with dichloromethane (25 mL), the organic phase was washed with saturated saline solution, and concentrated under reduced pressure to obtain a crude product, and the crude product was purified by column chromatography (PE/EtOAc=10/1) to obtain compound 8a (0.57 g).

MS (ESI) m/z 182 [M+H]⁺

¹H NMR (400 MHz, CDCl₃) δ 8.49 (s, 1H), 7.76 (d, J=8.33, 1H), 7.36 (d, J=8.33 Hz, 1H), 4.97 (m, 1H), 1.47-1.58 (m, 9H).

### Step 2 2-(5-(1-(4-nitro-1H-pyrazol-1-yl) ethyl) pyridin-2-yl) propan-2-ol 8b

Compound 8a (0.57 g, 3.15 mmol) was dissolved in anhydrous tetrahydrofuran (20 mL), and 4-nitropyrazole (0.39 g, 3.45 mmol) and triphenylphosphine (2.48 g, 9.45 mmol) were added. The reaction was placed in an ice bath, and DBAD (1.45 g, 6.3 mmol) was added in batches to react at room temperature for 16 hours. After the reaction was completed, saturated sodium bicarbonate solution (10 mL) was added, and it was extracted with ethyl acetate (20 mL×3). The organic phases were combined, washed with saturated saline solution (10 mL), and concentrated under reduced pressure to obtain a crude product, and the crude product was purified by column chromatography (PE/EtOAc=5/1) to obtain compound 8b (200 mg).

MS (ESI) m/z 277 [M+H]⁺.

¹H NMR (400 MHz, CDCl₃) δ 8.49 (m, 1H), 8.22 (d, 1H), 8.10 (d, 1H), 7.66 (m, 1H), 7.42 (m, 1H), 5.58 (m, 1H), 1.96 (m, 3H), 53 (m, 6H).

### Step 3 2-(5-(1-(4-amino-1H-pyrazol-1-yl) ethyl) pyridin-2-yl) propan-2-ol 8c

Compound 8b (200 mg, 0.54 mmol) was dissolved in methanol (5 mL), and then Pd/C and ammonium formate (1 g) were added under nitrogen gas to react at room temperature for 3 hours. After the reaction was completed, it was filtered, and then concentrated under reduced pressure to obtain crude product 8c (200 mg), which was directly used for the next reaction step.

MS (ESI) m/z 247 [M+H]⁺.

### Step 4 (9R)—N-(1-(1-(6-(2-hydroxyprop-2-yl) pyridin-3-yl) ethyl)-1H-pyrazol-4-yl)-9-methyl-6-oxo-6,7,8,9-tetrahydropyridino [3′, 2′: 4,5]pyrrolo [1,2-a]pyrazine-2-carboxamide 8

Compound 8c (40 mg, 0.16 mmol) was dissolved in DMF (2 mL), and then compound 1g (40 mg, 0.16 mmol), HATU (60 mg, 0.19 mmol), and DIPEA (62 mg, 0.48 mmol) were added to react at room temperature for 3 hours. After the reaction was completed, water (10 mL) was added, and extracted with ethyl acetate (20 mL×3), the organic phases were combined, washed with saturated saline water (50 mL), concentrated under reduced pressure to obtain a crude product, and the crude product was purified by column chromatography (DCM/MeOH=10/1) to obtain compound 8 (52 mg).

MS (ESI) m/z 474 [M+H]⁺.

¹H NMR (400 MHz, CDCl₃) δ 10.61 (s, 1H), 8.44 (d, 1H), 8.33-8.28 (m, 2H), 8.26 (s, 1H), 7.92 (d, 1H), 7.83 (s, 1H), 7.66 (m, 2H), 7.14 (s, 1H), 5.74-5.65 (m, 1H), 5.38-5.22 (m, 1H), 5.16 (s, 1H), 3.92 (dd, 1H), 3.48 (dd, 1H), 1.85 (m, 3H), 1.42 (m, 9H).

### Example 9 (R)—N-(1-(6-(2-hydroxypropan-2-yl) pyridin-3-yl) methyl)-5-methyl-1H-pyrazol-3-yl)-9-methyl-6-oxo-6,7,8,9-tetrahydropyridino [3′, 2′:4, 5]pyrrolo [1,2-a]pyrazin-2-carboxamide

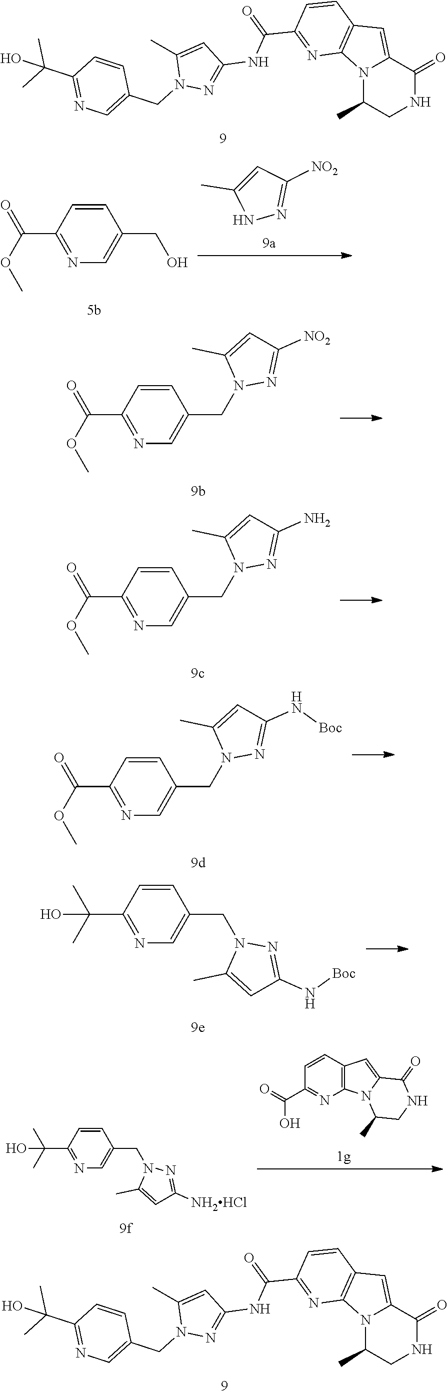

### Step 1 methyl 5-((5-methyl-3-nitro-1H-pyrazol-1-yl) methyl) pyridinecarboxylate 9b

Compound 5b (1.2 g, 7.2 mmol) was dissolved in anhydrous tetrahydrofuran (20 mL), and compound 9a (1.4 g, 10.8 mmol) and triphenylphosphine (5.66 g, 21.6 mmol) were added. The reaction was placed in an ice bath, and DBAD (3.3 g, 14.4 mmol) was added in batches to react at room temperature for 16 hours. After the reaction was completed, saturated sodium bicarbonate solution (250 mL) was added, and extracted with ethyl acetate (50 mL×3). The organic phases were combined, washed with saturated saline solution (10 mL), and concentrated under reduced pressure to obtain a crude product, and the crude product was purified by column chromatography (PE/EtOAc=6/1) to obtain compound 9b (560 mg).

MS (ESI) m/z 277 [M+H]⁺.

¹H NMR (400 MHz, CDCl₃) δ 8.67 (d, 1H), 8.15 (d, 1H), 7.68 (d, 1H), 6.75 (s, 1H), 5.46 (s, 1H), 4.03 (s, 3H), 2.31 (s, 3H).

### Step 2 methyl 5-((3-amino-5-methyl-1H-pyrazol-1-yl) methyl) pyridinecarboxylate 9c

Compound 9b (560 mg, 2.0 mmol) was dissolved in methanol (25 mL), Pd/C and ammonium formate (1.5 g) was added under nitrogen gas, and the reaction was carried out at room temperature for 3 hours. After the reaction was completed, it was filtered and concentrated under reduced pressure to obtain a crude product, and the crude product was purified by column chromatography (PE/EtOAc=2/1) to obtain compound 9c (600 mg), which was used for the next reaction step.

MS (ESI) m/z 247 [M+H]⁺.

¹H NMR (400 MHz, CDCl₃) δ 8.60 (d, 1H), 8.08 (d, 1H), 7.69 (d, 1H), 5.46 (s, 1H), 5.24 (s, 2H), 4.01 (s, 3H), 3.42 (s, 2H), 2.19 (s, 3H).

### Step 3 methyl 5-((3-(tert butoxycarbonyl) amino)-5-methyl-1H-pyrazol-1-yl) methyl) pyridinecarboxylate 9d

Compound 9c (600 mg, 2.4 mmol) was dissolved in DCM (20 mL), and triethylamine (480 mg, 4.8 mmol) and di tert-butyl dicarbonate (780 mg, 3.6 mmol) were added to react at room temperature for 3 hours. After the reaction was completed, water (20 mL) was added, and extracted with ethyl acetate (40 mL×3), the organic phases were combined, washed with saturated saline water (50 mL), and concentrated under reduced pressure to obtain a crude product, and the crude product was purified by column chromatography (DCM/MeOH=10/1) to obtain compound 9d (0.5 g).

MS (ESI) m/z 347 [M+H]⁺.

¹H NMR (400 MHz, CDCl₃) δ 8.67 (s, 1H), 8.10 (d, 1H), 7.71 (d, 1H), 6.00 (s, 1H), 5.17 (s, 2H), 4.02 (s, 3H), 2.29 (s, 3H), 1.33 (s, 9H).

### Step 4 tert-butyl (1-(6-(2-hydroxypropan-2-yl) pyridin-3-yl) methyl)-5-methyl-1H-pyrazol-3-yl) aminoformate 9e

Compound 9d (0.45 g, 1.3 mmol) was dissolved in anhydrous THF (15 mL), MeMgBr (5.8 mL, 5.8 mmol) was added dropwise under an ice bath, and the reaction was carried out at room temperature for 3 hours. After the reaction was completed, it was quenched with added saturated ammonium chloride solution (10 mL), extracted with dichloromethane (25 mL), and washed with saturated saline solution (10 mL). The organic phase was concentrated under reduced pressure to obtain a crude product, and the crude product was purified by column chromatography (PE/EtOAc=10/1) to obtain compound 9e (0.17 g).

MS (ESI) m/z 347 [M+H]⁺.

¹H NMR (400 MHz, CDCl₃) δ 8.29 (s, 1H), 7.46 (d, 1H), 7.25 (d, 1H), 6.05 (s, 1H), 5.92 (s, 1H), 5.12 (s, 2H), 2.16 (s, 3H), 1.45 (s, 6H), 1.39 (s, 9H).

### Step 5 2-(5-(3-amino-5-methyl-1H-pyrazol-1-yl) methyl) pyridin-2-yl) propan-2-ol hydrochloride 9f

Compound 9e (0.15 g mg, 0.43 mmol) was dissolved in methanol (5 mL), and a 1,4-dioxane solution of hydrochloric acid (5 mL, 4.0 M) was added to react at room temperature for 3 hours. After the reaction was completed, it was concentrated under reduced pressure to obtain crude compound 9f (150 mg), which was directly used for the next reaction step.

MS (ESI) m/z 247 [M+H]⁺.

### Step 6 (R)—N-(1-(6-(2-hydroxypropan-2-yl) pyridin-3-yl) methyl)-5-methyl-1H-pyrazol-3-yl)-9-methyl-6-oxo-6,7,8,9-tetrahydropyridino [3′, 2′: 4,5]pyrrolo [1,2-a]pyrazine-2-carboxamide 9

Compound 9f (50 mg, 0.2 mmol) was dissolved in DMF (2 mL), and then compound 1g (59 mg, 0.24 mmol), HATU (91 mg, 0.24 mmol), and DIPEA (92 mg, 0.72 mmol) were added to react at room temperature for 3 hours. After TLC monitored the completion of the reaction, ethyl acetate (20 mL) and water (10 mL) were added, and extracted with ethyl acetate (20 mL×3), the organic phases were combined, washed with saturated saline water (50 mL), concentrated under reduced pressure to obtain a crude product, and the crude product was purified by column chromatography (DCM/MeOH=10/1) to obtain compound 9 (38 mg).

MS (ESI) m/z 474 [M+H]⁺.

¹H NMR (400 MHz, CDCl₃) δ 8.34 (s, 1H), 8.15-8.10 (m, 2H), 7.45 (m, 1H), 7.30 (m, 1H), 7.22 (s, 1H), 6.77 (s, 1H), 5.66 (m, 1H), 5.20-5.13 (m, 4H), 4.01 (dd, 1H), 3.46 (m, 2H), 2.26 (s, 3H), 1.56 (m, 3H), 0.81 (s, 6H).

### Example 10 (R)—N-(1-(6-(1-hydroxycyclopropyl) pyridin-3-yl) methyl)-1H-pyrazol-4-yl)-9-methyl-6-oxo-6,7,8,9-tetrahydropyridino [3′, 2′: 4,5]pyrrolo [1,2-a]pyrazine-2-carboxamide

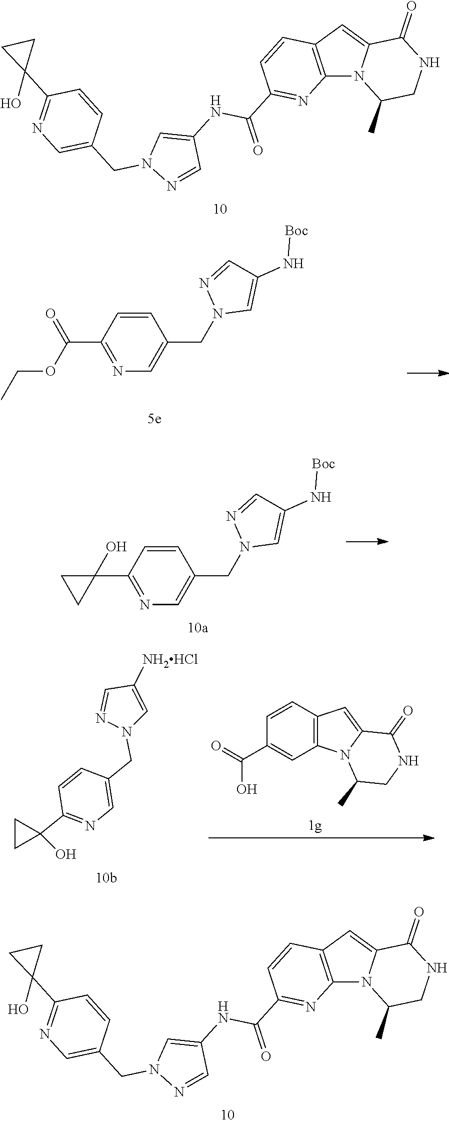

### Step 1 tert-butyl (1-(6-(1-hydroxycyclopropyl) pyridin-3-yl) methyl)-1H-pyrazol-4-yl) aminoformate 10a

At −20° C., EtMgBr (1.6 mL, 1.6 mol) was dissolved in anhydrous THF (5 mL), Ti(OiPr)₄ (206 mg, 0.73 mmol) was slowly added dropwise to the above solution, and stirred for 1 hour, then compound 5e (180 mg, 0.52 mmol) was added, and the reaction was carried out at room temperature for 3 hours. After the reaction was complete, it was quenched with saturated ammonium chloride solution (10 mL), extracted with dichloromethane (50 mL), and washed with saturated saline solution. The organic phase was concentrated under reduced pressure to obtain a crude product, and the crude product was purified by column chromatography (PE/EtOAc=10/1) to obtain compound 10a (50 mg).

MS (ESI) m/z 331 [M+H]⁺.

¹H NMR (400 MHz, CDCl₃) δ 8.65 (d, 1H), 8.07 (d, J=8.06 Hz, 1H), 7.72 (br. s., 1H), 7.62 (dd, J=8.06, 2.15 Hz, 1H), 7.37 (s, 1H), 6.26 (br. s., 1H), 5.28-5.34 (m, 2H), 1.37-1.44 (m, 4H).

### Step 2 1-(5-(4-amino-1H-pyrazol-1-yl) methyl) pyridin-2-yl) cyclopropan-1-ol hydrochloride 10b

Compound 10a (23 mg, 0.07 mmol) was dissolved in methanol (2 mL), and then a 1,4-dioxane solution of hydrochloric acid (2 mL, 4.0 M) was added under nitrogen gas, and the reaction was carried out at room temperature for 3 hours, concentrated under reduced pressure to obtain crude product 10b (25 mg), and which was directly used for the next reaction step.

MS (ESI) m/z 231 [M+H]⁺.

### Step 3 (R)—N-(1-(6-(1-hydroxycyclopropyl) pyridin-3-yl) methyl)-1H-pyrazol-4-yl)-9-methyl-6-oxo-6,7,8,9-tetrahydropyridino [3′, 2′: 4,5]pyrrolo [1,2-a]pyrazine-2-carboxamide 10

Compound 10b (25 mg, 0.07 mmol) was dissolved in DMF (2 mL), then compound 1g (17 mg, 0.07 mmol), HATU (32 mg, 0.08 mmol), and DIPEA (27 mg, 0.21 mmol) were added, and the reaction was carried out at room temperature for 3 hours, then water (30 mL) was added, and extracted with ethyl acetate (20 mL×3). The organic phases were combined, washed with saturated saline water, and concentrated under reduced pressure to obtain a crude product, and the crude product was purified by column chromatography (DCM/MeOH=10/1) to obtain compound 10 (5.3 mg).

MS (ESI) m/z 458 [M+H]⁺.

¹H NMR (400 MHz, CDCl₃) δ 9.67 (s, 1H), 8.60 (d, 1H), 8.29 (s, 1H), 8.22 (d, J=8.19 Hz, 1H), 8.14 (d, J=8.19 Hz, 1H), 8.03 (d, 1H), 7.73-7.66 (m, 2H), 7.31 (s, 1H), 5.41 (s, 2H), 5.29-5.17 (m, 1H), 4.10 (dd, 1H), 3.65-3.53 (m, 1H), 3.23 (q, 2H), 1.58 (m, 3H), 1.22 (t, 4H) ( ).

### Example 11 (R)—N-(1-(6-(1-hydroxycyclobutyl) pyridin-3-yl) methyl)-1H-pyrazol-4-yl)-9-methyl-6-oxo-6,7,8,9-tetrahydropyrido [3′, 2′: 4,5]pyrrolo [1,2-a]pyrazine-2-carboxamide

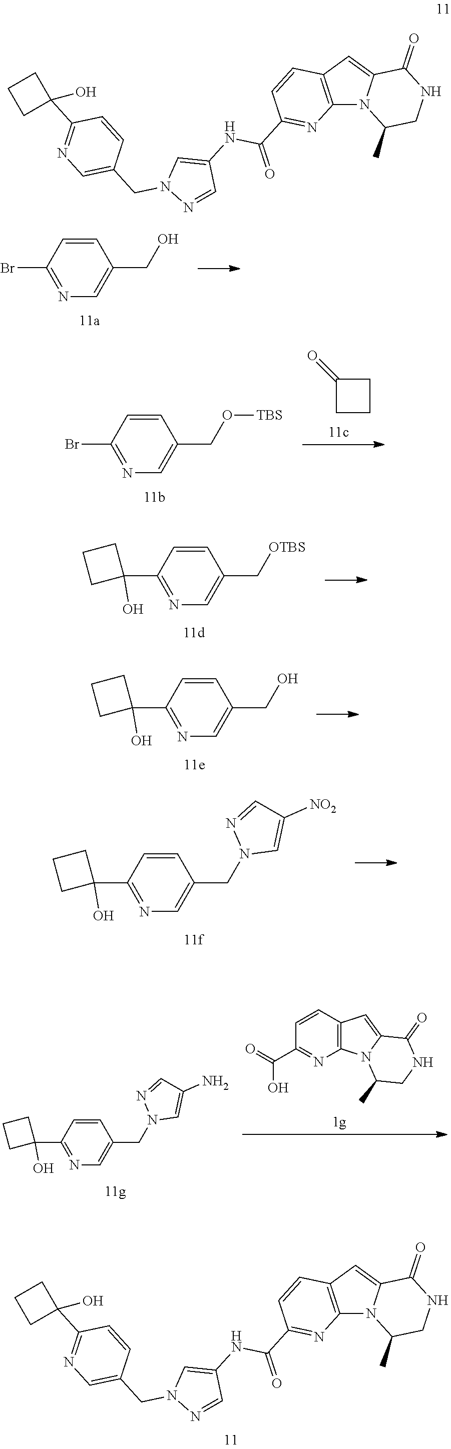

### Step 1 2-bromo-5-((tert butyldimethylsilyl) oxy) methyl) pyridine 11b

Compound 11a (10 g, 53.2 mmol) and imidazole (7.2 g, 106 mmol) were dissolved in dichloromethane (100 mL), and tert-butyldimethylchlorosilane (9.57 g, 63.8 mmol) was added in batches to react at room temperature for 3 hours. To the reaction solution was added saturated ammonium chloride solution (100 mL), and extracted with dichloromethane (250 mL). The organic phase was washed with saturated saline solution (100 mL), concentrated under reduced pressure to obtain a crude product, and the crude product was purified by column chromatography (PE/EtOAc=10/1) to obtain compound 11b (13 g).

¹H NMR (400 MHz, CDCl₃) δ 8.32 (d, 1H), 7.53 (d, 1H), 7.45 (d, 1H), 4.71 (s, 2H), 0.88-0.97 (m, 9H), 0.06-0.13 (m, 6H).

### Step 2 1-(5-((tert-butyldimethylsilyl) oxy) methyl) pyridin-2-yl) cyclobutan-1-ol 11d

Compound 11b (5 g, 16.6 mmol) was dissolved in anhydrous tetrahydrofuran (100 mL), and the reaction system was cooled down to −78° C., butyl lithium (60 mL, 19.9 mmol) was added dropwise, and stirred for additional 20 minutes. Cyclobutanone (11c) (1.39 g, 19.9 mmol) was added dropwise at −78° C. to react at −78° C. for 30 minutes, and then slowly heated up to room temperature to react for 3 hours. After the reaction was completed, the reaction was quenched with added saturated ammonium chloride solution (100 mL), extracted with ethyl acetate (200 mL). The organic phase was washed with saturated saline solution (100 mL) three times, concentrated under reduced pressure to obtain a crude product, and the crude product was purified by column chromatography (PE/EA=10/1) to obtain compound 11d (2.2 g), a colorless oil.

MS (ESI) m/z 294 [M+H]⁺.

¹H NMR (400 MHz, CDCl₃) δ 8.52-8.42 (m, 1H), 7.79 (dd, J=8.06, 2.15 Hz, 1H), 7.57 (d, J=8.06 Hz, 1H), 4.73 (s, 2H), 3.79-3.70 (m, 1H), 2.63-2.40 (m, 2H), 2.16-2.00 (m, 2H), 1.94-1.78 (m, 2H), 0.99-0.88 (m, 9H), 0.15-0.07 (m, 6H)

### Step 3 1-(5-(hydroxymethyl) pyridin-2-yl) cyclobutan-1-ol 11e

Compound 11d (2.0 g, 16.6 mmol) was dissolved in anhydrous dichloromethane (40 mL), and a 1,4-dioxane solution of hydrochloric acid (20 mL, 4.0 M) was added to react for 30 minutes. After the reaction was completed, the spin-dried crude product was purified by column chromatography (PE/EA=10/1) to obtain compound 11e (0.61 g) as a colorless oil.

MS (ESI) m/z 180 [M+H]⁺.

### Step 4 1-(5-(4-nitro-1H-pyrazol-1-yl) methyl) pyridin-2-yl) cyclobutan-1-ol 11f

3-nitropyrazole (536 mg, 4.74 mmol) was dissolved in degassed anhydrous tetrahydrofuran (15 mL), and then compound 11e (567 mg, 3.16 mmol) and triphenylphosphine (2.48 g, 9.48 mmol) were added. Under an ice bath, DBAD (1.09 g, 4.24 mmol) was added in batches and stirred at room temperature for 16 hours. After the reaction was completed, ethyl acetate (20 mL) and saturated sodium bicarbonate solution (10 mL) were added, and extracted with ethyl acetate (20 mL×3), the organic phases were combined, washed with saturated saline solution (50 mL), and concentrated under reduced pressure to obtain a crude product, and the crude product was purified by column chromatography (PE/EtOAc=3/1) to obtain compound 11f (210 mg).

MS (ESI) m/z 275 [M+H]⁺.

¹H NMR (400 MHz, CDCl₃) δ 8.55 (d, 1H), 8.22 (s, 1H), 8.11 (s, 1H), 7.77-7.61 (m, 1H), 7.61-7.52 (m, 1H), 5.38 (s, 2H), 4.13 (d, 1H), 2.66-2.42 (m, 2H), 2.06 (m, 4H).

### Step 5 1-(5-(4-amino-1H-pyrazol-1-yl) methyl) pyridin-2-yl) cyclobutan-1-ol 11g

Compound 11f (150 mg, 0.54 mmol) was dissolved in methanol (5 mL), and then Pd/C and ammonium formate (1 g) were added under nitrogen gas to react at room temperature for 3 hours. After the reaction was completed, it was filtered, and then concentrated under reduced pressure to obtain crude product 11g (60 mg), which was directly used for the next reaction step.

MS (ESI) m/z 245 [M+H]⁺.

### Step 6 (R)—N-(1-(6-(1-hydroxycyclobutyl) pyridin-3-yl) methyl)-1H-pyrazol-4-yl)-9-methyl-6-oxo-6,7,8,9-tetrahydropyrido [3′, 2′: 4,5]pyrrolo [1,2-a]pyrazine-2-carboxamide 11

Compound 11g (50 mg, 0.14 mmol) was dissolved in DMF (2 mL), and then compound 1g (50 mg, 0.16 mmol), HATU (75 mg, 0.16 mmol), and DIPEA (58 mg, 0.45 mmol) were added. The reaction was stirred at room temperature for 3 hours, after the reaction was completed, ethyl acetate (20 mL) and water (10 mL) were added, and extracted with ethyl acetate (20 mL×3). The organic phases were combined, washed with saturated saline (50 mL), and concentrated under reduced pressure to obtain a crude product, and the crude product was purified by column chromatography (DCM/MeOH=10/1) to obtain compound 11 (36.8 mg).

MS (ESI) m/z 472 [M+H]⁺.

¹H NMR (400 MHz, CDCl₃) δ 10.64 (s, 1H), 8.51 (d, J=1.75 Hz, 1H), 8.35-8.25 (d, 2H), 7.92 (d, J=8.19 Hz, 1H), 7.80 (s, 1H), 7.66 (dd, J=8.19, 1H), 7.55 (d, 1H), 7.13 (s, 1H), 5.71 (s, 1H), 5.38 (s, 2H), 5.28 (br. s., 1H), 3.91 (dd, 1H), 3.48 (d, J=6.72 Hz, 1H), 3.10 (m, 1H), 2.26-2.13 (m, 2H), 1.87 (s, 2H), 1.79 (s, 2H), 1.42 (d, J=6.45 Hz, 3H).

### Example 12 (8S, 9R)—N-(1-(6-(2-hydroxyprop-2-yl) pyridin-3-yl) methyl)-1H-pyrazol-4-yl)-8,9-dimethyl-6-oxo-6,7,8,9-tetrahydropyridino [3′,2′: 4,5]pyrrolo [1,2-a]pyrazine-2-carboxamide

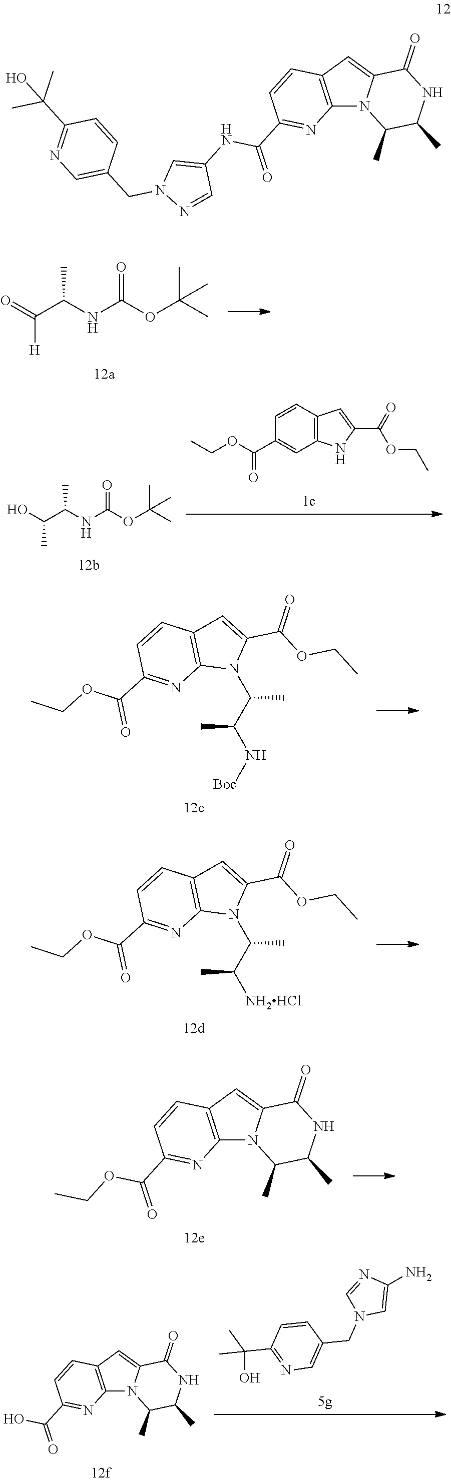

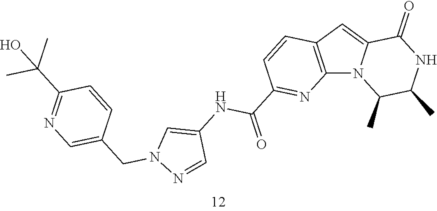 [SMILES: C[C@@H]1NC(=O)c2cc3ccc(C(=O)Nc4cnn(Cc5ccc(C(C)(C)O)nc5)c4)nc3n2[C@@H]1C]

### Step 1 (2S, 3S)-3-hydroxybut-2-yl) aminoformate tert butyl ester 12b

Compound 12a (3.83 g, 22.1 mmol) was dissolved in anhydrous tetrahydrofuran (100 mL), and then Me₂CuLi (97 mL, 48.6 mmol, 0.5M in ether) was added at −55° C. to react at this temperature for 4 hours. Then, the reaction was carried out for 3 hours under an ice bath, then saturated ammonium chloride solution (20 mL) was added, and extracted with ethyl acetate (50 mL×3). The organic phases were combined, washed with saturated saline solution (20 mL), and concentrated under reduced pressure to obtain a crude product, and the crude product was purified by column chromatography (PE/EtOAc=3/1) to obtain compound 12b (2.3 g).

MS (ESI) m/z 190 [M+H]⁺

### Step 2 diethyl 1-((2R, 3S)-3-(tert-butoxycarbonyl) amino) but-2-yl)-1H pyrrolo [2,3-b]pyridine-2,6-dicarboxylate 12c

Compound 12b (2.3 g, 12.3 mmol) was dissolved in anhydrous tetrahydrofuran (30 mL), and then compound 1c (3.98 g, 15.2 mmol) and triphenylphosphine (7.97 g, 30.4 mmol) were added. The reaction was placed in an ice bath, then DBAD (5.24 g, 22.8 mmol) was added in batches, and stirred at room temperature for 16 hours. After the reaction was completed, ethyl acetate (50 mL) and saturated sodium bicarbonate solution (10 mL) were added, and extracted with ethyl acetate (20 mL×3). The organic phases were combined, washed with saturated saline solution (10 mL), and concentrated under reduced pressure to obtain a crude product, and the crude product was purified by column chromatography (PE/EtOAc=2/1) to obtain compound 12c (1.8 g).

MS (ESI) m/z 434 [M+H]⁺

¹H NMR (400 MHz, CDCl₃) δ 8.15-8.02 (m, 1H), 8.01-7.90 (m, 1H), 7.29 (d, 1H), 4.55-4.34 (m, 4H), 1.84 (d, 1H), 1.68 (s, 1H), 1.55-1.45 (m, 15H), 1.45-1.38 (m, 6H).

### Step 3 diethyl 1-((2R, 3S)-3-aminobut-2-yl)-1H pyrrolo [2,3-b]pyridine-2,6-dicarboxylate hydrochloride 12d

Compound 12c (0.14 g, 0.32 mmol) was dissolved in methanol (10 mL), then a 1,4-dioxane solution of hydrochloric acid (5 mL, 4.0 M) was added, and the reaction was stirred at room temperature for 3 hours. After the reaction was complete, it was concentrated under reduced pressure to obtain crude product 12d (140 mg), which was directly used for the next reaction step.

MS (ESI) m/z 334 [M+H]⁺.

### Step 4 (8S, 9R)-8,9-dimethyl-6-oxo-6,7,8,9-tetrahydropyridino [3′, 2′: 4,5]pyrrolo [1,2-a]pyrazine-2-carboxylate 12e

Compound 12d (140 mg, 0.32 mmol) was dissolved in ethanol (5 mL), then potassium carbonate (88 mg, 0.64 mmol) was added, and the reaction was stirred at 70° C. for 3 hours. After the reaction was completed, it was filtered and concentrated under reduced pressure to obtain a crude product, and the crude product was purified by column chromatography (DCM/MeOH=10/1) to obtain compound 12e (0.3 g).

MS (ESI) m/z 288 [M+H]⁺.

¹H NMR (400 MHz, CDCl₃) δ 8.14 (d, 1H), 7.99 (d, 1H), 7.26 (s, 1H), 5.21-5.03 (m, 1H), 4.63-4.38 (m, 2H), 4.26 (dd, 1H), 1.47 (s, 3H), 1.42 (d, 3H), 1.36 (d, 3H).

### Step 5 (8S, 9R)-8,9-dimethyl-6-oxo-6,7,8,9-tetrahydropyridino [3′, 2′: 4,5]pyrrolo [1,2-a]pyrazine-2-carboxylic acid 12f

Compound 12e (45 mg, 0.15 mmol) was dissolved in tetrahydrofuran (2 mL) and water (2 mL), and then lithium hydroxide monohydrate (19 mg, 0.3 mmol) was added to react at room temperature for 3 hours. After the reaction was completed, it was concentrated under reduced pressure to obtain compound 12f (45 mg), which was directly used for the next step.

MS (ESI) m/z 260 [M+H]⁺.

### Step 6 (8S, 9R)—N-(1-(6-(2-hydroxyprop-2-yl) pyridin-3-yl) methyl)-1H-pyrazol-4-yl)-8,9-dimethyl-6-oxo-6,7,8,9-tetrahydropyridino [3′, 2′: 4,5]pyrrolo [1,2-a]pyrazine-2-carboxamide 12

Compound 12f (40 mg, 0.15 mmol) was dissolved in DMF (2 mL), then compound 5g (30 mg, 0.12 mmol), HATU (71 mg, 0.18 mmol), and DIPEA (60 mg, 0.46 mmol) were added to react at room temperature for 3 hours. After the reaction was completed, ethyl acetate (20 mL) and water (10 mL) were added, and extracted with ethyl acetate (20 mL×3). The organic phases were combined, washed with saturated saline water (50 mL), and concentrated under reduced pressure to obtain a crude product, and the crude product was purified by column chromatography (DCM/MeOH=10/1) to obtain compound 12 (2.6 mg).

MS (ESI) m/z 474 [M+H]⁺.

¹H NMR (400 MHz, CDCl₃) δ 9.57 (s, 1H), 8.42 (s, 1H), 8.18 (s, 1H), 8.13 (d, 1H), 8.06 (d, 1H), 7.62 (s, 1H), 7.57 (dd, 1H), 7.31 (d, 1H), 5.59 (s, 1H), 5.35 (s, 2H), 5.01 (s, 1H), 5.00-4.83 (m, 1H), 4.23 (dd, 1H), 2.02-1.86 (m, 6H), 1.47 (m, 6H).

### Example 13 (R)-6-fluoro-N-(1-(6-(2-hydroxypropan-2-yl) pyridin-3-yl) methyl)-1H-pyrazol-4-yl)-4-methyl-1-oxo-1,2,3,4-tetrahydropyrazino [1,2-a]indole-7-carboxamide

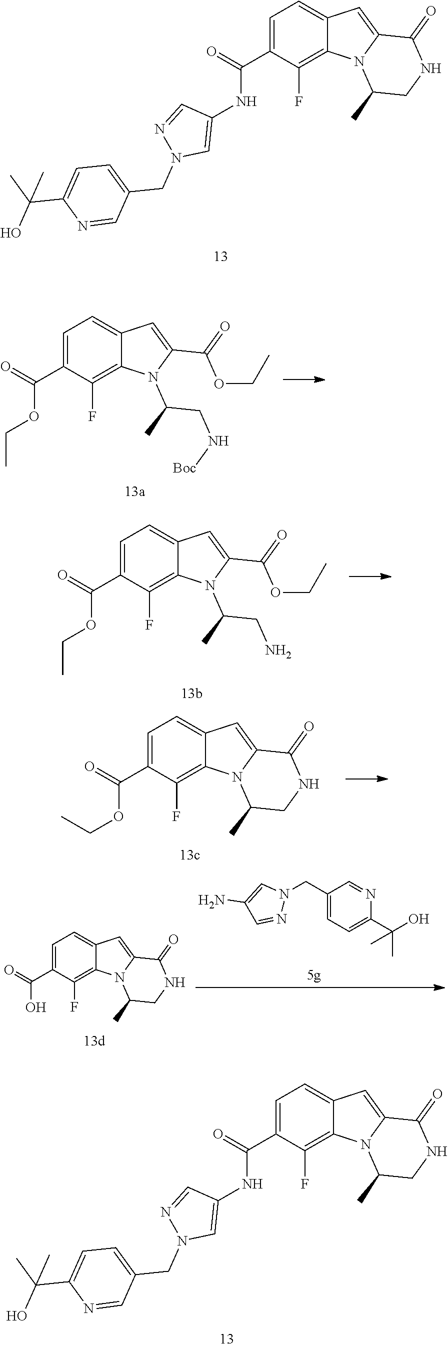

### Step 1 diethyl (R)-1-(1-aminopropyl-2-yl)-7-fluoro-1H-indole-2,6-dicarboxylate 13b

Compound 13a (335 mg, 0.77 mmol) was dissolved in a dichloromethane solution (6 mL), and then trifluoroacetic acid (2 mL) was added dropwise and stirred at room temperature for 3 hours. After the reaction was completed, it was concentrated under reduced pressure, then saturated sodium bicarbonate solution (100 mL) was added, and extracted with ethyl acetate (50 mL×3), and purified by silica gel normal phase chromatography to obtain compound 13b (258 mg), a colorless oily liquid.

### Step 2 (R)-6-fluoro-4-methyl-1-oxo-1,2,3,4-tetrahydropyrazino [1,2-a]indole-7-carboxylate 13c

Compound 13b (258 mg, 0.77 mmol) was dissolved in ethanol (10 mL), and potassium carbonate (428 mg, 2.30 mmol) was added and heated tp to 60° C. to react for 3 hours, after TLC monitored the completion of the reaction, it was concentrated under reduced pressure, extracted with ethyl acetate (50 mL×3), the organic phases were combined and washed with saturated saline (50 mL), and the obtained crude product was purified by silica gel normal phase chromatography to obtain compound 13c (225 mg), a white solid.

¹H NMR (400 MHz, CDCl₃) δ 7.57 (dd, J=8.5, 6.3 Hz, 1H), 7.39 (d, J=8.5 Hz, 1H), 7.27 (d, J=5.3 Hz, 1H), 7.20 (d, J=2.1 Hz, 1H), 5.11-5.01 (m, 1H), 4.36 (qd, J=7.1, 1.1 Hz, 2H), 4.04 (dd, J=12.9, 4.4 Hz, 1H), 3.46 (ddd, J=12.7, 5.4, 1.3 Hz, 1H), 1.47 (d, J=6.6 Hz, 3H), 1.36 (t, J=7.1 Hz, 3H).

### Step 3 (R)-6-fluoro-4-methyl-1-oxo-1,2,3,4-tetrahydropyrazino [1,2-a]indole-7-carboxylic acid 13d

Compound 13c (187 mg) was dissolved in 1,4-dioxane (6 mL), and then lithium hydroxide (19 mg, 0.77 mmol) was added slowly and stirred at room temperature for 1 hour, after TLC monitored the completion of the reaction, it was concentrated under reduced pressure until a white solid was precipitated, and then filtered to obtain compound 13d (white solid, 148 mg, 46%).

MS (ESI) m/z 261 [M−H]⁺.

### Step 4 (R)-6-fluoro-N-(1-(6-(2-hydroxypropan-2-yl) pyridin-3-yl) methyl)-1H-pyrazol-4-yl)-4-methyl-1-oxo-1,2,3,4-tetrahydropyrazino [1,2-a]indole-7-carboxamide 13

Under argon gas protection, compound 13d (25 mg, 0.095 mmol), compound 5g (24 mg, 0.11 mmol), PyBop (74 mg, 0.14 mmol), DIPEA (37 mg, 0.29 mmol), and DMF (3 mL) were sequentially added to a 50 mL three necked flask, and the reaction was carried out at room temperature for 24 hours. After TLC monitored the completion of the reaction, water was added to the reaction system, and extracted with ethyl acetate (50 mL×3). The organic phases were combined, washed with saturated saline solution, and concentrated under reduced pressure to obtain a crude product, and the crude product was purified by column chromatography to obtain compound 13 (18 mg), a white solid.

MS (ESI) m/z 477 [M+H]⁺.

¹H NMR (400 MHz, CD3OD) δ 8.34 (t, J=1.5 Hz, 1H), 8.11 (s, 1H), 7.64-7.56 (m, 3H), 7.46 (d, J=8.4 Hz, 1H), 7.30 (dd, J=8.4, 6.1 Hz, 1H), 7.15 (d, J=2.2 Hz, 1H), 5.30 (s, 2H), 5.08-5.00 (m, 1H), 3.94 (dd, J=13.2, 4.5 Hz, 1H), 3.43 (dd, J=13.2, 1.3 Hz, 1H), 1.43 (s, 6H), 1.41 (d, J=6.5 Hz, 3H).

### Example 14 (S)—N-(1-(6-(2-hydroxyprop-2-yl) pyridin-3-yl) methyl)-1H-pyrazol-4-yl)-8-methyl-6-oxo-6,7,8,9-tetrahydropyridino [3 2′: 4,5]pyrrolo [1,2-a]pyrazine-2-carboxamide

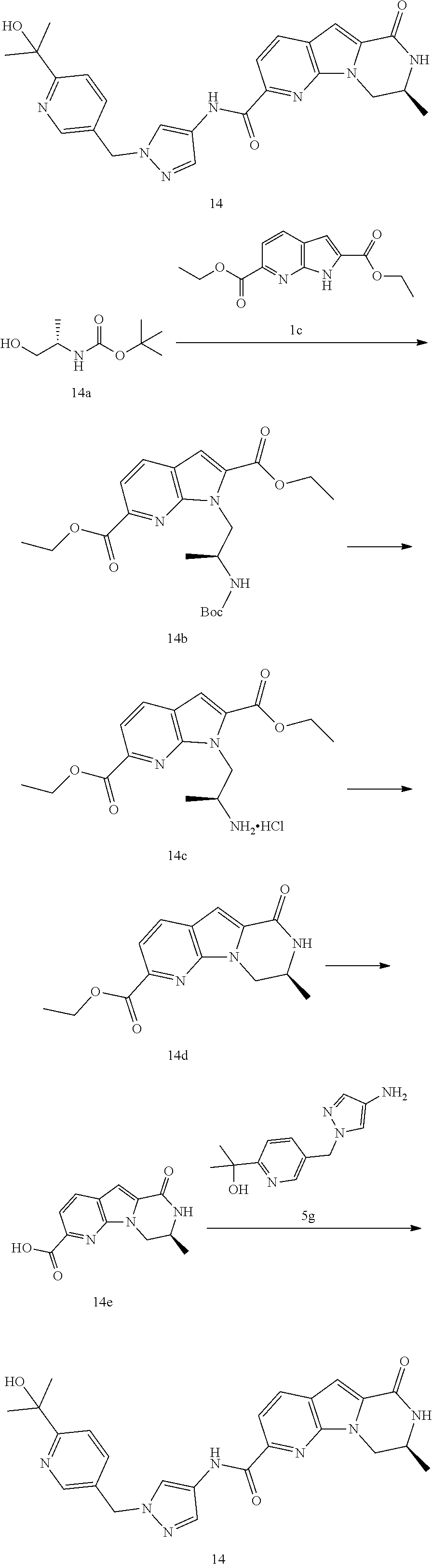

### Step 1 diethyl (S)-1-(2-(tert butoxycarbonyl) amino) propyl)-1H pyrrolo [2,3-b]pyridine-2,6-dicarboxylate 14b

Compound 14a (0.4 g, 2.3 mmol) was dissolved in anhydrous tetrahydrofuran (10 mL), and compound 1c (0.5 g, 1.9 mmol) and triphenylphosphine (1.25 g, 4.75 mmol) were added. The reaction was replaced in an ice bath, and DBAD (0.87 g, 3.8 mmol) was added in batches and stirred at room temperature for 16 hours. After TLC monitored the completion of the reaction, ethyl acetate (50 mL) and saturated sodium bicarbonate solution (10 mL) were added, and it was extracted with ethyl acetate (20 mL×3), and the organic phases were combined, washed with saturated saline solution (10 mL), and concentrated under reduced pressure to obtain a crude product, and the crude product was purified by column chromatography (PE/EtOAc=5/1) to obtain compound 14b (500 mg).

MS (ESI) m/z 420 [M+H]⁺

¹H NMR (400 MHz, CDCl₃) δ 8.07 (d, 1H), 7.96 (d, 1H), 7.29 (s, 1H), 6.23 (br. s., 1H), 4.81 (d, 2H), 4.57-4.35 (m, 4H), 4.25-4.17 (m, 1H), 1.53-1.45 (m, 12H), 1.34-1.21 (m, 6H).

### Step 2 diethyl (S)-1-(2-aminopropyl)-1H pyrrolo [2,3-b]pyridine-2,6-dicarboxylate hydrochloride 14c

Compound 14b (0.5 g, 1.2 mmol) was dissolved in methanol (5 mL), then a 1,4-dioxane solution of hydrochloric acid (5 mL, 4.0 M) was added, and the reaction was stirred at room temperature for 3 hours, after TLC monitored the completion of the reaction, it was concentrated under reduced pressure to obtain crude product 14c (500 mg), which was used directly for the next reaction step.

MS (ESI) m/z 320 [M+H]⁺.

### Step 3 (S)-8-methyl-6-oxo-6,7,8,9-tetrahydropyridino [3′, 2′: 4,5]pyrrolo [1,2-a]pyrazine-2-carboxylate 14d

Compound 14c (500 mg, 1.2 mmol) was dissolved in ethanol (5 mL), and then potassium carbonate (331 mg, 2.4 mmol) was added to react at 70° C. for 3 hours, after TLC monitored the completion of the reaction, it was filtered, and concentrated under reduced pressure to obtain a crude product, and then the crude product was purified by column chromatography (DCM/MeOH=10/1) to obtain compound 14d (0.3 g).

MS (ESI) m/z 274 [M+H]⁺.

### Step 4 (S)-8-methyl-6-oxo-6,7,8,9-tetrahydropyridino [3′, 2′: 4,5]pyrrolo [1,2-a]pyrazine-2-carboxylic acid 14e

Compound 14d (208 mg, 0.76 mmol) was dissolved in tetrahydrofuran (5 mL) and water (5 mL), and then lithium hydroxide monohydrate (94 mg, 2.28 mmol) was added to react at room temperature for 3 hours, after TLC monitored the completion of the reaction, it was filtered, and then concentrated under reduced pressure to obtain crude product 14e (0.3 g), which was directly used for the next reaction step.

MS (ESI) m/z 246 [M+H]⁺.

### Step 5 (S)—N-(1-(6-(2-hydroxyprop-2-yl) pyridin-3-yl) methyl)-1H-pyrazol-4-yl)-8-methyl-6-oxo-6,7,8,9-tetrahydropyridino [3′, 2′: 4,5]pyrrolo [1,2-a]pyrazine-2-carboxamide 14

Compound 14e (30 mg, 0.12 mmol) was dissolved in DMF (2 mL), and then compound 5g (30 mg, 0.12 mmol), HATU (46 mg, 0.12 mmol), and DIPEA (46 mg, 0.36 mmol) were added to react at room temperature for 3 hours, after TLC monitored the completion of the reaction, ethyl acetate (20 mL) and water (10 mL) were added, and extracted with ethyl acetate (20 mL×3). The organic phases were combined, washed with saturated saline solution (50 mL), and concentrated under reduced pressure to obtain a crude product, and the crude product was purified by column chromatography (DCM/MeOH=10/1) to obtain compound 14 (52 mg).

MS (ESI) m/z 460 [M+H]⁺.

¹H NMR (400 MHz, CDCl₃) δ 8.59 (s, 2H), 8.46-8.39 (m, 2H), 8.05 (d, 1H), 7.93 (s, 1H), 7.85-7.70 (m, 2H), 5.49 (s, 2H), 5.33 (s, 1H), 4.93 (d, 1H), 4.28-4.13 (m, 2H), 1.59-1.51 (m, 6H), 1.45 (d, 3H).

### Example 15 (R)—N-(1-((5-fluoro-6-(2-hydroxyprop-2-yl) pyridin-3-yl) methyl)-1H-pyrazol-4-yl)-9-methyl-6-oxo-6,7,8,9-tetrahydropyridino [3, 2′: 4,5]pyrrolo [1,2-a]pyrazine-2-carboxamide

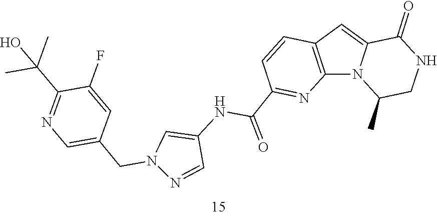 [SMILES: C[C@@H]1CNC(=O)c2cc3ccc(C(=O)Nc4cnn(Cc5cnc(C(C)(C)O)c(F)c5)c4)nc3n21]

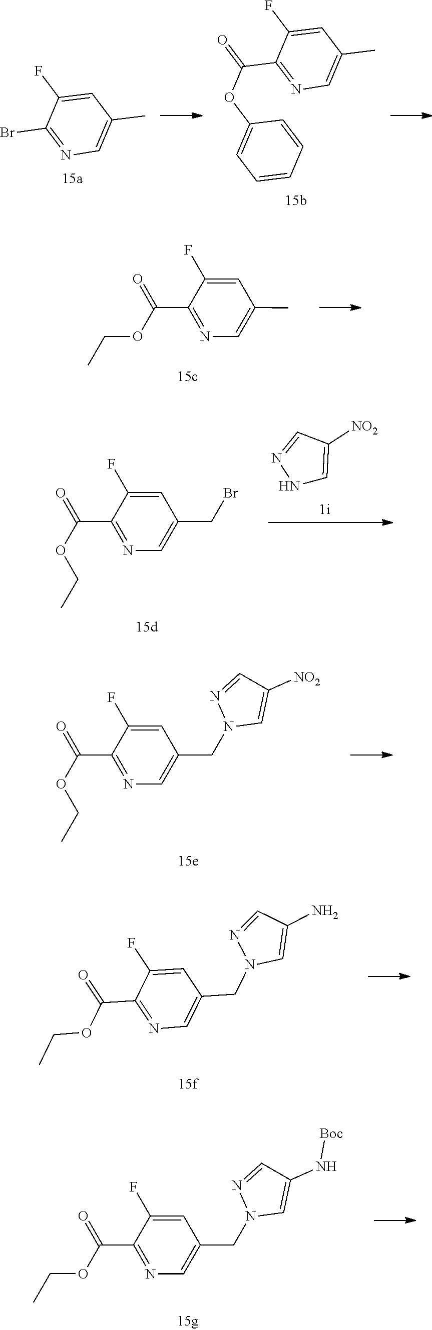

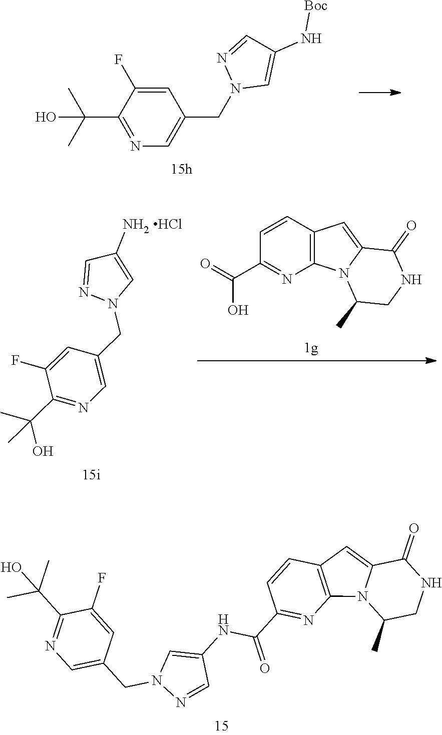

### Step 1 phenyl 3-fluoro-5-methyl pyridinecarboxylate 15b

Compound 15a (5 g, 26.3 mmol) was dissolved in anhydrous acetonitrile (50 mL), and benzyl formate (4.17 g, 34.2 mmol), triethylamine (6.6 g, 19.5 mmol), Pd(OAc)₂ (217 mg, 1.31 mmol), and Xantphos (1.1 g, 2.62 mmol) were added. The reaction was carried out at 80° C. for 4 hours, then cooled down to room temperature, and saturated ammonium chloride solution (20 mL) was added, and extracted with ethyl acetate (250 mL×3). The organic phases were combined, washed with saturated saline solution (200 mL), and concentrated under reduced pressure to obtain a crude product, and the crude product was purified by column chromatography (PE/EtOAc=5/1) to obtain compound 15b (3.6 g).

¹H NMR (400 MHz, CDCl₃) δ 8.47 (s, 1H), 7.48-7.39 (m, 3H), 7.34-7.22 (m, 4H), 2.49 (s, 3H).

### Step 2 ethyl 3-fluoro-5-methyl pyridinecarboxylate 15c

Compound 15b (3.52 g, 15.2 mmol) was dissolved in anhydrous ethanol (30 mL), and sodium ethoxide (0.1 g, 1.5 mmol) was added and stirred at room temperature for 16 hours. After TLC monitored the completion of the reaction, water (50 mL) was added, and extracted with ethyl acetate (20 mL×3). The organic phases were combined, washed with saturated saline solution (10 mL), and concentrated under reduced pressure to obtain a crude product, and the crude product was purified by column chromatography (PE/EtOAc=2/1) to obtain compound 15c (2.2 g).

MS (ESI) m/z 184 [M+H]⁺

### Step 3 ethyl 5-(bromomethyl)-3-fluoropyridinecarboxylate 15d

Compound 15c (2.1 g, 11.5 mmol) was dissolved in 1,2-dichloroethane (40 mL), and N-bromosuccinimide (2.6 g, 14.9 mmol) was added, and AIBN (0.18 g, 1.15 mmol) was added in batches at 85° C. After TLC monitored the completion of the reaction, it was concentrated under reduced pressure to obtain a crude product, and the crude product was purified by column chromatography (PE/EtOAc=3/1) to obtain compound 15d (1.2 g).

¹H NMR (400 MHz, CDCl₃) δ 8.57 (d, 1H), 7.80 (d, 1H), 4.58-4.40 (m, 2H), 2.45-2.26 (m, 3H), 1.52-1.36 (m, 3H).

### Step 4 ethyl 3-fluoro-5-(4-nitro-1H-pyrazol-1-yl) methyl) pyridinecarboxylate 15e

Compound 15d (2 g, 7.6 mmol) was dissolved in acetonitrile (30 mL), and 4-nitropyrazole (858 mg, 7.6 mmol), potassium carbonate (1.46 g, 15 mmol) were added to react at 70° C. for 3 hours. After TLC monitored the completion of the reaction, ethyl acetate (30 mL) was added, filtered and concentrated under reduced pressure to obtain a crude product, and the crude product was purified by column chromatography (DCM/MeOH=10/1) to obtain compound 15e (0.8 g).

MS (ESI) m/z 295 [M+H]⁺.

### Step 5 ethyl 5-((4-amino-1H-pyrazol-1-yl) methyl)-3-fluoropyridinecarboxylate 15f

Compound 15e (800 mg, 0.54 mmol) was dissolved in methanol (50 mL), Pd/C and ammonium formate (3 g) were added under nitrogen gas, and then the reaction was stirred at room temperature for 3 hours. After TLC monitored the completion of the reaction, it was filtered, and then concentrated under reduced pressure to obtain crude product 15f (800 mg), which was directly used for the next reaction step.

MS (ESI) m/z 265 [M+H]⁺.

### Step 6 ethyl 5-((4-(tert-butoxycarbonyl) amino)-1H-pyrazol-1-yl) methyl)-3-fluoropyridinecarboxylate 15g

Compound 15f (800 mg, 0.54 mmol) was dissolved in DCM (20 mL), and triethylamine (110 mg, 1.1 mmol) and di-tert-butyl dicarbonate (130 mg, 0.6 mmol) were added, and then the reaction was stirred at room temperature for 3 hours. After TLC monitored the completion of the reaction, water (10 mL) was added, extracted with ethyl acetate (20 mL×3). The organic phases were combined, washed with saturated saline water (50 mL), and concentrated under reduced pressure to obtain a crude product, and the crude product was purified by column chromatography (DCM/MeOH=10/1) to obtain compound 15g (0.5 g).

MS (ESI) m/z 365 [M+H]⁺.

¹H NMR (400 MHz, CDCl₃) δ 8.43 (s, 1H), 7.76 (br. s., 1H), 7.63 (br. s., 1H), 7.38 (s, 1H), 7.30 (dd, 1H), 5.32 (s, 2H), 4.48 (q, 2H), 1.55-1.46 (m, 9H), 1.46-1.38 (m, 3H).

### Step 7 tert-butyl 1-(5-fluoro-6-(2-hydroxypropan-2-yl) pyridin-3-yl) methyl)-1H-pyrazol-4-yl) carbamate 15 h

Compound 15g (0.5 g, 1.37 mmol) was dissolved in THF (10 mL), and methyl magnesium bromide (5.48 mL, 5.48 mmol) was added dropwise under an ice bath, and the reaction was carried out at room temperature for 3 hours. Saturated ammonium chloride solution (10 mL) was added to the reaction solution to quench the reaction, and then the reaction was extracted with dichloromethane (25 mL), and the organic phase was washed with saturated saline solution (100 mL) and concentrated under reduced pressure to obtain a crude product, and the crude product was purified by column chromatography (PE/EtOAc=10/1) to obtain compound 15h (0.25 g).

MS (ESI) m/z 351 [M+H]⁺.

### Step 8 2-(5-((4-amino-1H-pyrazol-1-yl) methyl)-3-fluoropyridin-2-yl) propan-2-ol hydrochloride 15i

Compound 15h (250 mg, 0.71 mmol) was dissolved in methanol (5 mL), and a 1,4-dioxane solution of hydrochloric acid (5 mL, 4 M) was added under nitrogen gas, and then stirred at room temperature for 3 hours, after TLC monitored the completion of the reaction, it was concentrated under reduced pressure to obtain a crude product, and then compound 15i (800 mg) was obtained, which was directly used for the next reaction step.

MS (ESI) m/z 251 [M+H]⁺.

### Step 9 (R)—N-(1-(5-fluoro-6-(2-hydroxyprop-2-yl) pyridin-3-yl) methyl)-1H-pyrazol-4-yl)-9-methyl-6-oxo-6,7,8,9-tetrahydropyridino [3′, 2′: 4,5]pyrrolo [1,2-a]pyrazine-2-carboxamide 15

Compound 15i (250 mg, 1.0 mmol) was dissolved in DMF (5 mL), and compound 1g (254 mg, 1.0 mmol), HATU (380 mg, 1.0 mmol), and DIPEA (390 mg, 3.0 mmol) were added. The reaction was stirred at room temperature for 3 hours, after TLC monitored the completion of the reaction, water (10 mL) was added, and extracted with ethyl acetate (20 mL×3). The organic phases were combined, washed with saturated saline solution (50 mL), and concentrated under reduced pressure to obtain a crude product, and the crude product was purified by column chromatography (DCM/MeOH=10/1) to obtain compound 15 (31.7 mg).

MS (ESI) m/z 478 [M+H]⁺.

¹H NMR (400 MHz, CDCl₃) δ 9.69 (s, 1H), 8.30 (s, 2H), 8.21 (d, 1H), 8.14 (d, 1H), 7.70 (s, 1H), 7.34-7.28 (m, 2H), 6.57 (d, 1H), 5.37 (s, 2H), 5.28-5.13 (m, 1H), 4.09 (dd, 1H), 3.59 (dd, 1H), 1.62-1.47 (m, 9H).

### Example 16 (8S, 9R)—N-(1-((5-fluoro-6-(2-hydroxyprop-2-yl) pyridin-3-yl) methyl)-1H-pyrazol-4-yl)-8,9-dimethyl-6-oxo-6,7,8,9-tetrahydropyridino [3′, 2′: 4,5]pyrrolo [1,2-a]pyrazine-2-carboxamide

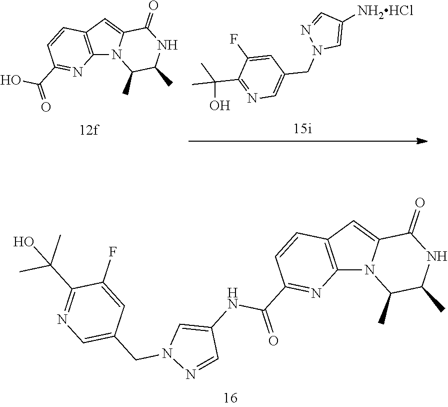

Compound 12f (100 mg, 0.38 mmol) was dissolved in DMF (2 mL), and compound 15i (128 mg, 0.36 mmol), HATU (164 mg, 0.43 mmol), and DIPEA (129 mg, 1.08 mmol) were added to react at room temperature for 3 hours. After TLC monitored the completion of the reaction, water (10 mL) was added, and extracted with ethyl acetate (20 mL×3). The organic phases were combined, washed with saturated saline water (30 mL), and concentrated under reduced pressure to obtain a crude product, and the crude product was purified by column chromatography (DCM/MeOH=10/1) to obtain compound 16 (6.8 mg).

MS (ESI) m/z 492 [M+H]⁺.

¹H NMR (400 MHz, CDCl₃) δ 9.67 (s, 1H), 8.31 (s, 1H), 8.18 (dd, 2H), 7.70 (s, 1H), 7.31 (d, 2H), 5.60 (s, 1H), 5.45 (s, 1H), 5.36 (s, 2H), 5.03 (m, 1H), 4.33-4.30 (m, 1H), 1.57 (s, 6H), 1.49-1.47 (d, 3H), 1.41-1.44 (d, 3H).

### Example 17 (R)—N-(1-(6-(2-hydroxypropan-2-yl-1,1,3,3-d₆) pyridin-3-yl) methyl)-1H-pyrazol-4-yl)-9-methyl-6-oxo-6,7,8,9-tetrahydropyridino [3′, 2′: 4,5]pyrrolo [1,2-a]pyrazine-2-carboxamide

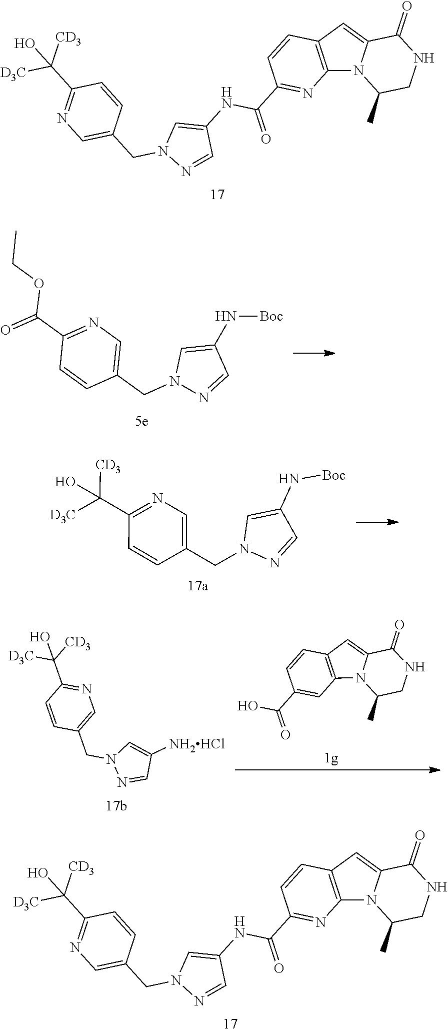

### Step 1 tert butyl (1-(6-(2-hydroxypropan-2-yl-1,1,1,3-d6) pyridin-3-yl) methyl)-1H-pyrazol-4-yl) aminoformate 17a

Compound 5e (0.22 g, 0.63 mmol) was dissolved in THF (10 mL), and CD₃MgBr (2.86 mL, 2.86 mmol) was added dropwise under an ice bath, and then the reaction was carried out at room temperature for 3 hours. After the reaction was completed, the reaction was quenched with saturated ammonium chloride solution (10 mL), extracted with dichloromethane (25 mL), and washed with saturated saline solution. The organic phase was concentrated under reduced pressure to obtain a crude product, and the crude product was purified by column chromatography (PE/EtOAc=10/1) to obtain compound 17a (0.1 g).

MS (ESI) m/z 339 [M+H]⁺.

### Step 2 2-(5-((4-amino-1H-pyrazol-1-yl) methyl) pyridin-2-yl) propyl-1,1,3,3,3-d₆-2-0l hydrochloride 17b

Compound 17a (250 mg, 0.71 mmol) was dissolved in methanol (5 mL), and a 1,4-dioxane solution of hydrochloric acid (5 mL, 4.0 M) was added under nitrogen gas, and then the reaction was carried out at room temperature for 3 hours, after TLC monitored the completion of the reaction, it was concentrated under reduced pressure to obtain a crude product, and then compound 17b (100 mg) was obtained, which was directly used for the next reaction step.

MS (ESI) m/z 239 [M+H]⁺.

### Step 3 (R)—N-(1-(6-(2-hydroxypropan-2-yl-1,1,3,3-d6) pyridin-3-yl) methyl)-1H-pyrazol-4-yl)-9-methyl-6-oxo-6,7,8,9-tetrahydropyridino [3′, 2′: 4,5]pyrrolo [1,2-a]pyrazine-2-carboxamide 17

Compound 17b (76 mg, 0.32 mmol) was dissolved in DMF (5 mL), and compound 1g (93 mg, 0.38 mmol), HATU (144 mg, 0.38 mmol), and DIPEA (147 mg, 1.1 mmol) were added, and then the reaction was stirred at room temperature for 3 hours, after TLC monitored the completion of the reaction, water (10 mL) was added, and extracted with ethyl acetate (20 mL×3). The organic phases were combined, washed with saturated saline solution (50 mL), and concentrated under reduced pressure to obtain a crude product, and the crude product was purified by column chromatography (DCM/MeOH=10/1) to obtain compound 17 (28.4 mg).

MS (ESI) m/z 466 [M+H]+

¹H NMR (400 MHz, CDCl₃) δ 9.66 (s, 1H), 8.49 (d, 1H), 8.26 (s, 1H), 8.22 (d, 1H), 8.14 (d, 1H), 7.68 (s, 1H), 7.63 (dd, 1H), 7.37 (d, 1H), 7.31 (s, 1H), 6.13 (d, 1H), 5.35 (s, 2H), 5.18-5.27 (m, 1H), 4.81 (s, 1H), 4.10 (dd, 1H), 3.57 (m, 1H), 1.62 (m, 3H).

### Conrol Compound I (BI-D1870)

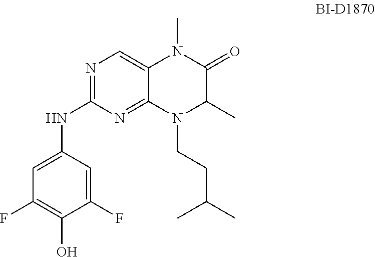 [SMILES: CC(C)CCN1c2nc(Nc3cc(F)c(O)c(F)c3)ncc2N(C)C(=O)C1C]

This compound was purchased commercially, manufacturer: MCE, item number: HY-10510

### Conrol Compound II

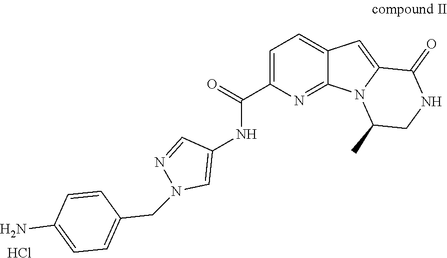 [SMILES: C[C@@H]1CNC(=O)c2cc3ccc(C(=O)Nc4cnn(Cc5ccc(N)cc5)c4)nc3n21 ; Cl]

It was prepared according to the method described in WO2017141116A1.

### Test Example 1: IC₅₀ Evaluation Test of Compound Inhibition on RSK2 Kinase Activity in Vitro

The inhibitory effect of small molecule compounds on RSK2 kinase was detected using HTRF KinEASE-STK assay. The RSK2 kinase reaction system is 5 uL, and the final concentrations of each component are as follows: 0.18 nM RSK2 recombinase (Carna, Cat. NO. 01-150), small molecule inhibitors with different concentration gradients, 5 mM MgCl₂, 1 mM DTT, 10 μM ATP, 1 M STK1 Substrate (Cisbio, Cat. NO. 61ST1BLC), 1× kinase reaction buffer (Cisbio, Cat. NO. #62EZBFDD). Specifically, firstly, 2.5×RSK2 recombinase and 5×inhibitor were added to a buffer system in a 384 well plate, and incubated at room temperature for 10 minutes, negative control wells without enzyme and positive control wells without compounds were set at the same time, then, 2.5×peptide substrate and ATP were added, and then the reaction began. After 30 minutes of incubation at room temperature, a mixture of 5 μL corresponding 2×phosphorylated antibody STK antibody Cryptate (Cisbio, Cat NO. 62STOPEB) and substrate labeled antibody Sa-XL 665 (Cisbio, Cat NO. 610SAXLA) was added to terminate the reaction, and then it was left at room temperature for 60 minutes. The values of excitation at 320 nm, emission at 620 nm and 665 nm were read on the HTS high-throughput drug screening multifunctional enzyme-linked immunosorbent assay reader, RFU 620 nm/RFU 665 nm was calculated, and the inhibition rate was calculated using the following formula, and then the curve was plotted with Log value of the inhibitor concentration as the X-axis and inhibition rate as the Y-axis, and the IC₅₀ value was calculated using Graphpad 7.0.

$$Inhibition \% = [ 1 - \frac{{Data}_{Test Compound} - {\overline{Data}}_{Positive Control}}{{\overline{Data}}_{Negative Control} - {\overline{Data}}_{Positive Control}} ] \times 100$$

D̅a̅t̅a̅_{Positive Control}: mean value of positive control wells (10 μM BI-D1870)
D̅a̅t̅a̅_{Negative Control}: mean value of negative control well ratio (0.5% DMSO)

TABLE 1
In Vitro Inhibitory Activity of Test Compounds on RSK2 Kinase

| Test | IC₅₀ |
|---|---|
| Compound | (nM) |
| Conrol | 13.13 |
| Compound I | |
| Conrol | 1.08 |
| Compound II | |
| 1 | 0.44 |
| 2 | 0.31 |
| 3 | 0.24 |
| 4 | 0.42 |
| 5 | 0.35 |
| 6 | 174.5 |
| 7 | 99.87 |
| 8 | 0.8 |
| 9 | 43.03 |
| 10 | 0.11 |
| 11 | 0.29 |
| 12 | 0.43 |
| 13 | 5.43 |
| 14 | 1.58 |
| 15 | 0.08 |
| 16 | 0.08 |
| 17 | 0.22 |

The experimental results indicate that the compounds of the present invention have a good inhibitory effect on RSK2 kinase.

### Test Example 2: Test of Compound Inhibition on Triple Negative Breast Cancer Cell Proliferation

The compound or vehicle control (DMSO) was diluted in culture medium in a 96 well plate, with the final concentrations of 50, 25, 12.5, 6.25, 3.13, 1.56, 0.78, 0.39, 0.2, and 0.1 μM. 1×10³ MDA-MB-453 cells were inoculated into each well, and the plate was incubated in the incubator for 5 days. After the incubation period, cell survival was quantified using Cell Counting Kit-8 (CCK-8) reagent. In short, the cells were incubated with CCK8 for 2-4 hours, and then the absorbance at 450 nm and 650 nm was measured. CCK-8 is WST-8 (chemical name: 2-(2-methoxy-4-nitrophenyl)-3-(4-nitrophenyl)-5-(2,4-disulfonate benzene)-2H tetrazole monosodium salt), which is reduced to a highly water-soluble yellow formazan dye by dehydrogenases in cells under the action of electron carrier 1-methoxy-5-methylphenazinium sulfate dimethyl ester (1-Methoxy PMS). The number of generated formazan dye substances is directly proportional to the number of live cells, so this characteristic can be used to calculate the survival percentage, as shown in the following equation:

$$cell survival rate ( \% ) = [ ( As - Ac ) / ( Ab - Ac ) ] \times 100 \% ]$$

As: experimental well (the treatment wells containing cells, different concentrations of drug)
Ab: solvent control well (solvent treatment wells containing cells, 0 concentration of drug)
Ac: blank well (well without cells and drugs)

The cell survival rate data and corresponding compound concentrations were input into GraphPad Prism software to fit and calculate IC₅₀ values.

TABLE 2
Proliferation Inhibitory Activity
of Test Compounds on MDA-MB-453

| Test | IC₅₀ |
|---|---|
| Compound | (μM) |
| Control | 4.599 |
| Compound II | |
| 4 | 2.661 |
| 5 | 2.044 |
| 8 | 5.609 |
| 15 | 2.475 |

The experimental results indicate that the compounds of the present invention have good proliferation inhibitory activity on MDA-MB-453.

### Test Example 3: Pharmacokinetic Study

Pharmacokinetic experiments in mice were conducted using male ICR mice weighed 20-25 g, provided by the Experimental Animal Management Department of Shanghai Family Planning Scientific Research Institute. 3 mice were taken and fasted overnight, and then administered orally by gavage (10 mg/kg). Blood samples were collected before administration, at 15 and 30 minutes as well as 1, 2, 4, 8, and 24 hours after administration; in addition, another 3 mice were taken and administered intravenously (1 mg/kg), and blood samples were collected before administration, at 15 and 30 minutes as well as 1, 2, 4, 8, and 24 hours after administration. blood sample was centrifuged at 6800 g, 2-8° C. for 6 minutes, and then the plasma was collected and stored at −80° C. Plasma at each time point were added 10-20 times the amount of methanol or acetonitrile solution containing internal standard, and mixed by vortex for 1 minute, centrifuged at 18000 rpm at 4° C. for 10 minutes, and then the supernatant was taken to analyze by LC-MS/MS directly. The main pharmacokinetic parameters were analyzed using WinNonlin 7.0 software for non compartmental model analysis.

TABLE 3
Pharmacokinetic Test Results of Tested Compounds in Mice

Pharmacokinetic parameters in mice
(Oral gavage administration) (10 mg/kg)

| | Cₘₐₓ | Tₘₐₓ | AUC₀₋ₜ | T_{1/2} |
|---|---|---|---|---|
| Compound | (ng/mL) | (h) | (h*ng/mL) | (h) |
| Control | 3422 | 0.42 | 4606 | 0.82 |
| Compound II | | | | |
| 5 | 17862 | 0.25 | 35139 | 1.57 |
| 15 | 22767 | 0.5 | 99336 | 2.19 |

The results of mouse pharmacokinetic experiments indicate that the compounds of the present invention have high oral exposure, good pharmacokinetic properties, and good druggability.

All references mentioned in the present invention are cited as references in this application, as if each reference is cited separately. In addition, it should be understood that after reading the above teaching content of the present invention, those skilled in the art can make various changes or modifications to the present invention, and these equivalent forms also fall within the scope of the claims attached to this application.

1. A compound represented by the formula (I), a tautomer, a stereoisomer, a hydrate, a solvate, or a pharmaceutically acceptable salt thereof: 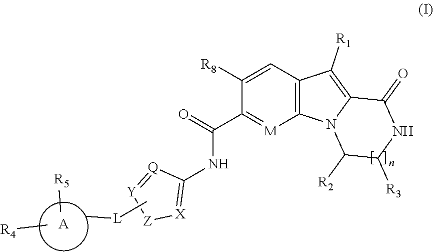
wherein,
n is 1 or 2;
M is independently N or CRₐ;
Rₐ is selected from the group consisting of: halogen, cyano and C₁₋₄ alkoxy;
X, Y and Q are each independently selected from the group consisting of: CR_{b} and N; Z is selected from the group consisting of: S, O and NR_{b}; and 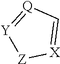
is an aromatic ring, when the connecting site is located at CR_{b} or NR_{b}, the CR_{b} or NR_{b} is C or N;
L is selected from the group consisting of: chemical bond, —C(═O)—, CHR_{b}, C(R_{b})₂, NH and O;
ring A is selected from the group consisting of: C₆₋₁₀ aryl and 5-12 membered heteroaryl;
R₁ is selected from the group consisting of: H, D, halogen, amino and C₁₋₄ alkyl;
R₂ and R₃ are each independently selected from the group consisting of: H, D, C₁₋₆ alkyl, and C₁₋₆ haloalkyl, and R₂ and R₃ is the same or different;
R₄ is selected from the group consisting of: H, D, halogen, hydroxyl, amino, C₂₋₆ sulfonyl, sulfinyl, —N═S(O)(CH₃)₂, 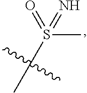 [SMILES: *S(C)(=N)=O]
phosphoryl(-P(O)(CH₃)₂), C₁₋₆ alkyl, C₁₋₆ alkoxy, C₁₋₆ haloalkyl, C₁₋₆ haloalkoxy, C₂₋₆ alkenyl, C₁₋₆ haloalkenyl, C₂₋₆ alkynyl, C₁₋₆ haloalkynyl, C₃₋₆ cycloalkyl, C₆₋₁₀ aryl, 5-12 membered heterocyclyl and 5-12 membered heteroaryl; and the heterocyclyl and heteroaryl independently contain 1-3 heteroatoms selected from N, O, and S;
R₅ is selected from the group consisting of: H, halogen, hydroxyl, amino, sulfonyl, sulfinyl, phosphoryl, C₁₋₆ alkyl, C₁₋₆ alkoxy, C₁₋₆ haloalkyl, C₁₋₆ haloalkoxy, C₂₋₆ alkenyl, C₁₋₆ haloalkenyl, C₂₋₆ alkynyl, C₁₋₆ haloalkynyl, C₃₋₆ cycloalkyl, C₆₋₁₀ aryl, 5-12 membered heterocyclyl, and 5-12 membered heteroaryl; and the heterocyclyl and heteroaryl independently contain 1-3 heteroatoms selected from N, O, and S;
R₈ is selected from the group consisting of: H and halogen;
and, R₁, R₂, R₃, R₄ and R₅ is optionally substituted with one or more R_{b};
R_{b} is selected from the group consisting of: D, halogen, hydroxyl, cyano, amino, imino, C₁₋₄ alkyl, C₁₋₄ alkoxy, C₁₋₄ haloalkyl, C₁₋₄ haloalkoxy, C₂₋₄ alkenyl, C₁₋₄ haloalkenyl, C₂₋₄ alkynyl, C₁₋₄ haloalkynyl, C₃₋₆ cycloalkyl, C₆₋₁₀ aryl and 5-12 membered heteroaryl;
and when the compound has the structure shown in formula I-1 and M is N, ring A is not a phenyl that is unsubstituted or substituted with a substituent selected from the group consisting of: halogenated, C₁₋₆ alkyl, C₁₋₆ haloalkyl-N(R⁷)₂, and —C₁₋₆ alkyl-N(R⁷)₂; wherein, R⁷ is H or C₁₋₆ alkyl;

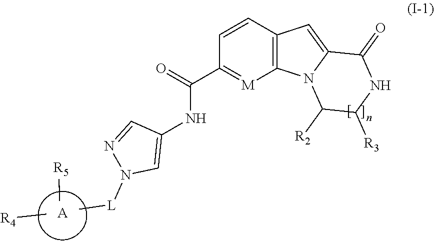

2. The compound of formula I according to claim 1, a tautomer, a stereoisomer, a hydrate, a solvate, or a pharmaceutically acceptable salt thereof, wherein, the compound has the structure shown in formula I-1: 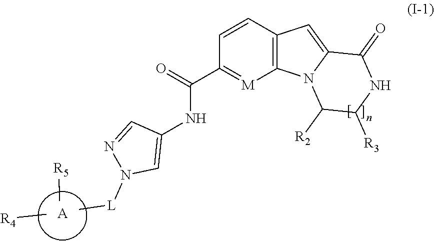
wherein, L is selected from the group consisting of: chemical bond, CH₂, CF₂, CHMe, and C(Me)₂;
when M is N, R₄ is H, D, halogen, C₁₋₆ alkyl, C₁₋₆ alkoxy, C₁₋₆ haloalkyl, or C₁₋₆ haloalkoxy; R₅ is —N═S(O)(CH₃)₂, 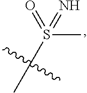 [SMILES: *S(C)(=N)=O]
phosphoryl(-P(O)(CH₃)₂), C₁₋₆ alkyl, or C₃₋₆ cycloalkyl.

3. The compound of formula I according to claim 1, a tautomer, a stereoisomer, a hydrate, a solvate, or a pharmaceutically acceptable salt thereof, wherein, the compound has the structure shown in formula I-2: 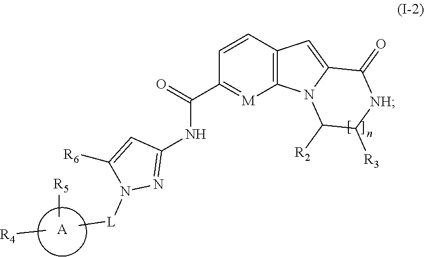
wherein,
L is selected from the group consisting of: chemical bond, CH₂, CF₂, CHMe, and C(Me)₂;
R₆ is selected from the group consisting of: H, D, halogen, C₁₋₆ alkyl, C₁₋₆ alkoxy, C₁₋₆ haloalkyl, C₁₋₆ haloalkoxy, C₂₋₆ alkenyl, C₁₋₆ haloalkenyl, C₂₋₆ alkynyl, C₁₋₆ haloalkynyl, C₃₋₆ cycloalkyl, C₆₋₁₀ aryl, 5-12 membered heterocyclyl, and 5-12 membered heteroaryl; the heterocyclyl and heteroaryl independently contain 1-3 heteroatoms selected from N, O, and S;
and R₆ is optionally substituted with one or more R_{b}.

4. The compound of formula I according to claim 1, a tautomer, a stereoisomer, a hydrate, a solvate, or a pharmaceutically acceptable salt thereof, wherein, the compound has the structure shown in formula I-3: 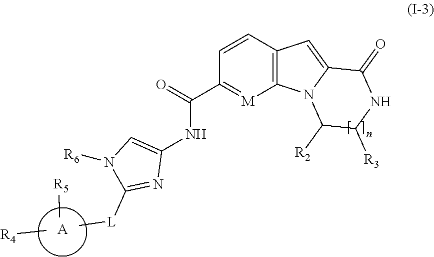
wherein, R₆ is selected from the group consisting of: H, C₁₋₆ alkyl, C₁₋₆ haloalkyl, C₃₋₆ cycloalkyl, C₆₋₁₀ aryl, 5-12 membered heterocyclyl, and 5-12 membered heteroaryl; and the heterocyclyl and heteroaryl independently contain 1-3 heteroatoms selected from N, O, and S; and, R₆ is optionally substituted with one or more R_{b}.

5. The compound of formula I according to claim 1, a tautomer, a stereoisomer, a hydrate, a solvate, or a pharmaceutically acceptable salt thereof, wherein, R₂ and R₃ are selected from the group consisting of: H and methyl, and R₂, R₃ are the same or different.

6. The compound of formula I according to claim 1, a tautomer, a stereoisomer, a hydrate, a solvate, or a pharmaceutically acceptable salt thereof, wherein, R₄ and R₅ are each independently selected from the group consisting of: H, halogen, amino, sulfinyl, phosphoryl, and C₁₋₄ alkyl, and R₄ and R₅ is each independently substituted by one or more R_{b}.

7. A compound selected from the group consisting of: 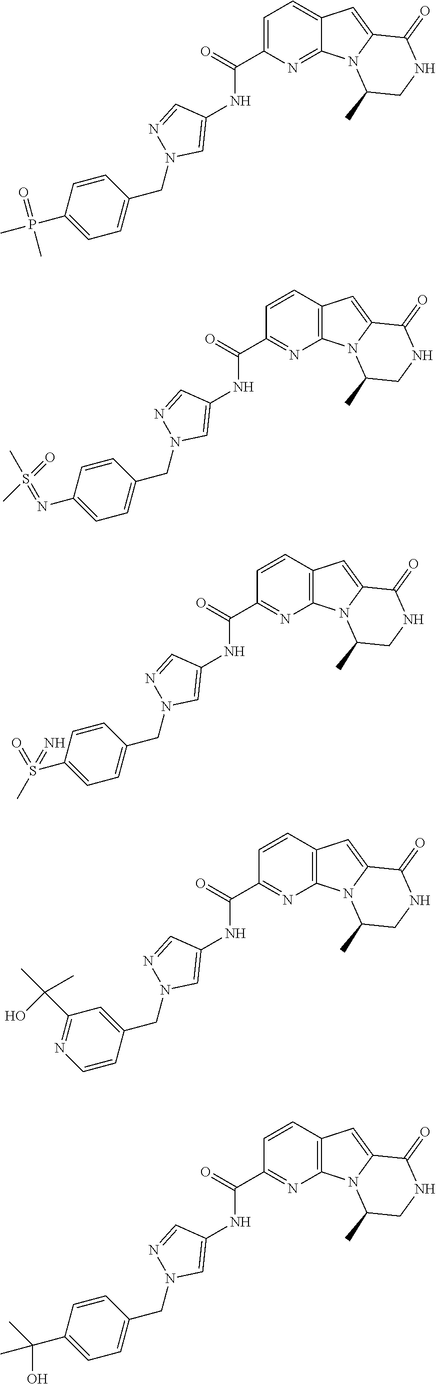 [SMILES: C[C@@H]1CNC(=O)c2cc3ccc(C(=O)Nc4cnn(Cc5ccc(P(C)(C)=O)cc5)c4)nc3n21 ; C[C@@H]1CNC(=O)c2cc3ccc(C(=O)Nc4cnn(Cc5ccc(N=S(C)(C)=O)cc5)c4)nc3n21 ; C[C@@H]1CNC(=O)c2cc3ccc(C(=O)Nc4cnn(Cc5ccc(S(C)(=N)=O)cc5)c4)nc3n21 ; C[C@@H]1CNC(=O)c2cc3ccc(C(=O)Nc4cnn(Cc5ccnc(C(C)(C)O)c5)c4)nc3n21 ; C[C@@H]1CNC(=O)c2cc3ccc(C(=O)Nc4cnn(Cc5ccc(C(C)(C)O)cc5)c4)nc3n21] 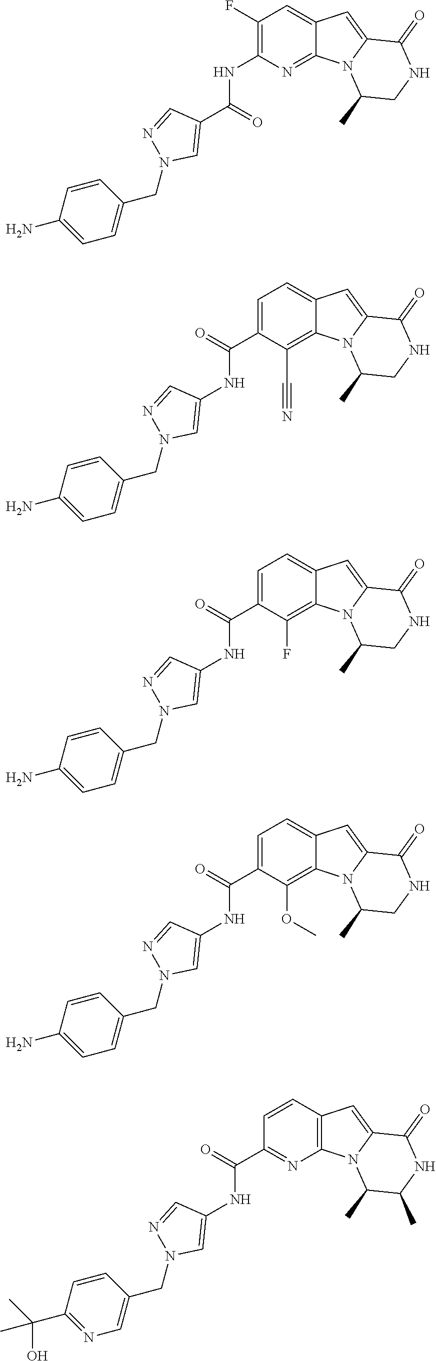 [SMILES: C[C@@H]1CNC(=O)c2cc3cc(F)c(NC(=O)c4cnn(Cc5ccc(N)cc5)c4)nc3n21 ; C[C@@H]1CNC(=O)c2cc3ccc(C(=O)Nc4cnn(Cc5ccc(N)cc5)c4)c(C#N)c3n21 ; C[C@@H]1CNC(=O)c2cc3ccc(C(=O)Nc4cnn(Cc5ccc(N)cc5)c4)c(F)c3n21 ; COc1c(C(=O)Nc2cnn(Cc3ccc(N)cc3)c2)ccc2cc3n(c12)[C@H](C)CNC3=O ; C[C@@H]1NC(=O)c2cc3ccc(C(=O)Nc4cnn(Cc5ccc(C(C)(C)O)nc5)c4)nc3n2[C@@H]1C] 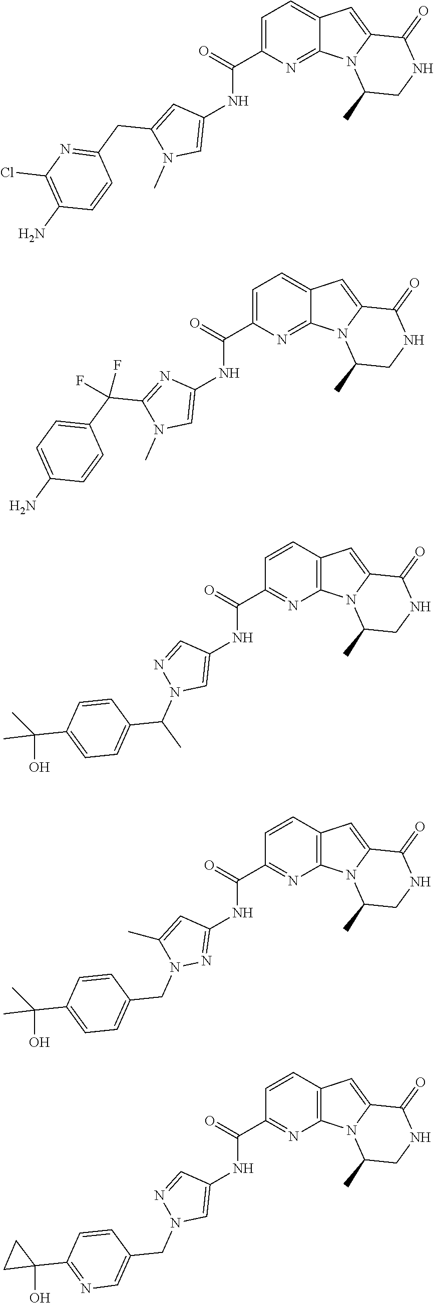 [SMILES: C[C@@H]1CNC(=O)c2cc3ccc(C(=O)Nc4cc(Cc5ccc(N)c(Cl)n5)n(C)c4)nc3n21 ; C[C@@H]1CNC(=O)c2cc3ccc(C(=O)Nc4cn(C)c(C(F)(F)c5ccc(N)cc5)n4)nc3n21 ; CC(c1ccc(C(C)(C)O)cc1)n1cc(NC(=O)c2ccc3cc4n(c3n2)[C@H](C)CNC4=O)cn1 ; Cc1cc(NC(=O)c2ccc3cc4n(c3n2)[C@H](C)CNC4=O)nn1Cc1ccc(C(C)(C)O)cc1 ; C[C@@H]1CNC(=O)c2cc3ccc(C(=O)Nc4cnn(Cc5ccc(C6(O)CC6)nc5)c4)nc3n21] 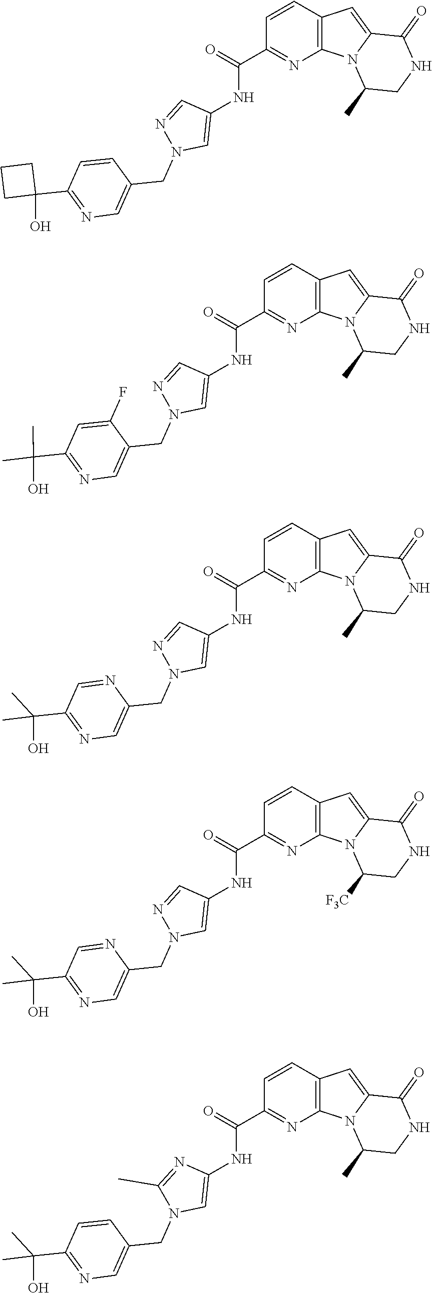 [SMILES: C[C@@H]1CNC(=O)c2cc3ccc(C(=O)Nc4cnn(Cc5ccc(C6(O)CCC6)nc5)c4)nc3n21 ; C[C@@H]1CNC(=O)c2cc3ccc(C(=O)Nc4cnn(Cc5cnc(C(C)(C)O)cc5F)c4)nc3n21 ; C[C@@H]1CNC(=O)c2cc3ccc(C(=O)Nc4cnn(Cc5cnc(C(C)(C)O)cn5)c4)nc3n21 ; CC(C)(O)c1cnc(Cn2cc(NC(=O)c3ccc4cc5n(c4n3)[C@H](C(F)(F)F)CNC5=O)cn2)cn1 ; Cc1nc(NC(=O)c2ccc3cc4n(c3n2)[C@H](C)CNC4=O)cn1Cc1ccc(C(C)(C)O)nc1] 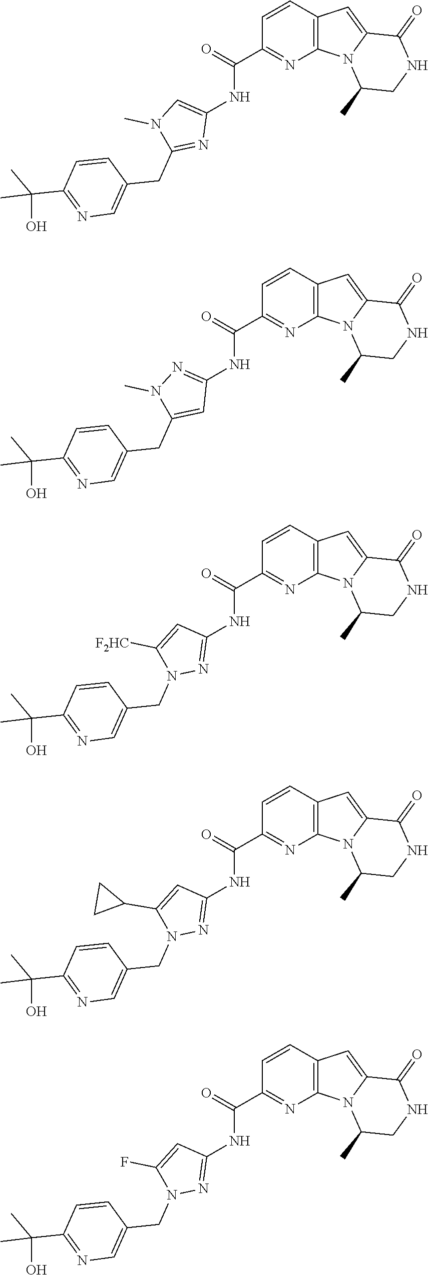 [SMILES: C[C@@H]1CNC(=O)c2cc3ccc(C(=O)Nc4cn(C)c(Cc5ccc(C(C)(C)O)nc5)n4)nc3n21 ; C[C@@H]1CNC(=O)c2cc3ccc(C(=O)Nc4cc(Cc5ccc(C(C)(C)O)nc5)n(C)n4)nc3n21 ; C[C@@H]1CNC(=O)c2cc3ccc(C(=O)Nc4cc(C(F)F)n(Cc5ccc(C(C)(C)O)nc5)n4)nc3n21 ; C[C@@H]1CNC(=O)c2cc3ccc(C(=O)Nc4cc(C5CC5)n(Cc5ccc(C(C)(C)O)nc5)n4)nc3n21 ; C[C@@H]1CNC(=O)c2cc3ccc(C(=O)Nc4cc(F)n(Cc5ccc(C(C)(C)O)nc5)n4)nc3n21] 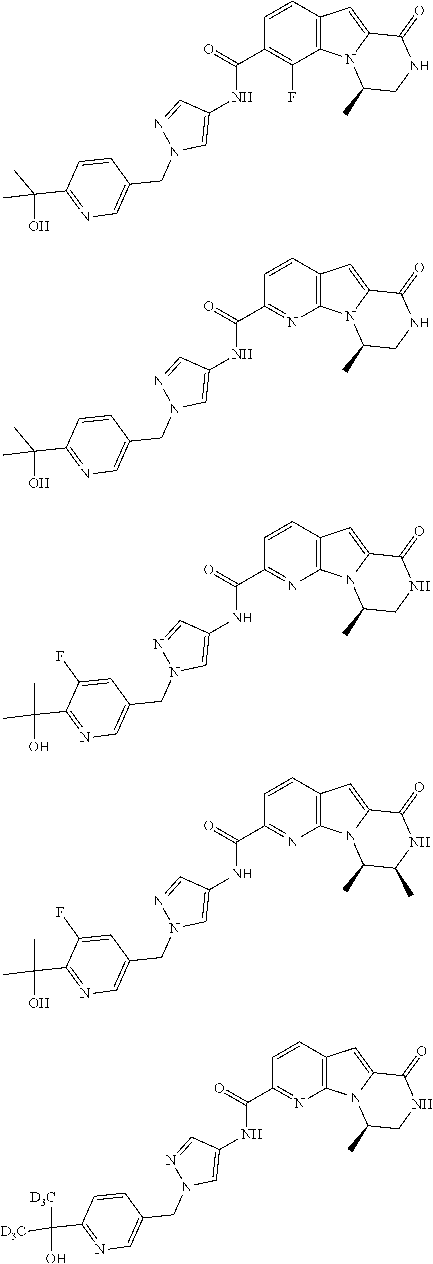 [SMILES: C[C@@H]1CNC(=O)c2cc3ccc(C(=O)Nc4cnn(Cc5ccc(C(C)(C)O)nc5)c4)c(F)c3n21 ; C[C@@H]1CNC(=O)c2cc3ccc(C(=O)Nc4cnn(Cc5ccc(C(C)(C)O)nc5)c4)nc3n21 ; C[C@@H]1CNC(=O)c2cc3ccc(C(=O)Nc4cnn(Cc5cnc(C(C)(C)O)c(F)c5)c4)nc3n21 ; C[C@@H]1NC(=O)c2cc3ccc(C(=O)Nc4cnn(Cc5cnc(C(C)(C)O)c(F)c5)c4)nc3n2[C@@H]1C ; [2H]C([2H])([2H])C(O)(c1ccc(Cn2cc(NC(=O)c3ccc4cc5n(c4n3)[C@H](C)CNC5=O)cn2)cn1)C([2H])([2H])[2H]]
or a tautomer, a stereoisomer, a hydrate, a solvate, or a pharmaceutically acceptable salt thereof.

8. A pharmaceutical composition, wherein, the pharmaceutical composition comprises: one or more of a compound of formula I according to claim 1, a pharmaceutically acceptable salt, a racemate, an R-isomer, an S-isomer, or a mixture thereof, and one or more pharmaceutically acceptable carriers, excipients, adjuvants, excipients, and/or diluents.

9. A method of treating a diseases or condition associated with p90 ribosomal S6 kinase (RSK) activity, the method comprising administering to a subject in need thereof an effective amount of the compound of formula I according to the claim 1, a pharmaceutically acceptable salt, a racemate, an R-isomer, an S-isomer, or a mixture thereof.

10. The method according to claim 9, wherein, the disease or condition is cancer.

11. The method according to claim 10, wherein the cancer is selected from the group consisting of: breast cancer, prostate cancer, lung cancer, brain cancer, skin cancer, bone cancer, ovarian cancer, multiple myeloma and leukemia.
